SEQUENCE LISTING (372 sequences)

SEQ ID NO: 1 - DNA, 354 bp, Homo sapiens
  source 1..354: mol_type = other DNA
caggtgcagctgcaggagtcgggccccggactgatgaagccttcggagaccctgtccctcacctgcactgtctctggtggctccttcagcagttactattggaactggatccggcagtccccagggaagggactggagtggattgggtatatccgttatagtggggacaccaactacaagccctccctcaagagtcgattcaccatatcaattgacacgtccaagaaccttttctccctgaggctgaaatctgtgaccgctgcggacacggccgtgtattactgtgcgaggatgggactggggagtgatgcttttgatatctggggccaagggacaatggtcaccgtctcttca

SEQ ID NO: 2 - protein, 118 aa, Homo sapiens
QVQLQESGPGLMKPSETLSLTCTVSGGSFSSYYWNWIRQSPGKGLEWIGYIRYSGDTNYKPSLKSRFTISIDTSKNLFSLRLKSVTAADTAVYYCARMGLGSDAFDIWGQGTMVTVSS

SEQ ID NO: 3 - DNA, 24 bp, Homo sapiens
  source 1..24: mol_type = other DNA
ggtggctccttcagcagttactat

SEQ ID NO: 4 - protein, 8 aa, Homo sapiens
GGSFSSYY

SEQ ID NO: 5 - DNA, 21 bp, Homo sapiens
  source 1..21: mol_type = other DNA
atccgttatagtggggacacc

SEQ ID NO: 6 - protein, 7 aa, Homo sapiens
IRYSGDT

SEQ ID NO: 7 - DNA, 36 bp, Homo sapiens
  source 1..36: mol_type = other DNA
gcgaggatgggactggggagtgatgcttttgatatc

SEQ ID NO: 8 - protein, 12 aa, Homo sapiens
ARMGLGSDAFDI

SEQ ID NO: 9 - DNA, 324 bp, Homo sapiens
  source 1..324: mol_type = other DNA
gaaattgtgttgacgcagtctccaggcaccctgtctttgtctccaggggaaagagccaccctctcctgcagggccagtcagagtgttaacaacaattatttagcctggtaccagcagaaacctggccaggctcccaggctcctcatctatggtgtattcaacagggccactaacatcccagacaggttcagtggcagtgggtctgggacagatttcactctcaccatcagcagactggagcctgaagattttgcagtgtattactgtcagcagtatggtagttcaccttggacgttcggccaagggaccaaggtggaaatcaaa

SEQ ID NO: 10 - protein, 108 aa, Homo sapiens
EIVLTQSPGTLSLSPGERATLSCRASQSVNNNYLAWYQQKPGQAPRLLIYGVFNRATNIPDRFSGSGSGTDFTLTISRLEPEDFAVYYCQQYGSSPWTFGQGTKVEIK

SEQ ID NO: 11 - DNA, 21 bp, Homo sapiens
  source 1..21: mol_type = other DNA
cagagtgttaacaacaattat

SEQ ID NO: 12 - protein, 7 aa, Homo sapiens
QSVNNNY

SEQ ID NO: 13-14 - skipped (2 entries)

SEQ ID NO: 15 - DNA, 27 bp, Homo sapiens
  source 1..27: mol_type = other DNA
cagcagtatggtagttcaccttggacg

SEQ ID NO: 16 - protein, 9 aa, Homo sapiens
QQYGSSPWT

SEQ ID NO: 17 - DNA, 354 bp, Homo sapiens
  source 1..354: mol_type = other DNA
caggtgcagctgcaggagtcgggcccaagactggtgaagccttcggagaccctgtccctcacctgcattgtctctggtggctccatcagtaatttctactggaactggatccggcagtccccagggaagggactggaatggattggatatttcttttacactgggactatcgactacaacccctccctcaagagtcgagtcaccatatcactggacacgtccaagaaccagttctccctgaacctgcgtcttctgaccgccgcagacgcggccgtttattattgtgcgaggatggggctgggggctaatgcttttgacatctggggccacgggacaatggtcaccgtctcttca

SEQ ID NO: 18 - protein, 118 aa, Homo sapiens
QVQLQESGPRLVKPSETLSLTCIVSGGSISNFYWNWIRQSPGKGLEWIGYFFYTGTIDYNPSLKSRVTISLDTSKNQFSLNLRLLTAADAAVYYCARMGLGANAFDIWGHGTMVTVSS

SEQ ID NO: 19 - DNA, 24 bp, Homo sapiens
  source 1..24: mol_type = other DNA
ggtggctccatcagtaatttctac

SEQ ID NO: 20 - protein, 8 aa, Homo sapiens
GGSISNFY

SEQ ID NO: 21 - DNA, 21 bp, Homo sapiens
  source 1..21: mol_type = other DNA
ttcttttacactgggactatc

SEQ ID NO: 22 - protein, 7 aa, Homo sapiens
FFYTGTI

SEQ ID NO: 23 - DNA, 36 bp, Homo sapiens
  source 1..36: mol_type = other DNA
gcgaggatggggctgggggctaatgcttttgacatc

SEQ ID NO: 24 - protein, 12 aa, Homo sapiens
ARMGLGANAFDI

SEQ ID NO: 25 - DNA, 324 bp, Homo sapiens
  source 1..324: mol_type = other DNA
gaaattgtgttgacgcagtctccaggcaccctgtctttgtctcctggagaaagagccaccctctcctgcagggccagtcagcatgttagcagcaactacttagcctggtaccagcagaaacctggccaggctcccaggctcctcatctatggtggatccagcagggccactggcatcccagacaggttcagtggcagtgggtctgggacagacttcactctcaccatcagcagactggagcctgcagattttgcagtgttttactgtcagcagtatggtaactcaccttggacgttcggccaagggaccaaggtggaaatgaaa

SEQ ID NO: 26 - protein, 108 aa, Homo sapiens
EIVLTQSPGTLSLSPGERATLSCRASQHVSSNYLAWYQQKPGQAPRLLIYGGSSRATGIPDRFSGSGSGTDFTLTISRLEPADFAVFYCQQYGNSPWTFGQGTKVEMK

SEQ ID NO: 27 - DNA, 21 bp, Homo sapiens
  source 1..21: mol_type = other DNA
cagcatgttagcagcaactac

SEQ ID NO: 28 - protein, 7 aa, Homo sapiens
QHVSSNY

SEQ ID NO: 29-30 - skipped (2 entries)

SEQ ID NO: 31 - DNA, 27 bp, Homo sapiens
  source 1..27: mol_type = other DNA
cagcagtatggtaactcaccttggacg

SEQ ID NO: 32 - protein, 9 aa, Homo sapiens
QQYGNSPWT

SEQ ID NO: 33 - DNA, 351 bp, Homo sapiens
  source 1..351: mol_type = other DNA
caggtgcaactacaggagtcgggcccaaaggtggtgaagccttcggagaccctgtccctcacctgcactgtctctggtggctccatcagtagttactactggaattggatccgccagtccccagggaagggactggagtggattggatataccaaaagagggtataccgactacaacccctccctcaggagtcgcgtcactatatcagaagacacgtccaagaaccagttctccctgaggatcagctctgtgaccgccgcagacacggccgtatattactgtgcacaaatggggtggggatcccatgcttttgacatgtggggccaagggacaatggtcgccgtctcttca

SEQ ID NO: 34 - protein, 117 aa, Homo sapiens
QVQLQESGPKVVKPSETLSLTCTVSGGSISSYYWNWIRQSPGKGLEWIGYTKRGYTDYNPSLRSRVTISEDTSKNQFSLRISSVTAADTAVYYCAQMGWGSHAFDMWGQGTMVAVSS

SEQ ID NO: 35 - DNA, 24 bp, Homo sapiens
  source 1..24: mol_type = other DNA
ggtggctccatcagtagttactac

SEQ ID NO: 36 - protein, 8 aa, Homo sapiens
GGSISSYY

SEQ ID NO: 37 - DNA, 18 bp, Homo sapiens
  source 1..18: mol_type = other DNA
accaaaagagggtatacc

SEQ ID NO: 38 - protein, 6 aa, Homo sapiens
TKRGYT

SEQ ID NO: 39 - DNA, 36 bp, Homo sapiens
  source 1..36: mol_type = other DNA
gcacaaatggggtggggatcccatgcttttgacatg

SEQ ID NO: 40 - protein, 12 aa, Homo sapiens
AQMGWGSHAFDM

SEQ ID NO: 41 - DNA, 324 bp, Homo sapiens
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccttggacgttcggccaagggaccaaggtggaaatcaaa

SEQ ID NO: 42 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 43 - DNA, 21 bp, Homo sapiens
  source 1..21: mol_type = other DNA
cagagtgttagcagcagctac

SEQ ID NO: 44 - protein, 7 aa, Homo sapiens
QSVSSSY

SEQ ID NO: 45-46 - skipped (2 entries)

SEQ ID NO: 47 - DNA, 27 bp, Homo sapiens
  source 1..27: mol_type = other DNA
cagcagtatggtagctcaccttggacg

SEQ ID NO: 48 - protein, 9 aa, Homo sapiens
QQYGSSPWT

SEQ ID NO: 49 - DNA, 351 bp, Homo sapiens; identical to SEQ ID NO: 33
  source 1..351: mol_type = other DNA

SEQ ID NO: 50 - protein, 117 aa, Homo sapiens; identical to SEQ ID NO: 34

SEQ ID NO: 51 - DNA, 24 bp, Homo sapiens
  source 1..24: mol_type = other DNA
ggtggctccatcagtagttactac

SEQ ID NO: 52 - protein, 8 aa, Homo sapiens
GGSISSYY

SEQ ID NO: 53 - DNA, 18 bp, Homo sapiens
  source 1..18: mol_type = other DNA
accaaaagagggtatacc

SEQ ID NO: 54 - protein, 6 aa, Homo sapiens
TKRGYT

SEQ ID NO: 55 - DNA, 36 bp, Homo sapiens; identical to SEQ ID NO: 39
  source 1..36: mol_type = other DNA

SEQ ID NO: 56 - protein, 12 aa, Homo sapiens
AQMGWGSHAFDM

SEQ ID NO: 57 - DNA, 324 bp, Homo sapiens
  source 1..324: mol_type = other DNA
gaaattgtgttgacgcagtctccaggcaccctgtctttgtctccaggggaaagagccaccctctcctgcagggccagtcagagtgttaatagtaggtacttagcctggtaccagcagaaacctggccaggctcccaggctcctcatctatggtgcatccagcagggccactggcatcccagacaggtgcagtggcagtgggtccgggacagacttcactctcaccatcagcagactggagcctgaagattttgcagtgtattactgtcagcagtatggtagctcaccttggacgttcggccaggggaccaaggtggaaatcaaa

SEQ ID NO: 58 - protein, 108 aa, Homo sapiens
EIVLTQSPGTLSLSPGERATLSCRASQSVNSRYLAWYQQKPGQAPRLLIYGASSRATGIPDRCSGSGSGTDFTLTISRLEPEDFAVYYCQQYGSSPWTFGQGTKVEIK

SEQ ID NO: 59 - DNA, 21 bp, Homo sapiens
  source 1..21: mol_type = other DNA
cagagtgttaatagtaggtac

SEQ ID NO: 60 - protein, 7 aa, Homo sapiens
QSVNSRY

SEQ ID NO: 61-62 - skipped (2 entries)

SEQ ID NO: 63 - DNA, 27 bp, Homo sapiens
  source 1..27: mol_type = other DNA
cagcagtatggtagctcaccttggacg

SEQ ID NO: 64 - protein, 9 aa, Homo sapiens
QQYGSSPWT

SEQ ID NO: 65 - DNA, 357 bp, Homo sapiens
  source 1..357: mol_type = other DNA
caggtgcagttggtggagtctgggggaggcttggtcaagcctggagggtccctgagactctcctgtgcagcctcgggattcatcttcagtgactactacataaactggattcgccaggctccagggaaggggctggagtgggtttcatatactagcagtagtggtagtaccacttattacgaagactctgtgaagggccgattcaccatgtccagggacaatgccaggaatttagtgtacctgcaaatgaacagcctgagagccgaggacacggccgcgtattactgtgtgagagatctgagatacaacgatggtttggacgtctggggccaagggaccacggtcaccgtctcctca

SEQ ID NO: 66 - protein, 119 aa, Homo sapiens
QVQLVESGGGLVKPGGSLRLSCAASGFIFSDYYINWIRQAPGKGLEWVSYTSSSGSTTYYEDSVKGRFTMSRDNARNLVYLQMNSLRAEDTAAYYCVRDLRYNDGLDVWGQGTTVTVSS

SEQ ID NO: 67 - DNA, 24 bp, Homo sapiens
  source 1..24: mol_type = other DNA
ggattcatcttcagtgactactac

SEQ ID NO: 68 - protein, 8 aa, Homo sapiens
GFIFSDYY

SEQ ID NO: 69 - DNA, 24 bp, Homo sapiens
  source 1..24: mol_type = other DNA
actagcagtagtggtagtaccact

SEQ ID NO: 70 - protein, 8 aa, Homo sapiens
TSSSGSTT

SEQ ID NO: 71 - DNA, 36 bp, Homo sapiens
  source 1..36: mol_type = other DNA
gtgagagatctgagatacaacgatggtttggacgtc

SEQ ID NO: 72 - protein, 12 aa, Homo sapiens
VRDLRYNDGLDV

SEQ ID NO: 73 - DNA, 324 bp, Homo sapiens
  source 1..324: mol_type = other DNA
gacatcctgctgacccagtctccatcctccctgtctgcatctgtaggagacagagtcaccatcacttgccgggcaagtcagagcattcgcacctatttaaattggtatcagcagaaaccagggagagcccctaagctcctggtctatgctgcatccagtttgcaaagtggggtcccatcaaggttcagtggcagtggatctgggacagatttcactctcaccatcagcagtctgcaacctgaagattttgcaacttacttctgtcaacagagttatgataaccctccgatcaccttcggccaggggacacgactggcgattaaa

SEQ ID NO: 74 - protein, 108 aa, Homo sapiens
DILLTQSPSSLSASVGDRVTITCRASQSIRTYLNWYQQKPGRAPKLLVYAASSLQSGVPSRFSGSGSGTDFTLTISSLQPEDFATYFCQQSYDNPPITFGQGTRLAIK

SEQ ID NO: 75 - DNA, 18 bp, Homo sapiens
  source 1..18: mol_type = other DNA
cagagcattcgcacctat

SEQ ID NO: 76 - protein, 6 aa, Homo sapiens
QSIRTY

SEQ ID NO: 77-78 - skipped (2 entries)

SEQ ID NO: 79 - DNA, 30 bp, Homo sapiens
  source 1..30: mol_type = other DNA
caacagagttatgataaccctccgatcacc

SEQ ID NO: 80 - protein, 10 aa, Homo sapiens
QQSYDNPPIT

SEQ ID NO: 81 - DNA, 354 bp, Homo sapiens
  source 1..354: mol_type = other DNA
caggtgcagctgcaggagtcgggcccaggactggtgaagccttcggagaccctgtccctcacctgcactgtctctggtggctccatcagtagttactactggaactggatccggcagtccccagagaagggactggagtggattggatatatctatatcagtgggaccaccaactacaacccctccctcaagagtcgagtcaccatatcagtagacacgttcaagaatcagttctccctgaagctgaactctgtgaccgctgcggacacggccgtgtattactgtgcgaggatggggctggggagggaggcttttgatatctggggccaagggacaatggtcaccgtctcttca

SEQ ID NO: 82 - protein, 118 aa, Homo sapiens
QVQLQESGPGLVKPSETLSLTCTVSGGSISSYYWNWIRQSPEKGLEWIGYIYISGTTNYNPSLKSRVTISVDTFKNQFSLKLNSVTAADTAVYYCARMGLGREAFDIWGQGTMVTVSS

SEQ ID NO: 83 - DNA, 24 bp, Homo sapiens
  source 1..24: mol_type = other DNA
ggtggctccatcagtagttactac

SEQ ID NO: 84 - protein, 8 aa, Homo sapiens
GGSISSYY

SEQ ID NO: 85 - DNA, 21 bp, Homo sapiens
  source 1..21: mol_type = other DNA
atctatatcagtgggaccacc

SEQ ID NO: 86 - protein, 7 aa, Homo sapiens
IYISGTT

SEQ ID NO: 87 - DNA, 36 bp, Homo sapiens
  source 1..36: mol_type = other DNA
gcgaggatggggctggggagggaggcttttgatatc

SEQ ID NO: 88 - protein, 12 aa, Homo sapiens
ARMGLGREAFDI

SEQ ID NO: 89 - DNA, 324 bp, Homo sapiens
  source 1..324: mol_type = other DNA
gaaattgtgttgacgcagtctccaggcaccctgtctttgtctccaggggaaagagccaccctctcctgcagggccagtcagagtgttagcaacaactacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccttggacgttcggccaagggaccaaggtggaaatcaaa

SEQ ID NO: 90 - protein, 108 aa, Homo sapiens
EIVLTQSPGTLSLSPGERATLSCRASQSVSNNYLAWYQQKPGQAPRLLIYGASSRATGIPDRFSGSGSGTDFTLTISRLEPEDFAVYYCQQYGSSPWTFGQGTKVEIK

SEQ ID NO: 91 - DNA, 21 bp, Homo sapiens
  source 1..21: mol_type = other DNA
cagagtgttagcaacaactac

SEQ ID NO: 92 - protein, 7 aa, Homo sapiens
QSVSNNY

SEQ ID NO: 93-94 - skipped (2 entries)

SEQ ID NO: 95 - DNA, 27 bp, Homo sapiens
  source 1..27: mol_type = other DNA
cagcagtatggtagctcaccttggacg

SEQ ID NO: 96 - protein, 9 aa, Homo sapiens
QQYGSSPWT

SEQ ID NO: 97 - DNA, 357 bp, Homo sapiens
  source 1..357: mol_type = other DNA
caggtgcaactggaggagtctgggggaggcgtggtcaaggctggagggtccctgagactctcctgtgtagcctctggattcaccttcagtgactattacataaactggatccgccaggctccagggaaggggctggagtgggtttcgtacattaataatggtggttataccatttactacgcagactctgtgaagggccgattcaccatgtccagggacaacgccaagaactcagtatatctagaaatgaacggcctgagagccgaggacacggccttttattactgtgtgagagacccacggagttattatgggtttgactattggggccagggaaccctggtcaccgtctcctca

SEQ ID NO: 98 - protein, 119 aa, Homo sapiens
QVQLEESGGGVVKAGGSLRLSCVASGFTFSDYYINWIRQAPGKGLEWVSYINNGGYTIYYADSVKGRFTMSRDNAKNSVYLEMNGLRAEDTAFYYCVRDPRSYYGFDYWGQGTLVTVSS

SEQ ID NO: 99 - DNA, 24 bp, Homo sapiens
  source 1..24: mol_type = other DNA
ggattcaccttcagtgactattac

SEQ ID NO: 100 - protein, 8 aa, Homo sapiens
GFTFSDYY

SEQ ID NO: 101 - DNA, 24 bp, Homo sapiens
  source 1..24: mol_type = other DNA
attaataatggtggttataccatt

SEQ ID NO: 102 - protein, 8 aa, Homo sapiens
INNGGYTI

SEQ ID NO: 103 - DNA, 36 bp, Homo sapiens
  source 1..36: mol_type = other DNA
gtgagagacccacggagttattatgggtttgactat

SEQ ID NO: 104 - protein, 12 aa, Homo sapiens
VRDPRSYYGFDY

SEQ ID NO: 105 - DNA, 324 bp, Homo sapiens
  source 1..324: mol_type = other DNA
gacatccagatgacccagtctccatcctccctgtctgcatctgtaggagacagagtcaccatcacttgccgggcaagtcagagcattagcagttatttaaattggaatcagcagaaaccagggaaagccccgaagctcctgatctatgtagcatccagtttgcaaagtggggtcccatcaaggttcagtggcagtggatctgggacagatttcactctcaccatcagcggtctacaacctgaagattttgcaccttactactgtcaacagagttacattacccctccgatcaccttcggccaagggacacgactggatattaaa

SEQ ID NO: 106 - protein, 108 aa, Homo sapiens
DIQMTQSPSSLSASVGDRVTITCRASQSISSYLNWNQQKPGKAPKLLIYVASSLQSGVPSRFSGSGSGTDFTLTISGLQPEDFAPYYCQQSYITPPITFGQGTRLDIK

SEQ ID NO: 107 - DNA, 18 bp, Homo sapiens
  source 1..18: mol_type = other DNA
cagagcattagcagttat

SEQ ID NO: 108 - protein, 6 aa, Homo sapiens
QSISSY

SEQ ID NO: 109-110 - skipped (2 entries)

SEQ ID NO: 111 - DNA, 30 bp, Homo sapiens
  source 1..30: mol_type = other DNA
caacagagttacattacccctccgatcacc

SEQ ID NO: 112 - protein, 10 aa, Homo sapiens
QQSYITPPIT

SEQ ID NO: 113 - DNA, 357 bp, Homo sapiens
  source 1..357: mol_type = other DNA
caggtgcaactggaggagtctgggggaggcgtggtcaaggctggagggtccctgagactctcctgtgcagcctctggattcaccttcagtgactattacataaattggatccgccaggctccagggaaggggctggagtgggtttcgtacattaataatggtggttataccatatactacgcagactctgtgaagggccgattcaccatgtccagggacaacgccaagaactcagtatatctgcaaatgaacggcctgagagccgaggacacggccttttattactgtgtgagagacccacggagttattatgggtttgactattggggccagggaaccctggtcaccgtctcctca

SEQ ID NO: 114 - protein, 119 aa, Homo sapiens
QVQLEESGGGVVKAGGSLRLSCAASGFTFSDYYINWIRQAPGKGLEWVSYINNGGYTIYYADSVKGRFTMSRDNAKNSVYLQMNGLRAEDTAFYYCVRDPRSYYGFDYWGQGTLVTVSS

SEQ ID NO: 115 - DNA, 24 bp, Homo sapiens
  source 1..24: mol_type = other DNA
ggattcaccttcagtgactattac

SEQ ID NO: 116 - protein, 8 aa, Homo sapiens
GFTFSDYY

SEQ ID NO: 117 - DNA, 24 bp, Homo sapiens
  source 1..24: mol_type = other DNA
attaataatggtggttataccata

SEQ ID NO: 118 - protein, 8 aa, Homo sapiens
INNGGYTI

SEQ ID NO: 119 - DNA, 36 bp, Homo sapiens; identical to SEQ ID NO: 103
  source 1..36: mol_type = other DNA

SEQ ID NO: 120 - protein, 12 aa, Homo sapiens
VRDPRSYYGFDY

SEQ ID NO: 121 - DNA, 324 bp, Homo sapiens
  source 1..324: mol_type = other DNA
gacatccagatgacccagtctccatcctccctgtctgcatctgtaggagacagagtcaccatcacttgccgggcaagtcagagtattagcaggtttttacattggtatcagcagaaaccagggaaagcccctaagctcctgatctatggtgcatccagtttgcaaagtggggtcccatcaaggttcagtggcagtggatctgggacagatttcactctcaccatcagcactctgcaacctgaagactttgcaacttactactgtcaacagagttatagtacccctccgatcactttcggccaagggacacgactggagattaaa

SEQ ID NO: 122 - protein, 108 aa, Homo sapiens
DIQMTQSPSSLSASVGDRVTITCRASQSISRFLHWYQQKPGKAPKLLIYGASSLQSGVPSRFSGSGSGTDFTLTISTLQPEDFATYYCQQSYSTPPITFGQGTRLEIK

SEQ ID NO: 123 - DNA, 18 bp, Homo sapiens
  source 1..18: mol_type = other DNA
cagagtattagcaggttt

SEQ ID NO: 124 - protein, 6 aa, Homo sapiens
QSISRF

SEQ ID NO: 125-126 - skipped (2 entries)

SEQ ID NO: 127 - DNA, 30 bp, Homo sapiens
  source 1..30: mol_type = other DNA
caacagagttatagtacccctccgatcact

SEQ ID NO: 128 - protein, 10 aa, Homo sapiens
QQSYSTPPIT

SEQ ID NO: 129 - DNA, 351 bp, Homo sapiens
  source 1..351: mol_type = other DNA
caggtgcacctggtggagtctgggggaggcttggtcaagcctggagggtccctgagactctcctgtgcagcctctggattcatcttcagtgactactacatgagctggatccgacaggctccagggaaggggctggagtgggtttcatacatcagaggtagtggttataacgcagactctgtgcagggtcggttcaccatctccagggacaacgccaagaactcactgtttctgcaaatgaacagcctgagagccgaggacacggccgtgtattactgtgcgagaggatacagctttggttacggcttctttgactactggggccagggaaccctggtcaccgtctcctca

SEQ ID NO: 130 - protein, 117 aa, Homo sapiens
QVHLVESGGGLVKPGGSLRLSCAASGFIFSDYYMSWIRQAPGKGLEWVSYIRGSGYNADSVQGRFTISRDNAKNSLFLQMNSLRAEDTAVYYCARGYSFGYGFFDYWGQGTLVTVSS

SEQ ID NO: 131 - DNA, 24 bp, Homo sapiens
  source 1..24: mol_type = other DNA
ggattcatcttcagtgactactac

SEQ ID NO: 132 - protein, 8 aa, Homo sapiens
GFIFSDYY

SEQ ID NO: 133 - DNA, 18 bp, Homo sapiens
  source 1..18: mol_type = other DNA
atcagaggtagtggttat

SEQ ID NO: 134 - protein, 6 aa, Homo sapiens
IRGSGY

SEQ ID NO: 135 - DNA, 39 bp, Homo sapiens
  source 1..39: mol_type = other DNA
gcgagaggatacagctttggttacggcttctttgactac

SEQ ID NO: 136 - protein, 13 aa, Homo sapiens
ARGYSFGYGFFDY

SEQ ID NO: 137 - DNA, 324 bp, Homo sapiens
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggagagcccctaagctcctgatctatgctgcatccagtttacaaagtggggtcccatcaaggttcagtggcagtggatctgggacagatttcactctcaccatcagcagtctgcaacctgaagattttgcaacttactactgtcaacagagttacagtcaccctccgatcaccttcggccaagggacacgactggagattaaa

SEQ ID NO: 138 - protein, 108 aa, Homo sapiens
DIQMTQSPSSLSASVGDRVTITCRASQSISSYLNWYQQKPGRAPKLLIYAASSLQSGVPSRFSGSGSGTDFTLTISSLQPEDFATYYCQQSYSHPPITFGQGTRLEIK

SEQ ID NO: 139 - DNA, 18 bp, Homo sapiens
  source 1..18: mol_type = other DNA
cagagcattagcagctat

SEQ ID NO: 140 - protein, 6 aa, Homo sapiens
QSISSY

SEQ ID NO: 141-142 - skipped (2 entries)

SEQ ID NO: 143 - DNA, 30 bp, Homo sapiens
  source 1..30: mol_type = other DNA
caacagagttacagtcaccctccgatcacc

SEQ ID NO: 144 - protein, 10 aa, Homo sapiens
QQSYSHPPIT

SEQ ID NO: 145 - DNA, 357 bp, Homo sapiens
  source 1..357: mol_type = other DNA
caggtgcagctggtggagtctgggggaggcttggtcaagcctggagggtccctgagactctcctgtgcagcctctggattcaccttcactgactacttcatgaactggatccgccaggctccagggaaggggctggagtgggtttcatacattagtaatagtggtgctataaaataccaagcagactctgtgaagggccgattcaccatctccagggacaacgccaagaattctttgtatctgcaaatgaacagcctgagagccgacgacacggccgtgtattactgtgtgagagacccacggagctactatgggtttgactattggggccagggaaccctggtcaccgtctcctca

SEQ ID NO: 146 - protein, 119 aa, Homo sapiens
QVQLVESGGGLVKPGGSLRLSCAASGFTFTDYFMNWIRQAPGKGLEWVSYISNSGAIKYQADSVKGRFTISRDNAKNSLYLQMNSLRADDTAVYYCVRDPRSYYGFDYWGQGTLVTVSS

SEQ ID NO: 147 - DNA, 24 bp, Homo sapiens
  source 1..24: mol_type = other DNA
ggattcaccttcactgactacttc

SEQ ID NO: 148 - protein, 8 aa, Homo sapiens
GFTFTDYF

SEQ ID NO: 149 - DNA, 24 bp, Homo sapiens
  source 1..24: mol_type = other DNA
attagtaatagtggtgctataaaa

SEQ ID NO: 150 - protein, 8 aa, Homo sapiens
ISNSGAIK

SEQ ID NO: 151 - DNA, 36 bp, Homo sapiens
  source 1..36: mol_type = other DNA
gtgagagacccacggagctactatgggtttgactat

SEQ ID NO: 152 - protein, 12 aa, Homo sapiens
VRDPRSYYGFDY

SEQ ID NO: 153 - DNA, 324 bp, Homo sapiens
  source 1..324: mol_type = other DNA
gacatccagatgacccagtctccatcctccctgtctgcatctgtaggagacagagtcaccatcacttgccgggcaagtcagagcattaggaattttttaaattggtatcagcagaaaccagggaaagcccctaggctcctgatctatgctgcatccagtttgcaaagtggggtcccatcaaggttcagtggcggtggatctgggacagatttcactctcaccatcagcagtctgcaacctgaagattttgcaacttactactgtcaacagagttacagtaaccctccgatcaccttcggccaagggacacgactggagattaaa

SEQ ID NO: 154 - protein, 108 aa, Homo sapiens
DIQMTQSPSSLSASVGDRVTITCRASQSIRNFLNWYQQKPGKAPRLLIYAASSLQSGVPSRFSGGGSGTDFTLTISSLQPEDFATYYCQQSYSNPPITFGQGTRLEIK

SEQ ID NO: 155 - DNA, 18 bp, Homo sapiens
  source 1..18: mol_type = other DNA
cagagcattaggaatttt

SEQ ID NO: 156 - protein, 6 aa, Homo sapiens
QSIRNF

SEQ ID NO: 157-158 - skipped (2 entries)

SEQ ID NO: 159 - DNA, 30 bp, Homo sapiens
  source 1..30: mol_type = other DNA
caacagagttacagtaaccctccgatcacc

SEQ ID NO: 160 - protein, 10 aa, Homo sapiens
QQSYSNPPIT

SEQ ID NO: 161 - DNA, 357 bp, Homo sapiens
  source 1..357: mol_type = other DNA
caggtgcaactggaggagtctgggggaggcgtggtcaaggctggagggtccctgagactctcctgtgcagcctctggattcaccttcagtgactattacatgaactggatccgccaggctccagggaaggggctggagtgggtttcgtacattaataatggtggttataccatatactacgcagactctgtgaagggccgattcaccatgtccagggacaacgccaagaactcagtgtatctgcaaatgaacggcctgagagccgaggacacggccttttattactgtgtgagagacccacggagctattatgggtttgactactggggccagggaaccctggtcatcgtctcctca

SEQ ID NO: 162 - protein, 119 aa, Homo sapiens
QVQLEESGGGVVKAGGSLRLSCAASGFTFSDYYMNWIRQAPGKGLEWVSYINNGGYTIYYADSVKGRFTMSRDNAKNSVYLQMNGLRAEDTAFYYCVRDPRSYYGFDYWGQGTLVIVSS

SEQ ID NO: 163 - DNA, 24 bp, Homo sapiens
  source 1..24: mol_type = other DNA
ggattcaccttcagtgactattac

SEQ ID NO: 164 - protein, 8 aa, Homo sapiens
GFTFSDYY

SEQ ID NO: 165 - DNA, 24 bp, Homo sapiens
  source 1..24: mol_type = other DNA
attaataatggtggttataccata

SEQ ID NO: 166 - protein, 8 aa, Homo sapiens
INNGGYTI

SEQ ID NO: 167 - DNA, 36 bp, Homo sapiens
  source 1..36: mol_type = other DNA
gtgagagacccacggagctattatgggtttgactac

SEQ ID NO: 168 - protein, 12 aa, Homo sapiens
VRDPRSYYGFDY

SEQ ID NO: 169 - DNA, 324 bp, Homo sapiens
  source 1..324: mol_type = other DNA
gacatccagatgacccagtctccatcctccctgtctgcatctgtaggagacagagtcaccatcacttgccgggcaagtcagagtattagcaggtttttacattggtatcagcagaaaccagggaaagcccctaagctcctgatctatggtgcatccagtttgcaaagtggggtcccatcaaggttcagtggcagtggatctgggacagatttcactctcaccatcagcagtctgcaacctgaagattttgcaacttactactgtcaacagagttatagcacccctccgatcactttcggccaagggacacgactggagattaaa

SEQ ID NO: 170 - protein, 108 aa, Homo sapiens
DIQMTQSPSSLSASVGDRVTITCRASQSISRFLHWYQQKPGKAPKLLIYGASSLQSGVPSRFSGSGSGTDFTLTISSLQPEDFATYYCQQSYSTPPITFGQGTRLEIK

SEQ ID NO: 171 - DNA, 18 bp, Homo sapiens
  source 1..18: mol_type = other DNA
cagagtattagcaggttt

SEQ ID NO: 172 - protein, 6 aa, Homo sapiens
QSISRF

SEQ ID NO: 173-174 - skipped (2 entries)

SEQ ID NO: 175 - DNA, 30 bp, Homo sapiens
  source 1..30: mol_type = other DNA
caacagagttatagcacccctccgatcact

SEQ ID NO: 176 - protein, 10 aa, Homo sapiens
QQSYSTPPIT

SEQ ID NO: 177 - DNA, 357 bp, Homo sapiens
  source 1..357: mol_type = other DNA
caggtgcaactggtggagtctgggggaggcttggtcaagcctggagggtccctgagactctcctgtgcaacctctggattcaccttcggtgactactacatgaactggtttcgccaggctccagggaaggggctggactgggtttcatacattagtagtagtggtagtaccagatactacgcagactctgtgaggggccgattcaccatctccagggacaacgccaagaattcactgtacctgcaaatgaacagtctgagagccgaggacacggccgtctattactgtgtgagagatctccgctactactacggtatggacgtatggggccaagggaccacggtcaccgtctcctca

SEQ ID NO: 178 - protein, 119 aa, Homo sapiens
QVQLVESGGGLVKPGGSLRLSCATSGFTFGDYYMNWFRQAPGKGLDWVSYISSSGSTRYYADSVRGRFTISRDNAKNSLYLQMNSLRAEDTAVYYCVRDLRYYYGMDVWGQGTTVTVSS

SEQ ID NO: 179 - DNA, 24 bp, Homo sapiens
  source 1..24: mol_type = other DNA
ggattcaccttcggtgactactac

SEQ ID NO: 180 - protein, 8 aa, Homo sapiens
GFTFGDYY

SEQ ID NO: 181 - DNA, 24 bp, Homo sapiens
  source 1..24: mol_type = other DNA
attagtagtagtggtagtaccaga

SEQ ID NO: 182 - protein, 8 aa, Homo sapiens
ISSSGSTR

SEQ ID NO: 183 - DNA, 36 bp, Homo sapiens
  source 1..36: mol_type = other DNA
gtgagagatctccgctactactacggtatggacgta

SEQ ID NO: 184 - protein, 12 aa, Homo sapiens
VRDLRYYYGMDV

SEQ ID NO: 185 - DNA, 324 bp, Homo sapiens; identical to SEQ ID NO: 89
  source 1..324: mol_type = other DNA

SEQ ID NO: 186 - protein, 108 aa, Homo sapiens; identical to SEQ ID NO: 90

SEQ ID NO: 187 - DNA, 21 bp, Homo sapiens
  source 1..21: mol_type = other DNA
cagagtgttagcaacaactac

SEQ ID NO: 188 - protein, 7 aa, Homo sapiens
QSVSNNY

SEQ ID NO: 189-190 - skipped (2 entries)

SEQ ID NO: 191 - DNA, 27 bp, Homo sapiens
  source 1..27: mol_type = other DNA
cagcagtatggtagctcaccttggacg

SEQ ID NO: 192 - protein, 9 aa, Homo sapiens
QQYGSSPWT

SEQ ID NO: 193 - DNA, 351 bp, Homo sapiens
  source 1..351: mol_type = other DNA
caggtgcaactgcaggagtcgggcccaggactggtgaagccttcggagaccctgtccctcacctgcactgtctctggtggctccatcagtagttactactggaactggatccggcagtccccagggaagggactggagtggattgggtataccaaaaaaaggagtaccaactacaacccctccctcaggagtcgagtcactatatcagaagacacgtccaagaaccagttctccctgaagatgagctctgtgaccgccgcagacacggccgtatattactgtgcaaaaatggggtggggatcccatgcttttgatatatggggccaagggacaatggtcaccgtctcttca

SEQ ID NO: 194 - protein, 117 aa, Homo sapiens
QVQLQESGPGLVKPSETLSLTCTVSGGSISSYYWNWIRQSPGKGLEWIGYTKKRSTNYNPSLRSRVTISEDTSKNQFSLKMSSVTAADTAVYYCAKMGWGSHAFDIWGQGTMVTVSS

SEQ ID NO: 195 - DNA, 24 bp, Homo sapiens
  source 1..24: mol_type = other DNA
ggtggctccatcagtagttactac

SEQ ID NO: 196 - protein, 8 aa, Homo sapiens
GGSISSYY

SEQ ID NO: 197 - DNA, 18 bp, Homo sapiens
  source 1..18: mol_type = other DNA
accaaaaaaaggagtacc

SEQ ID NO: 198 - protein, 6 aa, Homo sapiens
TKKRST

SEQ ID NO: 199 - DNA, 36 bp, Homo sapiens
  source 1..36: mol_type = other DNA
gcaaaaatggggtggggatcccatgcttttgatata

SEQ ID NO: 200 - protein, 12 aa, Homo sapiens
AKMGWGSHAFDI

SEQ ID NO: 201 - DNA, 324 bp, Homo sapiens; identical to SEQ ID NO: 41
  source 1..324: mol_type = other DNA

SEQ ID NO: 202 - protein, 108 aa, Homo sapiens; identical to SEQ ID NO: 42

SEQ ID NO: 203 - DNA, 21 bp, Homo sapiens
  source 1..21: mol_type = other DNA
cagagtgttagcagcagctac

SEQ ID NO: 204 - protein, 7 aa, Homo sapiens
QSVSSSY

SEQ ID NO: 205-206 - skipped (2 entries)

SEQ ID NO: 207 - DNA, 27 bp, Homo sapiens
  source 1..27: mol_type = other DNA
cagcagtatggtagctcaccttggacg

SEQ ID NO: 208 - protein, 9 aa, Homo sapiens
QQYGSSPWT

SEQ ID NO: 209 - DNA, 354 bp, Homo sapiens
  source 1..354: mol_type = other DNA
caggtgcagctgcaggaggcgggcccaggactggtgaagccttcggagaccctgtccctcacctgcattgtctctggtggctccatcagtggtttctactggaattggatccggcagcccccagggaagggactggaatggattggatatttcctttacagtgggactatcgactacaacccctccctcaagagccgagtcaccatatcagtggacacgtccaaaaaccaattctttctgaagttgagttttgtgaccgccgctgacacggccgtttattactgtgtgaggatgggactgggggctaatgcttttgacatctggggccgagggacaatggtcaccgtctcttca

SEQ ID NO: 210 - protein, 118 aa, Homo sapiens
QVQLQEAGPGLVKPSETLSLTCIVSGGSISGFYWNWIRQPPGKGLEWIGYFLYSGTIDYNPSLKSRVTISVDTSKNQFFLKLSFVTAADTAVYYCVRMGLGANAFDIWGRGTMVTVSS

SEQ ID NO: 211 - DNA, 24 bp, Homo sapiens
  source 1..24: mol_type = other DNA
ggtggctccatcagtggtttctac

SEQ ID NO: 212 - protein, 8 aa, Homo sapiens
GGSISGFY

SEQ ID NO: 213 - DNA, 21 bp, Homo sapiens
  source 1..21: mol_type = other DNA
ttcctttacagtgggactatc

SEQ ID NO: 214 - protein, 7 aa, Homo sapiens
FLYSGTI

SEQ ID NO: 215 - DNA, 36 bp, Homo sapiens
  source 1..36: mol_type = other DNA
gtgaggatgggactgggggctaatgcttttgacatc

SEQ ID NO: 216 - protein, 12 aa, Homo sapiens
VRMGLGANAFDI

SEQ ID NO: 217 - DNA, 324 bp, Homo sapiens
  source 1..324: mol_type = other DNA
gaaattgtgttgacgcagtctccaggcaccctgtctttgtctccaggggaaagagccaccctctcctgcagggccagtcagactgttagtaggagtttcttagcctggtaccagcagaaacctggccaggctcccaggctcctcatctatggtccatccagcagggccactggcatcccagacaggttcagtggcagtgggtctgggacagatttcactctcaccatcagcagactggagcctgaagattttgcggtgtattactgtcaacaatatggtaactcaccttggacgttcggccaagggaccaaggtggaaatcaaa

SEQ ID NO: 218 - protein, 108 aa, Homo sapiens
EIVLTQSPGTLSLSPGERATLSCRASQTVSRSFLAWYQQKPGQAPRLLIYGPSSRATGIPDRFSGSGSGTDFTLTISRLEPEDFAVYYCQQYGNSPWTFGQGTKVEIK

SEQ ID NO: 219 - DNA, 21 bp, Homo sapiens
  source 1..21: mol_type = other DNA
cagactgttagtaggagtttc

SEQ ID NO: 220 - protein, 7 aa, Homo sapiens
QTVSRSF

SEQ ID NO: 221-222 - skipped (2 entries)

SEQ ID NO: 223 - DNA, 27 bp, Homo sapiens
  source 1..27: mol_type = other DNA
caacaatatggtaactcaccttggacg

SEQ ID NO: 224 - protein, 9 aa, Homo sapiens
QQYGNSPWT

SEQ ID NO: 225 - DNA, 357 bp, Homo sapiens
  source 1..357: mol_type = other DNA
caggtccagctggtggagtctgggggaggcttggtcaagcctggagggtccctgagactctcctgtgcagtttctggattcacctacactgactacttcatgaactggatccgacaggctccagggaaggggctggaatgggtttcatatattagtaatagtggtgctatcaaatactacgcagactctgtgaagggccgattcaccatctccagggacaacgccaagaattctttgtatctgcaaatgaacggcctgagagccgacgacacggccgtgtattactgtgtgagagaccctcgaagctactatgggtttgactattggggccagggaaccctggtcaccgtctcctca

SEQ ID NO: 226 - protein, 119 aa, Homo sapiens
QVQLVESGGGLVKPGGSLRLSCAVSGFTYTDYFMNWIRQAPGKGLEWVSYISNSGAIKYYADSVKGRFTISRDNAKNSLYLQMNGLRADDTAVYYCVRDPRSYYGFDYWGQGTLVTVSS

SEQ ID NO: 227 - DNA, 24 bp, Homo sapiens
  source 1..24: mol_type = other DNA
ggattcacctacactgactacttc

SEQ ID NO: 228 - protein, 8 aa, Homo sapiens
GFTYTDYF

SEQ ID NO: 229 - DNA, 24 bp, Homo sapiens
  source 1..24: mol_type = other DNA
attagtaatagtggtgctatcaaa

SEQ ID NO: 230 - protein, 8 aa, Homo sapiens
ISNSGAIK

SEQ ID NO: 231 - DNA, 36 bp, Homo sapiens
  source 1..36: mol_type = other DNA
gtgagagaccctcgaagctactatgggtttgactat

SEQ ID NO: 232 - protein, 12 aa, Homo sapiens
VRDPRSYYGFDY

SEQ ID NO: 233 - DNA, 324 bp, Homo sapiens
  source 1..324: mol_type = other DNA
gacatccagatgacccagtctccatcctccctgtctgcatctgtaggtgacagagtcaccatcacttgccgggcaagtcagagcattagtaactttttaaattggtatcagcagaaaccagggaaagcccctaggctcctgatctatggtgcatccaatttgcaaagtggggtcccatcaaggttcagtggcggtggatctgggacagatttcactctcaccatcagcagtctgcaacctgcagattttgcaacttactactgtcagcagagtttcagtaaccctccggtcaccttcggccaagggacacgactggagattaac

SEQ ID NO: 234 - protein, 108 aa, Homo sapiens
DIQMTQSPSSLSASVGDRVTITCRASQSISNFLNWYQQKPGKAPRLLIYGASNLQSGVPSRFSGGGSGTDFTLTISSLQPADFATYYCQQSFSNPPVTFGQGTRLEIN

SEQ ID NO: 235 - DNA, 18 bp, Homo sapiens
  source 1..18: mol_type = other DNA
cagagcattagtaacttt

SEQ ID NO: 236 - protein, 6 aa, Homo sapiens
QSISNF

SEQ ID NO: 237-238 - skipped (2 entries)

SEQ ID NO: 239 - DNA, 30 bp, Homo sapiens
  source 1..30: mol_type = other DNA
cagcagagtttcagtaaccctccggtcacc

SEQ ID NO: 240 - protein, 10 aa, Homo sapiens
QQSFSNPPVT

SEQ ID NO: 241 - DNA, 357 bp, Homo sapiens
  source 1..357: mol_type = other DNA
caggtgcagttggtggagtctgggggaggcttggtccagcctggagggtccctgagactctcctgtgcagcctctggattcatcttcggtgactactacattaactggattcgccaggctccagggaaggggctgcagtgggtttcatatactagtagtagtggtagtaccacttactacgaagactctgtgaagggccgattcaccatgtccagggacaatgccaagaatttagtatacctgcaaatgaacagcctgagagccgaggacacggccgcgtattattgtgtgagagatctgagatacaacgatggtttggacgtctggggccaagggaccacggtcatcgtctcctca

SEQ ID NO: 242 - protein, 119 aa, Homo sapiens
QVQLVESGGGLVQPGGSLRLSCAASGFIFGDYYINWIRQAPGKGLQWVSYTSSSGSTTYYEDSVKGRFTMSRDNAKNLVYLQMNSLRAEDTAAYYCVRDLRYNDGLDVWGQGTTVIVSS

SEQ ID NO: 243 - DNA, 24 bp, Homo sapiens
  source 1..24: mol_type = other DNA
ggattcatcttcggtgactactac

SEQ ID NO: 244 - protein, 8 aa, Homo sapiens
GFIFGDYY

SEQ ID NO: 245 - DNA, 24 bp, Homo sapiens
  source 1..24: mol_type = other DNA
actagtagtagtggtagtaccact

SEQ ID NO: 246 - protein, 8 aa, Homo sapiens
TSSSGSTT

SEQ ID NO: 247 - DNA, 36 bp, Homo sapiens; identical to SEQ ID NO: 71
  source 1..36: mol_type = other DNA

SEQ ID NO: 248 - protein, 12 aa, Homo sapiens
VRDLRYNDGLDV

SEQ ID NO: 249 - DNA, 324 bp, Homo sapiens
  source 1..324: mol_type = other DNA
gacatcctgctgacccagtctccatcctccctgtctgcatttgtaggagacagagtcaccatcacttgccgggcaagtcagaacattcgcacctatctaaattggtatcagcagaaaccagggagagcccctaagctcctggtctatgctgcatccactttgcaaagtggggtcccatcaaggttcagtggcagtggatctgggacagacttcactctcaccatcagcagtctgcaacctgaagattttgcaagttacttctgtcaacagagttacgataaccctccgatcaccttcggccaagggacacgactggcgattaaa

SEQ ID NO: 250 - protein, 108 aa, Homo sapiens
DILLTQSPSSLSAFVGDRVTITCRASQNIRTYLNWYQQKPGRAPKLLVYAASTLQSGVPSRFSGSGSGTDFTLTISSLQPEDFASYFCQQSYDNPPITFGQGTRLAIK

SEQ ID NO: 251 - DNA, 18 bp, Homo sapiens
  source 1..18: mol_type = other DNA
cagaacattcgcacctat

SEQ ID NO: 252 - protein, 6 aa, Homo sapiens
QNIRTY

SEQ ID NO: 253-254 - skipped (2 entries)

SEQ ID NO: 255 - DNA, 30 bp, Homo sapiens
  source 1..30: mol_type = other DNA
caacagagttacgataaccctccgatcacc

SEQ ID NO: 256 - protein, 10 aa, Homo sapiens
QQSYDNPPIT

SEQ ID NO: 257 - DNA, 360 bp, Homo sapiens
  source 1..360: mol_type = other DNA
gaagtgcaactggtggagtctgggggaggcttggtacagcctggcaggtccctgagactctcctgtacagcctctggattcacctttggtgatcatgccatgcactgggtccgacaagctcccgggaagggcctggagggggtctcaagtattaattggaatagtggtatcataggctatgcggagtctgtgaagggccgattcaccatctccagagacaacgccaagaactccctgtatctgcaaatgaacaatctgagagctgaggacacgggcttatattactgtgcaagggatattggcgtggcaacgcttggtattgactactggggccagggaaccctggtcaccgtctcctca

SEQ ID NO: 258 - protein, 120 aa, Homo sapiens
EVQLVESGGGLVQPGRSLRLSCTASGFTFGDHAMHWVRQAPGKGLEGVSSINWNSGIIGYAESVKGRFTISRDNAKNSLYLQMNNLRAEDTGLYYCARDIGVATLGIDYWGQGTLVTVSS

SEQ ID NO: 259 - DNA, 24 bp, Homo sapiens
  source 1..24: mol_type = other DNA
ggattcacctttggtgatcatgcc

SEQ ID NO: 260 - protein, 8 aa, Homo sapiens
GFTFGDHA

SEQ ID NO: 261 - DNA, 24 bp, Homo sapiens
  source 1..24: mol_type = other DNA
attaattggaatagtggtatcata

SEQ ID NO: 262 - protein, 8 aa, Homo sapiens
INWNSGII

SEQ ID NO: 263 - DNA, 39 bp, Homo sapiens
  source 1..39: mol_type = other DNA
gcaagggatattggcgtggcaacgcttggtattgactac

SEQ ID NO: 264 - protein, 13 aa, Homo sapiens
ARDIGVATLGIDY

SEQ ID NO: 265 - DNA, 324 bp, Homo sapiens
  source 1..324: mol_type = other DNA
gacatccagatgacccagtctccatcctccctgtctgcatcagtaggagacagagtcaccatcacttgccgggcaagtcagagcattagcagctactcaaattggtatcagcagaaaccagggaaagcccctaaactcctgatctatgctgcatccagtttgcaaagtggggtcccatcaaggttcagtggcagtggatctgggacagatttcactctcaccatcagcagtctgcaacctgaagattttgcaacttactactgtcaacagagtcacagtacccctccgatcaccttcggccaagggacacgactggagattaaa

SEQ ID NO: 266 - protein, 108 aa, Homo sapiens
DIQMTQSPSSLSASVGDRVTITCRASQSISSYSNWYQQKPGKAPKLLIYAASSLQSGVPSRFSGSGSGTDFTLTISSLQPEDFATYYCQQSHSTPPITFGQGTRLEIK

SEQ ID NO: 267 - DNA, 18 bp, Homo sapiens
  source 1..18: mol_type = other DNA
cagagcattagcagctac

SEQ ID NO: 268 - protein, 6 aa, Homo sapiens
QSISSY

SEQ ID NO: 269-270 - skipped (2 entries)

SEQ ID NO: 271 - DNA, 30 bp, Homo sapiens
  source 1..30: mol_type = other DNA
caacagagtcacagtacccctccgatcacc

SEQ ID NO: 272 - protein, 10 aa, Homo sapiens
QQSHSTPPIT

SEQ ID NO: 273 - DNA, 357 bp, Homo sapiens
  source 1..357: mol_type = other DNA
caggtgcagctggtggagtctgggggaggcttggtcaagcctggagggtccctgagactctcctgtgcaacctctggattcaccttcactgactacttcatgaattggatccgccaggctccagggaagggactggagtgggtttcatacattagtaacagtggttctatcagatattatgcagactctgtgaggggccgattctccatctccagggacaatgccaagaactccctgtatctgcaaatgagcagcctgagagccgatgacacggccgtgtattactgtgtgagagatccgcgacaaatctacggtatggacgtctggggccaagggaccacggtcaccgtctcctca

SEQ ID NO: 274 - protein, 119 aa, Homo sapiens
QVQLVESGGGLVKPGGSLRLSCATSGFTFTDYFMNWIRQAPGKGLEWVSYISNSGSIRYYADSVRGRFSISRDNAKNSLYLQMSSLRADDTAVYYCVRDPRQIYGMDVWGQGTTVTVSS

SEQ ID NO: 275 - DNA, 24 bp, Homo sapiens
  source 1..24: mol_type = other DNA
ggattcaccttcactgactacttc

SEQ ID NO: 276 - protein, 8 aa, Homo sapiens
GFTFTDYF

SEQ ID NO: 277 - DNA, 24 bp, Homo sapiens
  source 1..24: mol_type = other DNA
attagtaacagtggttctatcaga

SEQ ID NO: 278 - protein, 8 aa, Homo sapiens
ISNSGSIR

SEQ ID NO: 279 - DNA, 36 bp, Homo sapiens
  source 1..36: mol_type = other DNA
gtgagagatccgcgacaaatctacggtatggacgtc

SEQ ID NO: 280 - protein, 12 aa, Homo sapiens
VRDPRQIYGMDV

SEQ ID NO: 281 - DNA, 324 bp, Homo sapiens
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 282 - protein, 108 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 283 - DNA, 18 bp, Homo sapiens
  source 1..18: mol_type = other DNA
cagagcattagcagctat

SEQ ID NO: 284 - protein, 6 aa, Homo sapiens
QSISSY

SEQ ID NO: 285-286 - skipped (2 entries)

SEQ ID NO: 287 - DNA, 30 bp, Homo sapiens
  source 1..30: mol_type = other DNA
caacagagttacagtacccctccgatcacc

SEQ ID NO: 288 - protein, 10 aa, Homo sapiens
QQSYSTPPIT

SEQ ID NO: 289 - DNA, 357 bp, Homo sapiens
  source 1..357: mol_type = other DNA
caggtgcagctggtggagtctgggggaggcttggtcaagcctcgagggtccctgagactctcctgtgcagcctctggattcatcttcgatgactacttcatgaactggatccgccaggctccagggaaggggctggagtgggtttcatacattagtaatagtggtaataccaaatactacgcagactctgtgaagggccgattcaccatctccagggacaacgcctacaactctctgtatctgcaaatgaacagcctgagagccgaggacacggccgtgtattactgtgtgagagatcctagaagttactacggtatggacgtctggggccaagggaccacggtcaccgtctcctca

SEQ ID NO: 290 - protein, 119 aa, Homo sapiens
QVQLVESGGGLVKPRGSLRLSCAASGFIFDDYFMNWIRQAPGKGLEWVSYISNSGNTKYYADSVKGRFTISRDNAYNSLYLQMNSLRAEDTAVYYCVRDPRSYYGMDVWGQGTTVTVSS

SEQ ID NO: 291 - DNA, 24 bp, Homo sapiens
  source 1..24: mol_type = other DNA
ggattcatcttcgatgactacttc

SEQ ID NO: 292 - protein, 8 aa, Homo sapiens
GFIFDDYF

SEQ ID NO: 293 - DNA, 24 bp, Homo sapiens
  source 1..24: mol_type = other DNA
attagtaatagtggtaataccaaa

SEQ ID NO: 294 - protein, 8 aa, Homo sapiens
ISNSGNTK

SEQ ID NO: 295 - DNA, 36 bp, Homo sapiens
  source 1..36: mol_type = other DNA
gtgagagatcctagaagttactacggtatggacgtc

SEQ ID NO: 296 - protein, 12 aa, Homo sapiens
VRDPRSYYGMDV

SEQ ID NO: 297 - DNA, 357 bp, Homo sapiens
  source 1..357: mol_type = other DNA
caggtgcagctggtggagtctgggggaggcttggtcaagcctggagggtccctgagactctcctgtgcagcctctggattcaccttcagtgactactacatgaactggatccgccaggctccagggaaggggctggagtgggtttcatacattaatggtggtggttataccatgtactacgcagactctgtgaagggccgattcaccatctccagggacaacgccaagaactcactttatctgcaaatgaacagcctgagagccgaggacacggccgtgtattactgtgcgagagatagaaggactacagtatctattgactactggggccagggaaccctggtcaccgtctcctca

SEQ ID NO: 298 - protein, 39 aa, Homo sapiens
LQMNSLRAEDTAVYYCARDRRTTVSIDYWGQGTLVTVSS

SEQ ID NO: 299 - DNA, 24 bp, Homo sapiens
  source 1..24: mol_type = other DNA
ggattcaccttcagtgactactac

SEQ ID NO: 300 - protein, 8 aa, Homo sapiens
GFTFSDYY

SEQ ID NO: 301 - DNA, 24 bp, Homo sapiens
  source 1..24: mol_type = other DNA
attaatggtggtggttataccatg

SEQ ID NO: 302 - protein, 8 aa, Homo sapiens
INGGGYTM

SEQ ID NO: 303 - DNA, 36 bp, Homo sapiens
  source 1..36: mol_type = other DNA
gcgagagatagaaggactacagtatctattgactac

SEQ ID NO: 304 - protein, 12 aa, Homo sapiens
ARDRRTTVSIDY

SEQ ID NO: 305 - DNA, 360 bp, Homo sapiens
  source 1..360: mol_type = other DNA
gaagtgcagctggtggagtctgggggagccttggtacagcctggcaggtccctgagactctcctgtgcagcctctggattcacctttgatgattatgccatgcactgggtccggcaagctccagggaagggcctagagtgggtctcaggtattagttggaatagtggtaccatgggctatgcggactctgtgaagggccgattcaccatttccagagacaacgccaagaagtccctgtatctgcaaatgaatagtctgagagctgaggacacggccttgtattactgtggaaaaggtctacttcaccagtgggaggtacttgactactggggccagggaaccctggtcaccgtctcctca

SEQ ID NO: 306 - protein, 120 aa, Homo sapiens
EVQLVESGGALVQPGRSLRLSCAASGFTFDDYAMHWVRQAPGKGLEWVSGISWNSGTMGYADSVKGRFTISRDNAKKSLYLQMNSLRAEDTALYYCGKGLLHQWEVLDYWGQGTLVTVSS

SEQ ID NO: 307 - DNA, 24 bp, Homo sapiens
  source 1..24: mol_type = other DNA
ggattcacctttgatgattatgcc

SEQ ID NO: 308 - protein, 8 aa, Homo sapiens
GFTFDDYA

SEQ ID NO: 309 - DNA, 24 bp, Homo sapiens
  source 1..24: mol_type = other DNA
attagttggaatagtggtaccatg

SEQ ID NO: 310 - protein, 8 aa, Homo sapiens
ISWNSGTM

SEQ ID NO: 311 - DNA, 39 bp, Homo sapiens
  source 1..39: mol_type = other DNA
ggaaaaggtctacttcaccagtgggaggtacttgactac

SEQ ID NO: 312 - protein, 13 aa, Homo sapiens
GKGLLHQWEVLDY

SEQ ID NO: 313 - DNA, 357 bp, Homo sapiens
  source 1..357: mol_type = other DNA
caggtgcagctggtggagtctgggggaggcttggtcaagcctggagggtccctgagactctcctgtgcagcctctggattcatcttcactgattacttcatgaactggatccgccaggctccaggaaaggggctggagtgggtttcgtacattagtaatagtggtaatattttatactatgcagactctgtgaagggccgattcaccatctccagggacaacgccaaggcgtccctgtatctgcaaatgagcagccttaaagccgaggacacggccgtgtattactgtgtgagagatccccgtatgttctacggtatggacgtctggggccaggggaccacggtcaccgtctcctca

SEQ ID NO: 314 - protein, 119 aa, Homo sapiens
QVQLVESGGGLVKPGGSLRLSCAASGFIFTDYFMNWIRQAPGKGLEWVSYISNSGNILYYADSVKGRFTISRDNAKASLYLQMSSLKAEDTAVYYCVRDPRMFYGMDVWGQGTTVTVSS

SEQ ID NO: 315 - DNA, 24 bp, Homo sapiens
  source 1..24: mol_type = other DNA
ggattcatcttcactgattacttc

SEQ ID NO: 316 - protein, 8 aa, Homo sapiens
GFIFTDYF

SEQ ID NO: 317 - DNA, 24 bp, Homo sapiens
  source 1..24: mol_type = other DNA
attagtaatagtggtaatatttta

SEQ ID NO: 318 - protein, 8 aa, Homo sapiens
ISNSGNIL

SEQ ID NO: 319 - DNA, 36 bp, Homo sapiens
  source 1..36: mol_type = other DNA
gtgagagatccccgtatgttctacggtatggacgtc

SEQ ID NO: 320 - protein, 12 aa, Homo sapiens
VRDPRMFYGMDV

SEQ ID NO: 321 - DNA, 357 bp, Homo sapiens
  source 1..357: mol_type = other DNA
caggtgcagctggtggagtctgggggaggcttggtcaagcctggagggtccctgagactctcctgtgcagcctctggattcaccttcagtgactactatatgaattggatccgccaggctccagggaagggactggagtggattacatacattaataatggtggctataccaaatactacgcagactctgtgaagggccgattcatcatctccagggacaacaccaagaattcagtgcatttgcaaatgaatagcctgagagccgaggacacggccgtgtattactgtgtgagagatccccgtatgtattatggtatggacgtctggggccaagggaccacggtcaccgtctcctca

SEQ ID NO: 322 - protein, 119 aa, Homo sapiens
QVQLVESGGGLVKPGGSLRLSCAASGFTFSDYYMNWIRQAPGKGLEWITYINNGGYTKYYADSVKGRFIISRDNTKNSVHLQMNSLRAEDTAVYYCVRDPRMYYGMDVWGQGTTVTVSS

SEQ ID NO: 323 - DNA, 24 bp, Homo sapiens
  source 1..24: mol_type = other DNA
ggattcaccttcagtgactactat

SEQ ID NO: 324 - protein, 8 aa, Homo sapiens
GFTFSDYY

SEQ ID NO: 325 - DNA, 24 bp, Homo sapiens
  source 1..24: mol_type = other DNA
attaataatggtggctataccaaa

SEQ ID NO: 326 - protein, 8 aa, Homo sapiens
INNGGYTK

SEQ ID NO: 327 - DNA, 36 bp, Homo sapiens
  source 1..36: mol_type = other DNA
gtgagagatccccgtatgtattatggtatggacgtc

SEQ ID NO: 328 - protein, 12 aa, Homo sapiens
VRDPRMYYGMDV

SEQ ID NO: 329 - DNA, 357 bp, Homo sapiens
  source 1..357: mol_type = other DNA
caggtgcagctggtggagtctgggggaggcttggtcaagcctggagggtccctgagactctcctgtgcagcctctggattctccttcagtgactactacattacctggatccgccaggctccagggaaggggctggagtgggtttcatacattaatactggtggttataccatgtattacgcagactctgtgaagggccgattcaccatctccagggacaacgccaagaactcactgtatcttcaaatgaacaacctgagagccgaggacacggccgtgtattattgtacgagagataggagatacaactttggtcttgactactggggccagggaaccctggtcaccgtctcctca

SEQ ID NO: 330 - protein, 119 aa, Homo sapiens
QVQLVESGGGLVKPGGSLRLSCAASGFSFSDYYITWIRQAPGKGLEWVSYINTGGYTMYYADSVKGRFTISRDNAKNSLYLQMNNLRAEDTAVYYCTRDRRYNFGLDYWGQGTLVTVSS

SEQ ID NO: 331 - DNA, 24 bp, Homo sapiens
  source 1..24: mol_type = other DNA
ggattctccttcagtgactactac

SEQ ID NO: 332 - protein, 8 aa, Homo sapiens
GFSFSDYY

SEQ ID NO: 333 - DNA, 24 bp, Homo sapiens
  source 1..24: mol_type = other DNA
attaatactggtggttataccatg

SEQ ID NO: 334 - protein, 8 aa, Homo sapiens
INTGGYTM

SEQ ID NO: 335 - DNA, 36 bp, Homo sapiens
  source 1..36: mol_type = other DNA
acgagagataggagatacaactttggtcttgactac

SEQ ID NO: 336 - protein, 12 aa, Homo sapiens
TRDRRYNFGLDY

SEQ ID NO: 337 - protein, 238 aa, Homo sapiens
MAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGAANYTDWEKIPSMSKNRVPDSCCINVTVGCGINFNEKAIHKEGCVEKIGGWLRKNVLVVAAAALGIAFVEVLGIVFACCLVKSIRSGYEVM

SEQ ID NO: 338 - protein, 125 aa, synthetic construct
  REGION 1..125: note = CD63 EC loop 2 MMH
RDKVMSEFNNNFRQQMENYPKNNHTASILDRMQADFKCCGAANYTDWEKIPSMSKNRVPDSCCINVTVGCGINFNEKAIHKEGCVEKIGGWLRKNVLEQKLISEEDLGGEQKLISEEDLHHHHHH

SEQ ID NO: 339 - protein, 324 aa, synthetic construct
  REGION 1..324: note = CD63 EC loop 2 hFC
RDKVMSEFNNNFRQQMENYPKNNHTASILDRMQADFKCCGAANYTDWEKIPSMSKNRVPDSCCINVTVGCGINFNEKAIHKEGCVEKIGGWLRKNVLDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 340 - protein, 330 aa, synthetic construct [sequence omitted]

SEQ ID NO: 341 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 342 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 343 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNRFTQKSLSLSPGK

SEQ ID NO: 344 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 345 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 346 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 347 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 348 - protein, 327 aa, synthetic construct
  REGION 1..327: note = CH sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 349 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 350 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 351 - protein, 327 aa, synthetic construct
  REGION 1..327: note = CH sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 352 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 353 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 354 - protein, 228 aa, synthetic construct
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 355 - protein, 228 aa, synthetic construct
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNRFTQKSLSLSPGK

SEQ ID NO: 356 - protein, 229 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 357 - protein, 229 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 358 - protein, 228 aa, synthetic construct
  REGION 1..228: note = Fc sequence
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 359 - protein, 231 aa, synthetic construct
EPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 360 - protein, 231 aa, synthetic construct
EPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 361 - protein, 228 aa, synthetic construct
  REGION 1..228: note = Fc sequence
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 362 - protein, 229 aa, synthetic construct
  REGION 1..229: note = Fc sequence
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 363 - protein, 229 aa, synthetic construct
  REGION 1..229: note = Fc sequence
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 364 - protein, 1128 aa, synthetic construct
  REGION 1..1128: note = scFv_H4H123450(VH-linker-VL)_linker_GAA
QVQLQESGPKVVKPSETLSLTCTVSGGSISSYYWNWIRQSPGKGLEWIGYTKRGYTDYNPSLRSRVTISEDTSKNQFSLRISSVTAADTAVYYCAQMGWGSHAFDMWGQGTMVAVSSGGSGGGGSGGGGSGGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 365 - DNA, 141 bp, synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 366 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 367 - DNA, 72 bp, synthetic construct
  source 1..72: mol_type = other DNA
gggggaggctgctggtgaatattaaccaaggtcaccccagttatcggaggagcaaacaggggctaagtccac

SEQ ID NO: 368 - DNA, 224 bp, synthetic construct
  source 1..224: mol_type = other DNA
gtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctga

SEQ ID NO: 369 - protein, 952 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 370 - DNA, 21 bp, synthetic construct
  source 1..21: mol_type = other DNA
ggaacccctagtgatggagtt

SEQ ID NO: 371 - DNA, 16 bp, synthetic construct
  source 1..16: mol_type = other DNA
cggcctcagtgagcga

SEQ ID NO: 372 - DNA, 21 bp, synthetic construct
  source 1..21: mol_type = other DNA
cactccctctctgcgcgctcg

(19) United States
(12) Patent Application Publication
(10) Pub. No.: US 2025/0382377 A1
(43) Pub. Date: Dec. 18, 2025

(54) ANTI-CD63 ANTIBODIES, CONJUGATES, AND USES THEREOF

(71) Applicant: Regeneron Pharmaceuticals, Inc., Tarrytown, NY (US)

(72) Inventors: Katherine Cygnar, New York, NY (US); Andrew Baik, Scarsdale, NY (US); Christopher Schoenherr, Piermont, NY (US); Andrew J. Murphy, Croton-on-Hudson, NY (US)

(73) Assignee: Regeneron Pharmaceuticals, Inc., Tarrytown, NY (US)

(21) Appl. No.: 19/050,406

(22) Filed: Feb. 11, 2025

Related U.S. Application Data
(63) Continuation of application No. 17/056,301, filed on Nov. 17, 2020, now Pat. No. 12,252,544.
(60) Provisional application No. 62/673,098, filed on May 17, 2018.
(60) Provisional application No. 62/681,563, filed on Jun. 6, 2018.
(60) Provisional application No. 62/777,592, filed on Dec. 10, 2018.

(51) Int. Cl. C07K 16/28; A61K 39/00; A61K 48/00; C12N 9/34

(52) U.S. Cl. CPC C07K 16/2896; C12N 9/2428; A61K 2039/505; A61K 48/00; C07K 2317/21; C07K 2317/31; C07K 2317/622; C07K 2317/77; C07K 2317/92; C07K 2319/00

(57) ABSTRACT

Antibodies, portions, and fusion proteins thereof to CD63 are provided. Also provided are nucleic acid sequences encoding same. Also provided are compositions comprising and methods of using same, e.g., for treating a patient in need thereof.

### CROSS-REFERENCE TO RELATED APPLICATIONS

This application is a continuation of U.S. patent application Ser. No. 17/056,301, filed Nov. 17, 2020, which is a U.S. National Phase Application filed under 35 U.S.C. § 371 of PCT Application No. PCT/US2019/032922, filed May 17, 2019, which claims priority to U.S. Provisional Application No. 62/777,592, filed Dec. 10, 2018, U.S. Provisional Patent Application No. 62/681,563, filed Jun. 6, 2018, and U.S. Provisional Patent Application No. 62/673,098, filed May 17, 2018, each of which is herein incorporated by reference in its entirety.

### REFERENCE TO A SEQUENCE LISTING SUBMITTED AS AN XML FILE

The Sequence Listing written in file 623240SEQLIST.xml is 330,993 bytes, was created on Feb. 10, 2025, and is hereby incorporated by reference in its entirety.

### FIELD OF THE INVENTION

This application is generally directed to human antibodies and antigen-binding fragments of human antibodies that bind human CD63, and methods of use thereof, e.g., in methods of treating a disorder in a patient in need thereof. The application also relates to antigen-binding molecules comprising at least an antigen-binding fragment of an anti-CD63 antibody, wherein complexation of the antigen-binding molecule to CD63 mediates internalization of the antigen-binding molecule/CD63 complex. The application further relates to conjugates comprising an anti-CD63 antibody (or antigen-binding molecules comprising an antigen-binding fragment of an anti-CD63 antibody) and a therapeutic agent (e.g., a replacement enzyme, cytotoxic drug, etc.), which conjugates may be useful in treating diseases.

### BACKGROUND

The human antigen CD63, also referred to as lysosome-associated glycoprotein 3 (LAMP3), is a member of the tetraspanin superfamily. Its cell expression is ubiquitous and primarily within intracellular endosomal or lysosomal compartments, although some expression is seen on the cell surface. CD63 is an internalizing protein capable of mediating internalization and transport of targets into endosomal/lysosomal compartments when such targets are physically linked to CD63 in some manner, e.g., via an antigen-binding protein conjugated to a payload or a bispecific antigen binding protein that binds the target and CD63.

There is a need in the art for new anti-human CD63 antibodies, including monovalent antigen-binding fragments thereof for use in bispecific binding proteins, capable of binding CD63 and effecting its internalization.

### SUMMARY

The present invention provides antibodies and antigen-binding fragments thereof that bind to human CD63. The antibodies according to this aspect of the invention are useful, inter alia, for specifically directing the internalization and/or lysosomal trafficking of a target protein, an enzyme (e.g., GAA or GLA), a drug conjugate, etc. As such, this aspect of the invention also provides bispecific antibodies, antigen-binding fragments thereof that bind human CD63, antibody-protein fusion constructs (see, e.g., FIG. 1), and antibody drug conjugates.

Exemplary anti-CD63 antibodies of the present invention are listed in Table 1. Table 1sets forth the amino acid and nucleic acid sequence identifiers of the heavy chain variable regions (HCVRs) and light chain variable regions (LCVRs), as well as heavy chain complementarity determining regions (HCDR1, HCDR2 and HCDR3), and light chain complementarity determining regions (LCDR1, LCDR2 and LCDR3) of the exemplary anti-CD63 antibodies.

The present invention provides antibodies, or antigen-binding fragments thereof, comprising an HCVR comprising an amino acid sequence selected from any of the HCVR amino acid sequences listed in Table 1, or a substantially similar sequence thereof having at least 90%, at least 95%, at least 98% or at least 99% sequence identity thereto.

The present invention also provides antibodies, or antigen-binding fragments thereof, comprising an LCVR comprising an amino acid sequence selected from any of the LCVR amino acid sequences listed in Table 1, or a substantially similar sequence thereof having at least 90%, at least 95%, at least 98% or at least 99% sequence identity thereto.

The present invention also provides antibodies, or antigen-binding fragments thereof, comprising an HCVR and an LCVR amino acid sequence pair (HCVR/LCVR) comprising any of the HCVR amino acid sequences listed in Table 1 paired with any of the LCVR amino acid sequences listed in Table 1. According to certain embodiments, the present invention provides antibodies, or antigen-binding fragments thereof, comprising an HCVR/LCVR amino acid sequence pair contained within any of the exemplary anti-CD63 antibodies listed in Table 1. In certain embodiments, the HCVR/LCVR amino acid sequence pair is selected from the group consisting of SEQ ID NOs: 2/10, SEQ ID NOs: 18/26, SEQ ID NOs: 34/42, SEQ ID NOs: 50/58, SEQ ID NOs: 66/74, SEQ ID NOs: 82/90, SEQ ID NOs: 98/106, SEQ ID NOs: 114/122, SEQ ID NOs: 130/138, SEQ ID NOs: 146/154, SEQ ID NOs: 162/170, SEQ ID NOs: 178/186, SEQ ID NOs: 194/202, SEQ ID NOs: 210/218, SEQ ID NOs: 226/234, SEQ ID NOs: 242/250, SEQ ID NOs: 258/266, SEQ ID NOs: 274/282, SEQ ID NOs:290/282, SEQ ID NOs: 298/282, SEQ ID NOs: 306/282, SEQ ID NOs: 314/282, SEQ ID NOs: 322/282, and SEQ ID NOs: 330/282.

The present invention also provides antibodies, or antigen-binding fragments thereof, comprising a heavy chain CDR1 (HCDR1) comprising an amino acid sequence selected from any of the HCDR1 amino acid sequences listed in Table 1 or a substantially similar sequence thereof having at least 90%, at least 95%, at least 98% or at least 99% sequence identity.

The present invention also provides antibodies, or antigen-binding fragments thereof, comprising a heavy chain CDR2 (HCDR2) comprising an amino acid sequence selected from any of the HCDR2 amino acid sequences listed in Table 1 or a substantially similar sequence thereof having at least 90%, at least 95%, at least 98% or at least 99% sequence identity.

The present invention also provides antibodies, or antigen-binding fragments thereof, comprising a heavy chain CDR3 (HCDR3) comprising an amino acid sequence selected from any of the HCDR3 amino acid sequences listed in Table 1 or a substantially similar sequence thereof having at least 90%, at least 95%, at least 98% or at least 99% sequence identity.

The present invention also provides antibodies, or antigen-binding fragments thereof, comprising a light chain CDR1 (LCDR1) comprising an amino acid sequence selected from any of the LCDR1 amino acid sequences listed in Table 1 or a substantially similar sequence thereof having at least 90%, at least 95%, at least 98% or at least 99% sequence identity.

The present invention also provides antibodies, or antigen-binding fragments thereof, comprising a light chain CDR2 (LCDR2) comprising an amino acid sequence selected from any of the LCDR2 amino acid sequences listed in Table 1 or a substantially similar sequence thereof having at least 90%, at least 95%, at least 98% or at least 99% sequence identity.

The present invention also provides antibodies, or antigen-binding fragments thereof, comprising a light chain CDR3 (LCDR3) comprising an amino acid sequence selected from any of the LCDR3 amino acid sequences listed in Table lor a substantially similar sequence thereof having at least 90%, at least 95%, at least 98% or at least 99% sequence identity.

The present invention also provides antibodies, or antigen-binding fragments thereof, comprising an HCDR3 and an LCDR3 amino acid sequence pair (HCDR3/LCDR3) comprising any of the HCDR3 amino acid sequences listed in Table 1 paired with any of the LCDR3 amino acid sequences listed in Table 1. According to certain embodiments, the present invention provides antibodies, or antigen-binding fragments thereof, comprising an HCDR3/LCDR3 amino acid sequence pair contained within any of the exemplary anti-CD63 antibodies listed in Table 1. In certain embodiments, the HCDR3/LCDR3 amino acid sequence pair is selected from the group consisting of SEQ ID NOs: 8/16, SEQ ID NOs: 24/32, SEQ ID NOs: 40/48, SEQ ID NOs: 56/64, SEQ ID NOs: 72/80, SEQ ID NOs:88/96, SEQ ID NOs: 104/112, SEQ ID NOs:120/128, SEQ ID NOs:136/144, SEQ ID NOs; 152/160, SEQ ID NOs:168/176, SEQ ID NOs: 184/192, SEQ ID NOs:200/208; SEQ ID NOs:216/224; SEQ ID NOs:232/240; SEQ ID NOs:248/256; SEQ ID NOs:264/272; SEQ ID NOs:280/288; SEQ ID NOs:296/288; SEQ ID NOs: 304/288; SEQ ID NOs: 312/288; SEQ ID NOs: 320/288; SEQ ID NOs: 328/288; SEQ ID NOs:336/288.

The present invention also provides antibodies, or antigen-binding fragments thereof, comprising a set of six CDRs (i.e., HCDR1-HCDR2-HCDR3-LCDR1-LCDR2-LCDR3) contained within any of the exemplary anti-CD63 antibodies listed in Table 1. In certain embodiments, the HCDR1-HCDR2-HCDR3-LCDR1-LCDR2-LCDR3 amino acid sequences set is selected from the group consisting of SEQ ID NOs:4-6-8-12-14-16, SEQ ID NOs: 20-22-24-26-28-30-32, SEQ ID NOs:36-38-40-44-46-48, SEQ ID NOs:52-54-56-60-62-64; SEQ ID NOs: 68-70-72-76-78-80; SEQ ID NOs: 84-86-88-92-94-96; SEQ ID NOs: 100-102-104-108-110-112; SEQ ID NOs: 116-118-120-124-126-128; SEQ ID NOs:132-134-136-140-142-144; SEQ ID NOs: 148-150-152-156-158-160; SEQ ID NOs:164-166-168-172-174-176; SEQ ID NOs:180-182-184-188-190-192; SEQ ID NOs: 196-198-200-204-206-208; SEQ ID NOs: 212-214-216-220-222-224; SEQ ID NOs: 228-230-232-236-238-240; SEQ ID NOs: 244-246-248-252-254-256; SEQ ID NOs: 260-262-264-268-270-272; SEQ ID NOs: 276-278-280-284-286-288; SEQ ID NOs: 292-294-296-284-286-288; SEQ ID NOs:300-302-304-284-286-288; SEQ ID NOs: 308-310-312-284-286-288; SEQ ID NOs: 316-318-320-284-286-288; SEQ ID NOs:324-326-328-284-286-288, and SEQ ID NOs:332-334-336-284-286-288.

In a related embodiment, the present invention provides antibodies, or antigen-binding fragments thereof, comprising a set of six CDRs (i.e., HCDR1-HCDR2-HCDR3-LCDR1-LCDR2-LCDR3) contained within an HCVR/LCVR amino acid sequence pair as defined by any of the exemplary anti-CD63 antibodies listed in Table 1. For example, the present invention includes antibodies, or antigen-binding fragments thereof, comprising the HCDR1-HCDR2-HCDR3-LCDR1-LCDR2-LCDR3 amino acid sequences set contained within an HCVR/LCVR amino acid sequence pair selected from the group consisting of SEQ ID NOs: 2/10, SEQ ID NOs: 18/26, SEQ ID NOs: 34/42, SEQ ID NOs: 50/58, SEQ ID NOs: 66/74, SEQ ID NOs: 82/90, SEQ ID NOs: 98/106, SEQ ID NOs: 114/122, SEQ ID NOs: 130/138, SEQ ID NOs: 146/154, SEQ ID NOs: 162/170, SEQ ID NOs: 178/186, SEQ ID NOs: 194/202, SEQ ID NOs: 210/218, SEQ ID NOs: 226/234, SEQ ID NOs: 242/250, SEQ ID NOs: 258/266, SEQ ID NOs: 274/282, SEQ ID NOs:290/282, SEQ ID NOs: 298/282, SEQ ID NOs: 306/282, SEQ ID NOs: 314/282, SEQ ID NOs: 322/282, and SEQ ID NOs: 330/282. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

The present invention also provides nucleic acid molecules encoding anti-CD63 antibodies or portions thereof. For example, the present invention provides nucleic acid molecules encoding any of the HCVR amino acid sequences listed in Table 1; in certain embodiments the nucleic acid molecule comprises a polynucleotide sequence selected from any of the HCVR nucleic acid sequences listed in Table 1, or a substantially similar sequence thereof having at least 90%, at least 95%, at least 98% or at least 99% sequence identity thereto.

The present invention also provides nucleic acid molecules encoding any of the LCVR amino acid sequences listed in Table 1; in certain embodiments the nucleic acid molecule comprises a polynucleotide sequence selected from any of the LCVR nucleic acid sequences listed in Table 1, or a substantially similar sequence thereof having at least 90%, at least 95%, at least 98% or at least 99% sequence identity thereto.

The present invention also provides nucleic acid molecules encoding any of the HCDR1 amino acid sequences listed in Table 1; in certain embodiments the nucleic acid molecule comprises a polynucleotide sequence selected from any of the HCDR1 nucleic acid sequences listed in Table 1, or a substantially similar sequence thereof having at least 90%, at least 95%, at least 98% or at least 99% sequence identity thereto.

The present invention also provides nucleic acid molecules encoding any of the HCDR2 amino acid sequences listed in Table 1; in certain embodiments the nucleic acid molecule comprises a polynucleotide sequence selected from any of the HCDR2 nucleic acid sequences listed in Table 1, or a substantially similar sequence thereof having at least 90%, at least 95%, at least 98% or at least 99% sequence identity thereto.

The present invention also provides nucleic acid molecules encoding any of the HCDR3 amino acid sequences listed in Table 1; in certain embodiments the nucleic acid molecule comprises a polynucleotide sequence selected from any of the HCDR3 nucleic acid sequences listed in Table 1, or a substantially similar sequence thereof having at least 90%, at least 95%, at least 98% or at least 99% sequence identity thereto.

The present invention also provides nucleic acid molecules encoding any of the LCDR1 amino acid sequences listed in Table 1; in certain embodiments the nucleic acid molecule comprises a polynucleotide sequence selected from any of the LCDR1 nucleic acid sequences listed in Table 1, or a substantially similar sequence thereof having at least 90%, at least 95%, at least 98% or at least 99% sequence identity thereto.

The present invention also provides nucleic acid molecules encoding any of the LCDR2 amino acid sequences listed in Table 1; in certain embodiments the nucleic acid molecule comprises a polynucleotide sequence selected from any of the LCDR2 nucleic acid sequences listed in Table 1, or a substantially similar sequence thereof having at least 90%, at least 95%, at least 98% or at least 99% sequence identity thereto.

The present invention also provides nucleic acid molecules encoding any of the LCDR3 amino acid sequences listed in Table 1; in certain embodiments the nucleic acid molecule comprises a polynucleotide sequence selected from any of the LCDR3 nucleic acid sequences listed in Table 1, or a substantially similar sequence thereof having at least 90%, at least 95%, at least 98% or at least 99% sequence identity thereto.

The present invention also provides nucleic acid molecules encoding an HCVR, wherein the HCVR comprises a set of three CDRs (i.e., HCDR1-HCDR2-HCDR3), wherein the HCDR1-HCDR2-HCDR3 amino acid sequence set is as defined by any of the exemplary anti-CD63 antibodies listed in Table 1.

The present invention also provides nucleic acid molecules encoding an LCVR, wherein the LCVR comprises a set of three CDRs (i.e., LCDR1-LCDR2-LCDR3), wherein the LCDR1-LCDR2-LCDR3 amino acid sequence set is as defined by any of the exemplary anti-CD63 antibodies listed in Table 1.

The present invention also provides nucleic acid molecules encoding both an HCVR and an LCVR, wherein the HCVR comprises an amino acid sequence of any of the HCVR amino acid sequences listed in Table 1, and wherein the LCVR comprises an amino acid sequence of any of the LCVR amino acid sequences listed in Table 1. In certain embodiments, the nucleic acid molecule comprises a polynucleotide sequence selected from any of the HCVR nucleic acid sequences listed in Table 1, or a substantially similar sequence thereof having at least 90%, at least 95%, at least 98% or at least 99% sequence identity thereto, and a polynucleotide sequence selected from any of the LCVR nucleic acid sequences listed in Table 1, or a substantially similar sequence thereof having at least 90%, at least 95%, at least 98% or at least 99% sequence identity thereto. In certain embodiments according to this aspect of the invention, the nucleic acid molecule encodes an HCVR and LCVR, wherein the HCVR and LCVR are both derived from the same anti-CD63 antibody listed in Table 1.

The present invention also provides recombinant expression vectors capable of expressing a polypeptide comprising a heavy or light chain variable region of an anti-CD63 antibody. For example, the present invention includes recombinant expression vectors comprising any of the nucleic acid molecules mentioned above, i.e., nucleic acid molecules encoding any of the HCVR, LCVR, and/or CDR sequences as set forth in Table 1. Also included within the scope of the present invention are host cells into which such vectors have been introduced, as well as methods of producing the antibodies or portions thereof by culturing the host cells under conditions permitting production of the antibodies or antibody fragments, and recovering the antibodies and antibody fragments so produced.

In some aspects, the present invention includes antibodies or antigen-binding fragments thereof, such as anti-CD63 antibodies, having a modified glycosylation pattern. In some embodiments, modification to remove undesirable glycosylation sites may be useful, or an antibody lacking a fucose moiety present on the oligosaccharide chain, for example, to increase antibody dependent cellular cytotoxicity (ADCC) function (see Shields et al. (2002) JBC 277:26733), where cytotoxicity is desirable. In other applications, modification of galactosylation can be made in order to modify complement dependent cytotoxicity (CDC).

In another aspect, the invention provides a pharmaceutical composition comprising a recombinant human antibody or fragment thereof which specifically binds CD63 and a pharmaceutically acceptable carrier. In a related aspect, the invention features a composition which is a combination of an anti-CD63 antibody and a second therapeutic agent. In one embodiment, the second therapeutic agent is any agent that is advantageously combined with an anti-CD63 antibody. Additional combination therapies and co-formulations involving the anti-CD63 antibodies of the present invention are disclosed elsewhere herein.

In another aspect, the invention provides therapeutic methods for targeting/killing tumor cells expressing CD63 using an anti-CD63 antibody of the invention, wherein the therapeutic methods comprise administering a therapeutically effective amount of a pharmaceutical composition comprising an anti-CD63 antibody of the invention to a subject in need thereof. In some cases, the anti-CD63 antibodies (or antigen-binding fragments thereof) may be modified to be more cytotoxic by methods, including but not limited to, modified Fc domains to increase ADCC (see e.g. Shields, R. L. et al. (2002) JBC 277:26733), radioimmunotherapy, antibody-drug conjugates, or other methods for increasing the efficiency of tumor ablation.

The present invention also includes the use of an anti-CD63 antibody of the invention in the manufacture of a medicament for the treatment of a disease or disorder (e.g., cancer) related to or caused by CD63-expressing cells. In one aspect, the invention relates to a compound comprising an anti-CD63 antibody or antigen-binding fragment, or a bispecific anti-TAAxCD63 antibody, for use in medicine. In one aspect, the invention relates to a compound comprising an antibody-drug conjugate (ADC) as disclosed herein, for use in medicine.

In yet another aspect, the invention provides monospecific anti-CD63 antibodies for diagnostic applications, such as, e.g., imaging reagents.

In yet another aspect, the invention provides therapeutic methods for enhancing CD63 internalization into and/or degradation by a lysosome using an anti-CD63 antibody or antigen-binding portion of an antibody of the invention, wherein the therapeutic methods comprise administering a therapeutically effective amount of a pharmaceutical composition comprising an antibody.

In another aspect, the present invention provides an antibody or antigen-binding fragment thereof that binds CD63-expressing cells with a KD of greater than 100 nM as measured by surface plasmon resonance, or equivalent assay. In another aspect, the present invention provides an antibody or antigen-binding fragment thereof that binds CD63-expressing cells with an EC50 of greater than 100 nM as measured by FACS analysis. In another aspect, the present invention provides an antibody or antigen-binding fragment thereof that binds and is internalized into lysosomes of CD63-expressing cells in the event that the antibody also binds to CD63 expressed on the same cell.

The invention further provides an antibody or antigen-binding fragment that competes for binding to human CD63 with a reference antibody comprising an HCVR/LCVR amino acid sequence pair as set forth in Table 1. In another aspect, the invention provides an antibody or antigen-binding fragment that competes for binding to human CD63 with a reference antibody comprising an HCVR/LCVR amino acid sequence pair selected from the group consisting of SEQ ID NOs: 2/10, SEQ ID NOs: 18/26, SEQ ID NOs: 34/42, SEQ ID NOs: 50/58, SEQ ID NOs: 66/74, SEQ ID NOs: 82/90, SEQ ID NOs: 98/106, SEQ ID NOs: 114/122, SEQ ID NOs: 130/138, SEQ ID NOs: 146/154, SEQ ID NOs: 162/170, SEQ ID NOs: 178/186, SEQ ID NOs: 194/202, SEQ ID NOs: 210/218, SEQ ID NOs: 226/234, SEQ ID NOs: 242/250, SEQ ID NOs: 258/266, SEQ ID NOs: 274/282, SEQ ID NOs:290/282, SEQ ID NOs: 298/282, SEQ ID NOs: 306/282, SEQ ID NOs: 314/282, SEQ ID NOs: 322/282, and SEQ ID NOs: 330/282.

The invention furthermore provides an antibody or antigen-binding fragment, wherein the antibody or antigen-binding fragment thereof binds to the same epitope on human CD63 as a reference antibody comprising an HCVR/LCVR amino acid sequence pair as set forth in Table 2. In another aspect, the antibody or antigen-binding fragment binds to the same epitope on human CD63 as a reference antibody comprising an HCVR/LCVR amino acid sequence pair selected from the group consisting of SEQ ID NOs: 2/10, SEQ ID NOs: 18/26, SEQ ID NOs: 34/42, SEQ ID NOs: 50/58, SEQ ID NOs: 66/74, SEQ ID NOs: 82/90, SEQ ID NOs: 98/106, SEQ ID NOs: 114/122, SEQ ID NOs: 130/138, SEQ ID NOs: 146/154, SEQ ID NOs: 162/170, SEQ ID NOs: 178/186, SEQ ID NOs: 194/202, SEQ ID NOs: 210/218, SEQ ID NOs: 226/234, SEQ ID NOs: 242/250, SEQ ID NOs: 258/266, SEQ ID NOs: 274/282, SEQ ID NOs:290/282, SEQ ID NOs: 298/282, SEQ ID NOs: 306/282, SEQ ID NOs: 314/282, SEQ ID NOs: 322/282, and SEQ ID NOs: 330/282.

The invention further provides an isolated antibody or antigen-binding fragment thereof that binds human CD63, wherein the antibody or antigen-binding fragment comprises: the complementarity determining regions (CDRs) of a heavy chain variable region (HCVR) having an amino acid sequence as set forth in Table 1; and the CDRs of a light chain variable region (LCVR) having an amino acid sequence as set forth in Table 1. In another aspect, the isolated antibody or antigen-binding fragment comprises the heavy and light chain CDRs of a HCVR/LCVR amino acid sequence pair selected from the group consisting of SEQ ID NOs: 2/10, SEQ ID NOs: 18/26, SEQ ID NOs: 34/42, SEQ ID NOs: 50/58, SEQ ID NOs: 66/74, SEQ ID NOs: 82/90, SEQ ID NOs: 98/106, SEQ ID NOs: 114/122, SEQ ID NOs: 130/138, SEQ ID NOs: 146/154, SEQ ID NOs: 162/170, SEQ ID NOs: 178/186, SEQ ID NOs: 194/202, SEQ ID NOs: 210/218, SEQ ID NOs: 226/234, SEQ ID NOs: 242/250, SEQ ID NOs: 258/266, SEQ ID NOs: 274/282, SEQ ID NOs:290/282, SEQ ID NOs: 298/282, SEQ ID NOs: 306/282, SEQ ID NOs: 314/282, SEQ ID NOs: 322/282, and SEQ ID NOs: 330/282. In yet another aspect, the isolated antibody or antigen-binding fragment comprises HCDR1-HCDR2-HCDR3-LCDR1-LCDR2-LCDR3 domains, respectively, selected from the group consisting SEQ ID NOs: 4-6-8-12-14-16, SEQ ID NOs: 20-22-24-26-28-30-32, SEQ ID NOs:36-38-40-44-46-48, SEQ ID NOs:52-54-56-60-62-64; SEQ ID NOs: 68-70-72-76-78-80; SEQ ID NOs: 84-86-88-92-94-96; SEQ ID NOs: 100-102-104-108-110-112; SEQ ID NOs: 116-118-120-124-126-128; SEQ ID NOs:132-134-136-140-142-144; SEQ ID NOs: 148-150-152-156-158-160; SEQ ID NOs:164-166-168-172-174-176; SEQ ID NOs:180-182-184-188-190-192; SEQ ID NOs: 196-198-200-204-206-208; SEQ ID NOs: 212-214-216-220-222-224; SEQ ID NOs: 228-230-232-236-238-240; SEQ ID NOs: 244-246-248-252-254-256; SEQ ID NOs: 260-262-264-268-270-272; SEQ ID NOs: 276-278-280-284-286-288; SEQ ID NOs: 292-294-296-284-286-288; SEQ ID NOs:300-302-304-284-286-288; SEQ ID NOs: 308-310-312-284-286-288; SEQ ID NOs: 316-318-320-284-286-288; SEQ ID NOs:324-326-328-284-286-288, and SEQ ID NOs:332-334-336-284-286-288. In another aspect, the invention provides an isolated antibody or antigen-binding fragment thereof that binds human CD63, wherein the antibody or antigen-binding fragment comprises: (a) a heavy chain variable region (HCVR) having an amino acid sequence selected from the group consisting of SEQ ID NOs: 2, 18, 34, 50, 66, 82, 98, 114, 130, 146, 162, 178, 194, 210, 226, 242, 258, 274, 290, 298, 306, 314, 322, and 330; and (b) a light chain variable region (LCVR) having an amino acid sequence selected from the group consisting of SEQ ID NOs: 10, 26, 42, 58, 74, 90, 106, 122, 138, 154, 170, 186, 202, 218, 234, 250, 266, and 282. In a further aspect, the isolated antibody or antigen-binding fragment comprises a HCVR/LCVR amino acid sequence pair selected from the group consisting of SEQ ID NOs: SEQ ID NOs: 2/10, SEQ ID NOs: 18/26, SEQ ID NOs: 34/42, SEQ ID NOs: 50/58, SEQ ID NOs: 66/74, SEQ ID NOs: 82/90, SEQ ID NOs: 98/106, SEQ ID NOs: 114/122, SEQ ID NOs: 130/138, SEQ ID NOs: 146/154, SEQ ID NOs: 162/170, SEQ ID NOs: 178/186, SEQ ID NOs: 194/202, SEQ ID NOs: 210/218, SEQ ID NOs: 226/234, SEQ ID NOs: 242/250, SEQ ID NOs: 258/266, SEQ ID NOs: 274/282, SEQ ID NOs:290/282, SEQ ID NOs: 298/282, SEQ ID NOs: 306/282, SEQ ID NOs: 314/282, SEQ ID NOs: 322/282, and SEQ ID NOs: 330/282.

According to another aspect, the present invention provides antibody-drug conjugates comprising an anti-CD63 antibody, antigen-binding fragment thereof, or a multispecific binding protein comprising said antigen-binding fragment thereof, as described herein and a therapeutic agent (e.g., a cytotoxic agent). In some embodiments, the (a) anti-CD63 antibody, antigen-binding fragment and/or multispecific binding protein and (b) the cytotoxic agent are covalently attached via a linker, as discussed herein. In various embodiments, the anti-CD63 antibody or antigen-binding fragment can be any of the anti-CD63 antibodies or fragments described herein.

In some embodiments, the cytotoxic agent is selected from an auristatin, a maytansinoid, a tubulysin, a tomaymycin, calicheamicin, or a dolastatin derivative. In some cases, the cytotoxic agent is an auristatin selected from MMAE or MMAF, or a maytansinoid selected from DM1 or DM4. In some embodiments, the cytotoxic agent is a maytansinoid having the structure of Formulae, as discussed herein.

In some embodiments, the cytotoxic agent is a maytansinoid having the structure:

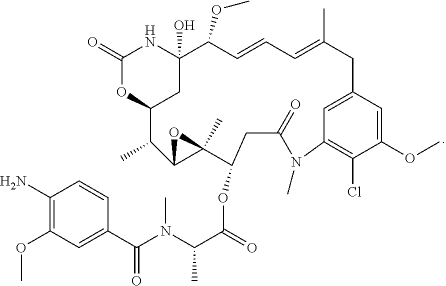 [SMILES: COc1cc(C(=O)N(C)[C@@H](C)C(=O)O[C@H]2CC(=O)N(C)c3cc(cc(OC)c3Cl)C/C(C)=C/C=C/[C@@H](OC)[C@@]3(O)C[C@H](OC(=O)N3)[C@@H](C)[C@@H]3O[C@@]23C)ccc1N]

In some embodiments, the cytotoxic agent is a maytansinoid having the structure:

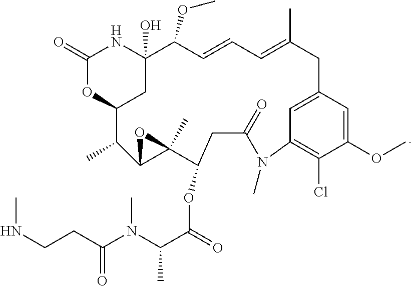 [SMILES: CNCCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

In some embodiments, the antibody-drug conjugate comprises an antigen binding protein, including a multispecific binding protein, comprising an anti-CD63 antigen-binding protein or antigen binding portion thereof, and

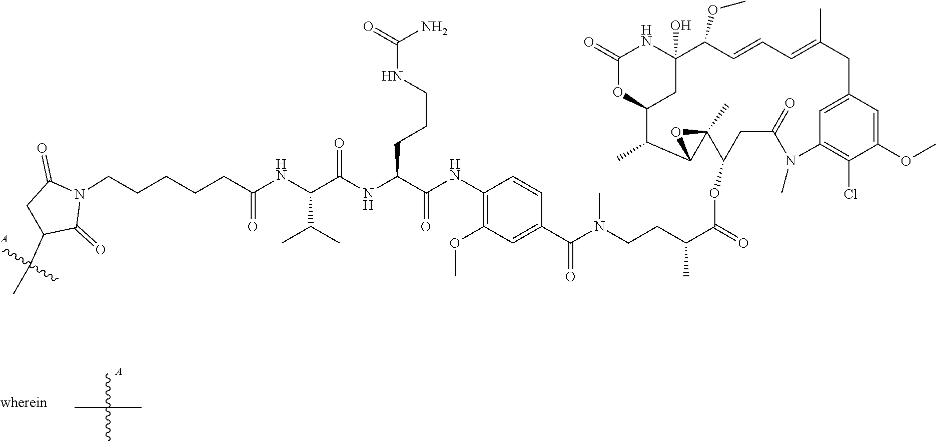

is a bond to the antibody or fragment thereof.

In some embodiments, the antibody-drug conjugate comprises an antigen binding protein, including a multispecific binding protein, comprising an anti-CD63 antigen-binding protein or antigen binding portion thereof, and

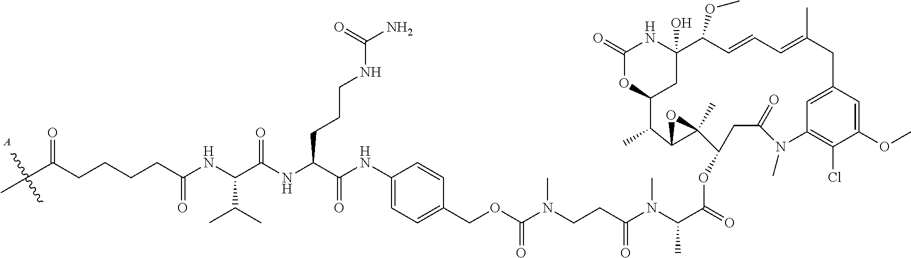

is a bond to the antibody or fragment thereof.

In some embodiments, the antibody-drug conjugate comprises an antigen binding protein, including a multispecific binding protein, comprising an anti-CD63 antigen-binding protein or antigen binding portion thereof, and

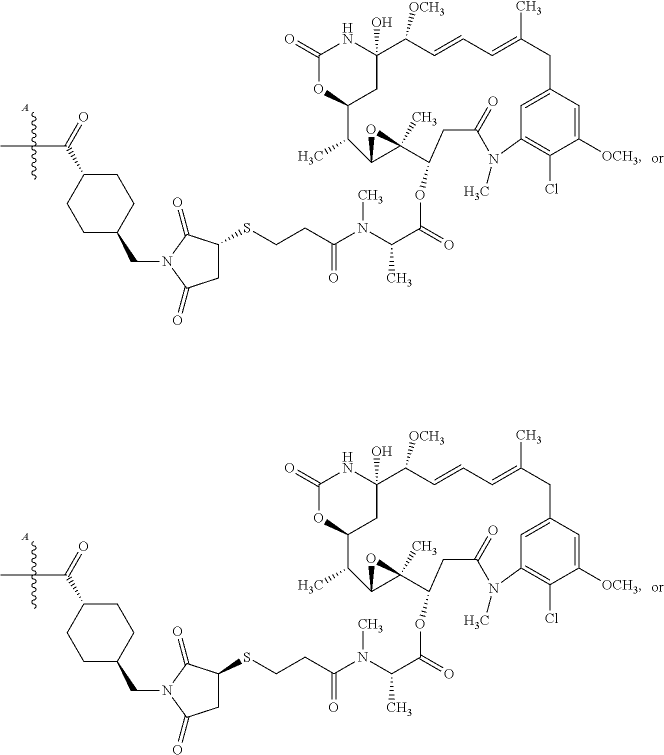

a mixture thereof,
wherein

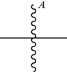

is a bond to the antibody or fragment thereof.

In some embodiments, the bond contacts the antibody or fragment thereof via a sulfur constituent of a cysteine residue.

In some embodiments, the bond contacts the antibody or fragment thereof via a nitrogen constituent of a lysine residue.

In any of the various embodiments of the antibody-drug conjugates discussed above or herein, the antibody-drug conjugate can comprise from 1 to 10 cytotoxic agents per antigen binding protein, including a multispecific binding protein, comprising an anti-CD63 antigen-binding protein or antigen binding portion thereof.

### DRAWINGS

FIG. 1 schematically represents multidomain therapeutic proteins. Panel A depicts a multidomain therapeutic protein comprising a bispecific antibody (ii) and a replacement enzyme (i). Panel B depicts an enzyme-Fc fusion polypeptide (i) associating with an internalization effector-specific half-body (ii) to form the multidomain therapeutic protein. Panel C depicts a replacement enzyme (hexagon) covalently linked to the C-terminus of the heavy chain of an anti-internalization effector antibody. Panel D depicts a replacement enzyme (hexagon) covalently linked to the N-terminus of the heavy chain of an anti-internalization effector antibody. Panel E depicts a replacement enzyme (hexagon) covalently linked to the C-terminus of the light chain of an anti-internalization effector antibody. Panel F depicts a replacement enzyme (hexagon) covalently linked to the N-terminus of the light chain of an anti-internalization effector antibody. Panel G depicts a replacement enzyme (hexagon) covalently linked to the C-terminus of a single-chain variable fragment (scFv) containing a VH region (shaded bar) and a VL region (open bar). Panel H depicts a replacement enzyme (hexagon) covalently linked to two scFv domains, the first scFv (i) which serves as a first delivery domain, and the second scFv (ii) which serves as a second delivery domain. Exemplary anti-CD63 V_{H} and V_{L} amino acid sequences (and nucleotide sequences encoding same) that may be used to construct a multidomain therapeutic protein comprising an anti-CD63 antibody or antigen binding portion thereof are provided in Table 1.

FIG. 2 is a non-limiting exemplary illustration of an AAV gene therapy vector that encodes a multidomain therapeutic protein represented in panel, wherein the scFv is an anti-human CD63 scFv and the replacement enzyme is GAA (e.g., anti-hCD63scFv::hGAA; see, e.g., the amino acid sequence set forth as SEQ ID NO: 364 wherein the anti-hCD63scFv is derived from the H4H12450N antibody. Amino acids 1-117 of SEQ ID NO: 364 provide the amino acid sequence of the heavy chain variable domain (VH) of the H4H12450N antibody; amino acids 118-132 of SEQ ID NO: 364 provide an amino acid linker sequence between the heavy and light chain variable domains of H4H12450N; amino acids 133-240 of SEQ ID NO: 364 provide the amino acid sequence of the light chain variable domain (V_{L}) of the H4H12450N antibody; amino acids 241-245of SEQ ID NO: 364 provides an amino acid linker sequence between the anti-hCD63scFv and GAA; and amino acids 246-1128 of SEQ ID NO: 364 provides the amino acid sequence of the replacement enzyme GAA, or biologically active portion thereof. Exemplary 5′ITR and 3′ ITR sequences are respectively set forth as SEQ ID NO: 365 and SEQ ID NO: 366. An exemplary liver specific enhancer (serpina 1) is set forth as SEQ ID NO: 367. An exemplary liver specific promoter (TTR) is set forth as SEQ ID NO: 368. Additional exemplary anti-CD63 VH and V_{L} amino acid sequences (and nucleotide sequences encoding same) that may be used to construct a multidomain therapeutic protein, e.g., for the treatment of Pompe disease in a patient in need thereof, comprising an anti-CD63 antibody or antigen binding portion thereof are provided in Table 1.

### DESCRIPTION

Provided herein are novel anti-human CD63 antibodies, and monovalent antigen binding fragments thereof, which are useful in mediating internalization of CD63. The anti-human CD63 antibodies, and monovalent antigen binding fragments thereof may be useful, e.g., in the treatment of diseases, as part of multispecific antigen binding protein and/or multidomain therapeutic protein, and/or as an antibody drug conjugate.

This invention is not limited to particular embodiments, compositions, methods and experimental conditions described, as such embodiments, compositions, methods and conditions may vary. The terminology used herein is for the purpose of describing particular embodiments only, and is not intended to be limiting, since the scope of the present invention will be limited only by the appended claims.

Although any methods and materials similar or equivalent to those described herein can be used in the practice or testing of the present invention, some preferred methods and materials are now described. All publications cited herein are incorporated herein by reference to describe in their entirety. Unless defined otherwise, all technical and scientific terms used herein have the same meaning as commonly understood by one of ordinary skill in the art to which this invention belongs.

Unless defined otherwise, all technical and scientific terms used herein have the same meaning as commonly understood by one of ordinary skill in the art to which this invention belongs. The term “about,” when used in reference to a particular recited numerical value, means that the value may vary from the recited value by no more than 1%. For example, the expression “about 100” includes 99 and 101 and all values in between (e.g., 99.1, 99.2, 99.3, 99.4, etc.). Although any methods and materials similar or equivalent to those described herein can be used in the practice or testing of the present invention, the preferred methods and materials are now described. All patents, applications and non-patent publications mentioned in this specification are incorporated herein by reference in their entireties.

“CD63” includes a protein that in humans is encoded by the CD63 gene and has an amino acid sequence set forth as SEQ ID NO: 337. CD63, is a member of the tetraspanin superfamily of cell surface proteins that span the cell membrane four times. CD63 is expressed in virtually all tissues and is thought to be involved in forming and stabilizing signaling complexes. CD63 localizes to the cell membrane, lysosomal membrane, and late endosomal membrane. CD63 is known to associate with integrins and may be involved in epithelial-mesenchymal transitioning. See H. Maecker et al., “The tetraspanin superfamily: molecular facilitators,” 11(6) FASEB J. 428-42, May 1997; and M. Metzelaar et al., “CD63 antigen. A novel lysosomal membrane glycoprotein, cloned by a screening procedure for intracellular antigens in eukaryotic cells,” 266 J. Biol. Chem. 3239-3245, 1991. CD63 exhibits biological activities of an internalizing effector. All references to proteins, polypeptides and protein fragments herein are intended to refer to the human version of the respective protein, polypeptide or protein fragment unless explicitly specified as being from a non-human species. Thus, the expression “CD63” means human CD63 unless specified as being from a non-human species, e.g., “mouse CD63,” “monkey CD63,” etc.

An “internalizing effector” includes a protein that is capable of being internalized into a cell or that otherwise participates in or contributes to retrograde membrane trafficking. In some instances, the internalizing effector is a protein that undergoes transcytosis; that is, the protein is internalized on one side of a cell and transported to the other side of the cell (e.g., apical-to-basal). In many embodiments, the internalizing effector protein is a cell surface-expressed protein or a soluble extracellular protein. However, the present invention also contemplates embodiments in which the internalizing effector protein is expressed within an intracellular compartment such as the endosome, endoplasmic reticulum, Golgi, lysosome, etc. For example, proteins involved in retrograde membrane trafficking (e.g., pathways from early/recycling endosomes to the trans-Golgi network) may serve as internalizing effector proteins in various embodiments of the present invention. In any event, the binding of an antibody or antigen-binding portion thereof, including multidomain therapeutic proteins and multispecific binding proteins comprising same, to an internalizing effector protein causes the entire antibody or antigen-binding portion thereof, including multidomain therapeutic proteins and multispecific binding proteins comprising same, and any molecules associated therewith (e.g., enzyme, target molecule, drug conjugate), to also become internalized into the cell. As explained below, internalizing effector proteins include proteins that are directly internalized into a cell, as well as proteins that are indirectly internalized into a cell. Internalizing effector proteins that are directly internalized into a cell include membrane-associated molecules with at least one extracellular domain (e.g., transmembrane proteins, GPI-anchored proteins, etc.), which undergo cellular internalization, and are preferably processed via an intracellular degradative and/or recycling pathway.

The phrase “an antibody that binds CD63” or an “anti-CD63 antibody” includes antibodies and antigen-binding fragments thereof that specifically recognize a single CD63 molecule. The antibodies and antigen-binding fragments of the present invention may bind soluble CD63 and/or cell surface expressed CD63. Soluble CD63 includes natural CD63 proteins as well as recombinant CD63 protein variants that lack a transmembrane domain or are otherwise unassociated with a cell membrane, for example, but not limited to a recombinant C-terminal Myc-Myc-hexahistidine (hCD63 EC loop 2-MMH; SEQ ID NO: 338) or recombinant human CD63 extracellular loop 2 expressed with a C-terminal human Fc tag (hCD63 EC loop 2-hFc; SEQ ID NO: 339).

The expression “cell surface-expressed CD63” refers to one or more CD63 protein(s) that is/are expressed on the surface of a cell in vitro or in vivo, such that at least a portion of a CD63 protein is exposed to the extracellular side of the cell membrane and is accessible to an antigen-binding portion of an antibody. “Cell surface-expressed CD63” includes CD63 proteins contained within the context of a functional T cell receptor in the membrane of a cell. A “cell surface-expressed CD63” can comprise or consist of a CD63 protein expressed on the surface of a cell which normally expresses CD63 protein. Alternatively, “cell surface-expressed CD63” can comprise or consist of CD63 protein expressed on the surface of a cell that normally does not express human CD63 on its surface but has been artificially engineered to express CD63 on its surface.

The term “antigen-binding molecule” includes antibodies and antigen-binding fragments of antibodies, including, e.g., bispecific antibodies.

The term “avidity” refers to the ability of an antigen-binding molecule to reach a threshold of target engagement in order to achieve its desired effect. The phrase “avidity-driven binding” or “avidity-driven pairing” in the context of multiple target antigens and a multispecific antigen-binding molecule refers to the mechanism of action wherein the multispecific antigen-binding molecule provides at least two monovalent binding arms, and a first binding arm (or arms) binds to a first target antigen with high affinity. A second binding arm (or arms) binds a second target antigen with low affinity such that the second binding arm does not bind the second target antigen unless both antigens are in proximity to each other, such as present on the same cell. Thus, the high affinity binding to the first target antigen increases the avidity of the low affinity arm for the second binding arm and mediates binding to the second target (Rhoden, J. J., et al., May 20, 2016, J Biol Chem. 291, 11337-11347, first published on Mar. 28, 2016 doi: 10.1074/jbc.M116.714287; Jarantow, S. W., et al., Oct. 9, 2015, J Biol Chem. 290(41):24689-704. doi: 10.1074/jbc.M115.651653. Epub 2015 Aug. 10).

The term “antibody” refers to any antigen-binding molecule or molecular complex comprising at least one complementarity determining region (CDR) that specifically binds to or interacts with a particular antigen (e.g., CD63). The term “antibody”, as used herein, includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3. The term “high affinity” antibody refers to those antibodies having a binding affinity to their target of at least 10⁻⁹ M, at least 10⁻¹⁰ M; at least 10⁻¹¹ M; or at least 10⁻¹² M, as measured by surface plasmon resonance, e.g., BIACORE™ or solution-affinity ELISA. The term “antibody” may encompass any type of antibody, such as e.g. monoclonal or polyclonal. Moreover, the antibody may be or any origin, such as e.g. mammalian or non-mammalian. In one embodiment, the antibody may be mammalian or avian. In a further embodiment, the antibody may be or human origin and may further be a human monoclonal antibody.

The term “antibody” also includes antigen-binding fragments of full antibody molecules. The terms “antigen-binding portion” of an antibody, “antigen-binding fragment” of an antibody, and the like include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment”.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of the present invention using routine techniques available in the art.

The antibodies of the present invention may function through complement-dependent cytotoxicity (CDC) or antibody-dependent cell-mediated cytotoxicity (ADCC). “Complement-dependent cytotoxicity” (CDC) refers to lysis of antigen-expressing cells by an antibody of the invention in the presence of complement. “Antibody-dependent cell-mediated cytotoxicity” (ADCC) refers to a cell-mediated reaction in which nonspecific cytotoxic cells that express Fc receptors (FcRs) (e.g., Natural Killer (NK) cells, neutrophils, and macrophages) recognize bound antibody on a target cell and thereby lead to lysis of the target cell. CDC and ADCC can be measured using assays that are well known and available in the art. (See, e.g., U.S. Pat. Nos. 5,500,362 and 5,821,337, and Clynes et al. (1998) Proc. Natl. Acad. Sci. (USA) 95:652-656). The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

In certain embodiments of the invention, the anti-CD63 antibodies of the invention are human antibodies. The term “human antibody” refers to antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody” is not intended to include antibodies in which CDR sequences derived from the germline of another mammalian species, such as a mouse, have been grafted onto human framework sequences.

The antibodies of the invention may, in some embodiments, be recombinant human antibodies. The term “recombinant human antibody” is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The instant invention encompasses antibodies having one or more mutations in the hinge, C_{H}2 or C_{H}3 region which may be desirable, for example, in production, to improve the yield of the desired antibody form.

The antibodies of the invention may be isolated antibodies. An “isolated antibody” refers to an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present invention. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The present invention also includes one-arm antibodies that bind CD63. The term “one-arm antibody” refers to an antigen-binding molecule comprising a single antibody heavy chain and a single antibody light chain. The one-arm antibodies of the present invention may comprise any of the HCVR/LCVR or CDR amino acid sequences as set forth in Table 1.

The anti-CD63 antibodies disclosed herein may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present invention includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present invention.

The present invention also includes anti-CD63 antibodies comprising variants of any of the HCVR, LCVR, and/or CDR amino acid sequences disclosed herein having one or more conservative substitutions. For example, the present invention includes anti-CD63 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences set forth in Table 1 herein.

The phrase “bispecific antibody” includes an antibody capable of selectively binding two or more epitopes. Bispecific antibodies generally comprise two different heavy chains, with each heavy chain specifically binding a different epitope-either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions, and such sequences can be expressed in a cell that expresses an immunoglobulin light chain. A typical bispecific antibody has two heavy chains each having three heavy chain CDRs, followed by (N-terminal to C-terminal) a C_{H}1 domain, a hinge, a C_{H}2 domain, and a C_{H}3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding or one or both of the heavy chains to one or both epitopes.

The phrase “heavy chain,” or “immunoglobulin heavy chain” includes an immunoglobulin heavy chain constant region sequence from any organism, and unless otherwise specified includes a heavy chain variable domain. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a CH1 domain, a hinge, a CH2 domain, and a CH3 domain. A functional fragment of a heavy chain includes a fragment that is capable of specifically recognizing an antigen (e.g., recognizing the antigen with a KD in the micromolar, nanomolar, or picomolar range), that is capable of expressing and secreting from a cell, and that comprises at least one CDR.

The phrase “light chain” includes an immunoglobulin light chain constant region sequence from any organism, and unless otherwise specified includes human kappa and lambda light chains. Light chain variable (VL) domains typically include three light chain CDRs and four framework (FR) regions, unless otherwise specified. Generally, a full-length light chain includes, from amino terminus to carboxyl terminus, a VL domain that includes FR1-CDR1-FR2-CDR2-FR3-CDR3-FR4, and a light chain constant domain. Light chains that can be used with this invention include e.g., those, that do not selectively bind either the first or second antigen selectively bound by the antigen-binding protein. Suitable light chains include those that can be identified by screening for the most commonly employed light chains in existing antibody libraries (wet libraries or in silico), where the light chains do not substantially interfere with the affinity and/or selectivity of the antigen-binding domains of the antigen-binding proteins. Suitable light chains include those that can bind one or both epitopes that are bound by the antigen-binding regions of the antigen-binding protein.

The phrase “variable domain” includes an amino acid sequence of an immunoglobulin light or heavy chain (modified as desired) that comprises the following amino acid regions, in sequence from N-terminal to C-terminal (unless otherwise indicated): FR1, CDR1, FR2, CDR2, FR3, CDR3, FR4. A “variable domain” includes an amino acid sequence capable of folding into a canonical domain (VH or VL) having a dual beta sheet structure wherein the beta sheets are connected by a disulfide bond between a residue of a first beta sheet and a second beta sheet.

The phrase “complementarity determining region,” or the term “CDR,” includes an amino acid sequence encoded by a nucleic acid sequence of an organism's immunoglobulin genes that normally (i.e., in a wildtype animal) appears between two framework regions in a variable region of a light or a heavy chain of an immunoglobulin molecule (e.g., an antibody or a T cell receptor). A CDR can be encoded by, for example, a germline sequence or a rearranged or unrearranged sequence, and, for example, by a naive or a mature B cell or a T cell. In some circumstances (e.g., for a CDR3), CDRs can be encoded by two or more sequences (e.g., germline sequences) that are not contiguous (e.g., in an unrearranged nucleic acid sequence) but are contiguous in a B cell nucleic acid sequence, e.g., as the result of splicing or connecting the sequences (e.g., V-D-J recombination to form a heavy chain CDR3).

The term “antibody fragment”, refers to one or more fragments of an antibody that retain the ability to specifically bind to an antigen. Examples of binding fragments encompassed within the term “antibody fragment” include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) Nature 241:544-546), which consists of a VH domain, (vi) an isolated CDR, and (vii) an scFv, which consists of the two domains of the Fv fragment, VL and VH, joined by a synthetic linker to form a single protein chain in which the VL and VH regions pair to form monovalent molecules. Other forms of single chain antibodies, such as diabodies are also encompassed under the term “antibody” (see e.g., Holliger et al. (1993) PNAS USA 90:6444-6448; Poljak et al. (1994) Structure 2:1121-1123).

The phrase “Fc-containing protein” includes antibodies, bispecific antibodies, immunoadhesins, and other binding proteins that comprise at least a functional portion of an immunoglobulin CH2 and CH3 region. A “functional portion” refers to a CH2 and CH3 region that can bind a Fc receptor (e.g., an FcγR; or an FcRn, i.e., a neonatal Fc receptor), and/or that can participate in the activation of complement. If the CH2 and CH3 region contains deletions, substitutions, and/or insertions or other modifications that render it unable to bind any Fc receptor and also unable to activate complement, the CH2 and CH3 region is not functional.

Fc-containing proteins can comprise modifications in immunoglobulin domains, including where the modifications affect one or more effector function of the binding protein (e.g., modifications that affect FcγR binding, FcRn binding and thus half-life, and/or CDC activity). Such modifications include, but are not limited to, the following modifications and combinations thereof, with reference to EU numbering of an immunoglobulin constant region: 238, 239, 248, 249, 250, 252, 254, 255, 256, 258, 265, 267, 268, 269, 270, 272, 276, 278, 280, 283, 285, 286, 289, 290, 292, 293, 294, 295, 296, 297, 298, 301, 303, 305, 307, 308, 309, 311, 312, 315, 318, 320, 322, 324, 326, 327, 328, 329, 330, 331, 332, 333, 334, 335, 337, 338, 339, 340, 342, 344, 356, 358, 359, 360, 361, 362, 373, 375, 376, 378, 380, 382, 383, 384, 386, 388, 389, 398, 414, 416, 419, 428, 430, 433, 434, 435, 437, 438, and 439.

For example, and not by way of limitation, the binding protein is an Fc-containing protein and exhibits enhanced serum half-life (as compared with the same Fc-containing protein without the recited modification(s)) and have a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at 428 and/or 433 (e.g., L/R/SI/P/Q or K) and/or 434 (e.g., H/F or Y); or a modification at 250 and/or 428; or a modification at 307 or 308 (e.g., 308F, V308F), and 434. In another example, the modification can comprise a 428L (e.g., M428L) and 434S (e.g., N434S) modification; a 428L, 2591 (e.g., V259I), and a 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); a 307 and/or 308 modification (e.g., 308F or 308P).

The term “antigen-binding protein,” as used herein, refers to a polypeptide or protein (one or more polypeptides complexed in a functional unit) that specifically recognizes an epitope on an antigen, such as a cell-specific antigen and/or a target antigen of the present invention. An antigen-binding protein may be multi-specific. The term “multi-specific” with reference to an antigen-binding protein means that the protein recognizes different epitopes, either on the same antigen or on different antigens. A multi-specific antigen-binding protein of the present invention can be a single multifunctional polypeptide, or it can be a multimeric complex of two or more polypeptides that are covalently or non-covalently associated with one another. The term “antigen-binding protein” includes antibodies or fragments thereof of the present invention that may be linked to or co-expressed with another functional molecule, e.g., another peptide or protein. For example, an antibody or fragment thereof can be functionally linked (e.g., by chemical coupling, genetic fusion, non-covalent association or otherwise) to one or more other molecular entities, such as a protein or fragment thereof to produce a bispecific or a multi-specific antigen-binding molecule with a second binding specificity.

“Multidomain therapeutic protein” includes (i) a single protein that contains more than one functional domain, (ii) a protein that contains more than one polypeptide chain, and (iii) a mixture of more than one protein or more than one polypeptide. The term polypeptide is generally taken to mean a single chain of amino acids linked together via peptide bonds. The term protein encompasses the term polypeptide, but also includes more complex structures. That is, a single polypeptide is a protein, and a protein can contain one or more polypeptides associated in a higher order structure. For example, hemoglobin is a protein containing four polypeptides: two alpha globin polypeptides and two beta globin polypeptides. Myoglobin is also a protein, but it contains only a single myoglobin polypeptide.

The multidomain therapeutic protein comprises one or more polypeptide(s) and at least two domains providing two functions. One of those domains is the “enzyme domain” which provides the replacement of a defective protein activity associated with an enzyme deficiency disease. The other of those domains is the “delivery domain” which provides binding to an internalization effector, e.g., CD63, such as an anti-CD63 antibody as disclosed herein or antigen-binding fragment thereof. Thus, a single polypeptide that provides an enzyme replacement activity and the ability to bind to an internalization effector (a.k.a. internalization effector-binding protein (delivery domain activity) is a multidomain therapeutic protein. Also, a mixture of proteins, wherein one protein provides the enzyme function, and another protein provides the internalization effector binding activity, is a multidomain therapeutic protein

The term “protein” means any amino acid polymer having more than about 20 amino acids covalently linked via amide bonds. Proteins contain one or more amino acid polymer chains, generally known in the art as “polypeptides”. Thus, a polypeptide may be a protein, and a protein may contain multiple polypeptides to form a single functioning biomolecule. Disulfide bridges (i.e., between cysteine residues to form cystine) may be present in some proteins. These covalent links may be within a single polypeptide chain, or between two individual polypeptide chains. For example, disulfide bridges are essential to proper structure and function of insulin, immunoglobulins, protamine, and the like. For a recent review of disulfide bond formation, see Oka and Bulleid, “Forming disulfides in the endoplasmic reticulum,” 1833(11) Biochim Biophys Acta 2425-9 (2013).

As used herein, “protein” includes biotherapeutic proteins, recombinant proteins used in research or therapy, trap proteins and other Fc-fusion proteins, chimeric proteins, antibodies, monoclonal antibodies, human antibodies, bispecific antibodies, antibody fragments, nanobodies, recombinant antibody chimeras, scFv fusion proteins, cytokines, chemokines, peptide hormones, and the like. Proteins may be produced using recombinant cell-based production systems, such as the insect baculovirus system, yeast systems (e.g., Pichia sp.), mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation,” 28 Biotechnol Genet Eng Rev. 147-75 (2012).

As used herein, the term “epitope” refers to the portion of the antigen which is recognized by the multi-specific antigen-binding polypeptide. A single antigen (such as an antigenic polypeptide) may have more than one epitope. Epitopes may be defined as structural or functional. Functional epitopes are generally a subset of structural epitopes and are defined as those residues that directly contribute to the affinity of the interaction between the antigen-binding polypeptide and the antigen. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics. Epitopes formed from contiguous amino acids are typically retained on exposure to denaturing solvents, whereas epitopes formed by tertiary folding are typically lost on treatment with denaturing solvents.

The term “domain” refers to any part of a protein or polypeptide having a particular function or structure. Preferably, domains of the present invention bind to cell-specific or target antigens. Cell-specific antigen- or target antigen-binding domains, and the like, as used herein, include any naturally occurring, enzymatically obtainable, synthetic, or genetically engineered polypeptide or glycoprotein that specifically binds an antigen.

The term “half-body” or “half-antibody”, which are used interchangeably, refers to half of an antibody, which essentially contains one heavy chain and one light chain. Antibody heavy chains can form dimers, thus the heavy chain of one half-body can associate with heavy chain associated with a different molecule (e.g., another half-body) or another Fc-containing polypeptide. Two slightly different Fc-domains may “heterodimerize” as in the formation of bispecific antibodies or other heterodimers, -trimers, -tetramers, and the like. See Vincent and Murini, “Current strategies in antibody engineering: Fc engineering and pH-dependent antigen binding, bispecific antibodies and antibody drug conjugates,” 7 Biotechnol. J. 1444-1450 (20912); and Shimamoto et al., “Peptibodies: A flexible alternative format to antibodies,” 4(5) MAbs 586-91 (2012).

In one embodiment, the half-body variable domain specifically recognizes the internalization effector and the half body Fc-domain dimerizes with an Fc-fusion protein that comprises a replacement enzyme (e.g., a peptibody) Id, 586.

The term “single-chain variable fragment” or “scFv” includes a single chain fusion polypeptide containing an immunoglobulin heavy chain variable region (VH) and an immunoglobulin light chain variable region (VL). In some embodiments, the VH and VL are connect by a linker sequence of 10 to 25 amino acids. ScFv polypeptides may also include other amino acid sequences, such as CL or CH1 regions. ScFv molecules can be manufactured by phage display or made by directly subcloning the heavy and light chains from a hybridoma or B-cell. Ahmad et al., Clinical and Developmental Immunology, volume 2012, article ID 98025 is incorporated herein by reference for methods of making scFv fragments by phage display and antibody domain cloning.

### Bispecific Antigen-Binding Molecules

The anti-CD63 antibodies and antigen-binding fragments thereof (including multispecific antigen-binding molecules and multidomain therapeutic proteins comprising anti-CD63 antibodies or antigen-binding fragments thereof) of the present invention may be monospecific, bi-specific, or multispecific. Multispecific antibodies may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244. The anti-CD63 antibodies and antigen-binding fragments thereof (including multispecific antigen-binding molecules and multidomain therapeutic proteins comprising anti-CD63 antibodies or antigen-binding fragments thereof) of the present invention can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multispecific antibody with a second or additional binding specificity.

Use of the expression “anti-CD63 antibody” herein is intended to include both monospecific anti-CD63 antibodies as well as bispecific antibodies comprising a CD63-binding arm and a “target”-binding arm. Thus, the present invention includes bispecific antibodies wherein one arm of an immunoglobulin binds human CD63, and the other arm of the immunoglobulin is specific for another target molecule. The CD63-binding arm can comprise any of the HCVR/LCVR or CDR amino acid sequences as set forth in Table 1 herein.

In certain embodiments, the CD63-binding arm binds to human CD63 and induces internalization of the CD63 and antibody bound thereto. In certain embodiments, the CD63-binding arm binds weakly to human CD63 and induces internalization of CD63 and antibody bound thereto. In other embodiments, the CD63-binding arm binds weakly to human CD63 and induces tumor-associated antigen-expressing cell killing in the context of a bispecific or multispecific antibody, e.g., comprising a CD63-binding arm and a “target”-binding arm, where the “target”-binding arm is a tumor associated antigen (TAA). Non-limiting examples of specific tumor-associated antigens include, e.g., AFP, ALK, BAGE proteins, β-catenin, brc-abl, BRCA1, BORIS, CA9, carbonic anhydrase IX, caspase-8, CD40, CDK4, CEA, CTLA4, cyclin-B1, CYP1B1, EGFR, EGFRvIII, ErbB2/Her2, ErbB3, ErbB4, ETV6-AML, EphA2, Fra-1, FOLR1, GAGE proteins (e.g., GAGE-1, -2), GD2, GD3, GloboH, glypican-3, GM3, gp100, Her2, HLA/B-raf, HLA/k-ras, HLA/MAGE-A3, hTERT, LMP2, MAGE proteins (e.g., MAGE-1, -2, -3, -4, -6, and -12), MART-1, mesothelin, ML-IAP, Muc1, Muc16 (CA-125), MUM1, NA17, NY-BR1, NY-BR62, NY-BR85, NY-ESO1, OX40, p15, p53, PAP, PAX3, PAX5, PCTA-1, PLAC1, PRLR, PRAME, PSMA (FOLH1), RAGE proteins, Ras, RGS5, Rho, SART-1, SART-3, Steap-1, Steap-2, survivin, TAG-72, TGF-β, TMPRSS2, Tn, TRP-1, TRP-2, tyrosinase, and uroplakin-3. In other embodiments, the CD63-binding arm binds or associated weakly with human and cynomolgus (monkey) CD63, yet the binding interaction is not detectable by in vitro assays known in the art.

In certain exemplary embodiments of the present invention, the bispecific antigen-binding molecule is a bispecific antibody. Each antigen-binding domain of a bispecific antibody comprises a heavy chain variable domain (HCVR) and a light chain variable domain (LCVR). In the context of a bispecific antigen-binding molecule comprising a first and a second antigen-binding domain (e.g., a bispecific antibody), the CDRs of the first antigen-binding domain may be designated with the prefix “A1” and the CDRs of the second antigen-binding domain may be designated with the prefix “A2”. Thus, the CDRs of the first antigen-binding domain may be referred to herein as A1-HCDR1, A1-HCDR2, and A1-HCDR3; and the CDRs of the second antigen-binding domain may be referred to herein as A2-HCDR1, A2-HCDR2, and A2-HCDR3.

The first antigen-binding domain and the second antigen-binding domain may be directly or indirectly connected to one another to form a bispecific antigen-binding molecule of the present invention. Alternatively, the first antigen-binding domain and the second antigen-binding domain may each be connected to a separate multimerizing domain. The association of one multimerizing domain with another multimerizing domain facilitates the association between the two antigen-binding domains, thereby forming a bispecific antigen-binding molecule. A “multimerizing domain” is any macromolecule, protein, polypeptide, peptide, or amino acid that has the ability to associate with a second multimerizing domain of the same or similar structure or constitution. For example, a multimerizing domain may be a polypeptide comprising an immunoglobulin C_{H}3 domain. A non-limiting example of a multimerizing component is an Fc portion of an immunoglobulin (comprising a C_{H}2-C_{H}3 domain), e.g., an Fc domain of an IgG selected from the isotypes IgG1, IgG2, IgG3, and IgG4, as well as any allotype within each isotype group.

Bispecific antigen-binding molecules of the present invention will typically comprise two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

In certain embodiments, the multimerizing domain is an Fc fragment or an amino acid sequence of from 1 to about 200 amino acids in length containing at least one cysteine residue. In other embodiments, the multimerizing domain is a cysteine residue, or a short cysteine-containing peptide. Other multimerizing domains include peptides or polypeptides comprising or consisting of a leucine zipper, a helix-loop motif, or a coiled-coil motif.

Any bispecific antibody format or technology may be used to make the bispecific antigen-binding molecules of the present invention. For example, an antibody or fragment thereof having a first antigen binding specificity can be functionally linked (e.g., by chemical coupling, genetic fusion, noncovalent association or otherwise) to one or more other molecular entities, such as another antibody or antibody fragment having a second antigen-binding specificity to produce a bispecific antigen-binding molecule. Specific exemplary bispecific formats that can be used in the context of the present invention include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mab² bispecific formats (see, e.g., Klein et al. 2012, mAbs 4:6, 1-11, and references cited therein, for a review of the foregoing formats).

In the context of bispecific antigen-binding molecules of the present invention, the multimerizing domains, e.g., Fc domains, may comprise one or more amino acid changes (e.g., insertions, deletions or substitutions) as compared to the wild-type, naturally occurring version of the Fc domain. For example, the invention includes bispecific antigen-binding molecules comprising one or more modifications in the Fc domain that results in a modified Fc domain having a modified binding interaction (e.g., enhanced or diminished) between Fc and FcRn. In one embodiment, the bispecific antigen-binding molecule comprises a modification in a C_{H}2 or a C_{H}3 region, wherein the modification increases the affinity of the Fc domain to FcRn in an acidic environment (e.g., in an endosome where pH ranges from about 5.5 to about 6.0). Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P).

The present invention also includes bispecific antigen-binding molecules comprising a first C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). See, for example, U.S. Pat. No. 8,586,713. Further modifications that may be found within the second C_{H}3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies.

In certain embodiments, the Fc domain may be chimeric, combining Fc sequences derived from more than one immunoglobulin isotype. For example, a chimeric Fc domain can comprise part or all of a C_{H}2 sequence derived from a human IgG1, human IgG2 or human IgG4 C_{H}2 region, and part or all of a C_{H}3 sequence derived from a human IgG1, human IgG2 or human IgG4. A chimeric Fc domain can also contain a chimeric hinge region. For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region. A particular example of a chimeric Fc domain that can be included in any of the antigen-binding molecules set forth herein comprises, from N- to C-terminus: [IgG4 C_{H}1]-[IgG4 upper hinge]-[IgG2 lower hinge]-[IgG4 C_{H}2]-[IgG4 C_{H}3]. Another example of a chimeric Fc domain that can be included in any of the antigen-binding molecules set forth herein comprises, from N- to C-terminus: [IgG1 C_{H}1]-[IgG1 upper hinge]-[IgG2 lower hinge]-[IgG4 CH2]-[IgG1 CH3]. These and other examples of chimeric Fc domains that can be included in any of the antigen-binding molecules of the present invention are described in US Publication 2014/0243504, published Aug. 28, 2014, which is herein incorporated in its entirety. Chimeric Fc domains having these general structural arrangements, and variants thereof, can have altered Fc receptor binding, which in turn affects Fc effector function.

In certain embodiments, the invention provides an antibody heavy chain wherein the heavy chain constant region (CH) region comprises an amino acid sequence at least 95%, at least 96%, at least 97%, at least 98%, at least 99% identical to any one of SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, or SEQ ID NO: 351. In some embodiments, the heavy chain constant region (CH) region comprises an amino acid sequence selected from the group consisting of SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, or SEQ ID NO: 351.

In other embodiments, the invention provides an antibody heavy chain wherein the Fc domain comprises an amino acid sequence at least 95%, at least 96%, at least 97%, at least 98%, at least 99% identical to any one of SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, or SEQ ID NO: 363. In some embodiments, the Fc domain comprises an amino acid sequence selected form the group consisting of SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, or SEQ ID NO: 363.

### Multidomain Therapeutic Proteins

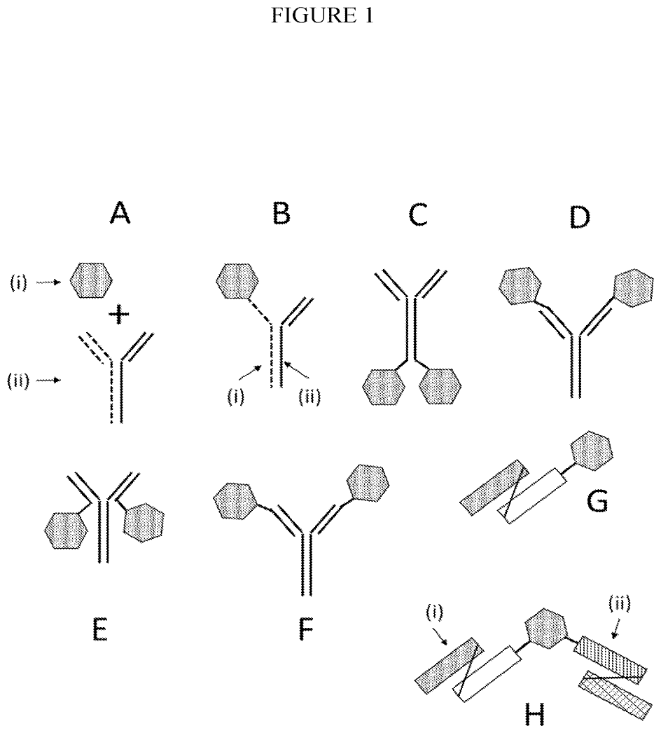

FIG. 1 depicts various exemplars of multidomain therapeutic proteins. In one example (FIG. 1, panel A), the multidomain therapeutic protein contains an enzyme (represented by the hexagon) and a bispecific antibody (the IE-BP) that binds the enzyme (hashed lines) and an internalization effector (solid lines). Here, one arm of the bispecific antibody binds non-covalently to the enzyme, and the other arm binds non-covalently to the internalization effector, thereby enabling the internalization of the replacement enzyme into the cell or subcellular compartment. In another example (panel B), the multidomain therapeutic protein comprises a single protein containing two polypeptides, one polypeptide having enzyme function and the other having delivery domain function. Here, the enzyme is fused to an immunoglobulin Fc domain or heavy chain constant region, which associates with the Fc domain of the enzyme half-antibody to form the bifunctional multidomain therapeutic protein. The embodiment depicted in panel B is similar to that in panel A, except that the enzyme is covalently attached to one of the half-antibodies, rather than through antigen-antibody interaction at the immunoglobulin variable domain of the half-antibody.

In other examples, the multidomain therapeutic protein consists of the enzyme covalently linked (directly or indirectly through a linker) to the delivery domain. In one embodiment, the enzyme is attached to the C-terminus of an immunoglobulin molecule (e.g., the heavy chain or alternatively the light chain). In another embodiment, the enzyme is attached to the N-terminus of the immunoglobulin molecule (e.g., the heavy chain or alternatively the light chain). In these exemplars, the immunoglobulin molecule is the delivery domain. In yet another embodiment, the enzyme is attached to the C-terminus of a scFv molecule that binds the internalization effector.

In one embodiment, the multidomain therapeutic protein comprises two delivery domains. In one embodiment, the first delivery domain binds to a lysosomal trafficking molecule or other internalization effector (e.g., CD63). In another embodiment, the second delivery domain binds to a transcytosis effector to facilitate transcellular transport of the multidomain therapeutic protein. In one embodiment, the transcytosis effector is inter alia an LDL receptor, an IgA receptor, a transferrin receptor, or a neonatal Fc receptor (FcRn). In a specific embodiment, the transcytosis delivery domain comprises a molecule that binds to a transferrin receptor, such as e.g., an anti-transferrin receptor antibody or an anti-transferrin receptor scFv molecule. Tuma and Hubbard, “Transcytosis: Crossing Cellular Barriers,” Physiological Reviews, 83(3): 871-935 (1 Jul. 2003) is incorporated herein by reference for cell surface receptors that mediate transcytosis that are useful in the practice of the subject invention.

“Enzyme domain” or “enzyme” denotes any protein associated with the etiology or physiological effect of an enzyme deficiency disease. An enzyme includes the actual enzyme, transport protein, receptor, or other protein that is defective and which is attributed as the molecular lesion that caused the disease. An enzyme also includes any protein that can provide a similar or sufficient biochemical or physiological activity that replaces or circumvents the molecular lesion of the disease. For example, an “isozyme” may be used as an enzyme.

In some embodiments, the enzyme is a hydrolase, including esterases, glycosylases, hydrolases that act on ether bonds, peptidases, linear amidases, diphosphatases, ketone hydrolases, halogenases, phosphoamidases, sulfohydrolases, sulfinases, desulfinases, and the like. In some embodiments, the enzyme is a glycosylase, including glycosidases and N-glycosylases. In some embodiments, the enzyme is a glycosidase, including alpha-amylase, beta-amylase, glucan 1,4-alpha-glucosidase, cellulose, endo-1,3(4)-beta-glucanase, inulinase, endo-1,4-beta-xylanase, endo-1,4-b-xylanase, dextranase, chitinase, polygalacturonidase, lysozyme, exo-alpha-sialidase, alpha-glucosidase, beta-glucosidase, alpha-galactosidase, beta-galactosidase, alpha-mannosidase, beta-mannosidase, beta-fructofuranosidase, alpha,alpha-trehalose, beta-glucuronidase, xylan endo-1,3-beta-xylosidase, amylo-alpha-1,6-glucosidase, hyaluronoglucosaminidase, hyaluronoglucuronidase, and the like.

In the case of Pompe disease, in which the molecular defect is a defect in a-glucosidase activity, enzymes include human alpha-glucosidase (GAA), and “isozymes” such as other alpha-glucosidases, engineered recombinant alpha-glucosidase, other glucosidases, recombinant glucosidases, any protein engineered to hydrolyze a terminal non-reducing 1-4 linked alpha-glucose residue to release a single alpha-glucose molecule, any EC 3.2.1.20 enzyme, natural or recombinant low pH carbohydrate hydrolases for glycogen or starches, and glucosyl hydrolases such as sucrase isomaltase, maltase glucoamylase, glucosidase II, and neutral alpha-glucosidase. An exemplary gene therapy vector

### Germline Mutations

The anti-CD63 antibodies disclosed herein may comprise one or more amino acid substitutions, insertions and/or deletions in the framework and/or CDR regions of the heavy chain variable domains as compared to the corresponding germline sequences from which the antibodies were derived.

The present invention also includes anti-CD63 antibodies and antigen-binding fragments thereof (including multispecific antigen-binding molecules and multidomain therapeutic proteins comprising anti-CD63 antibodies or antigen-binding fragments thereof) of the present invention which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”), and having weak or no detectable binding to a CD63 antigen. Several such exemplary antibodies that recognize CD63 are described in Table 1 herein.

Furthermore, the anti-CD63 antibodies and antigen-binding fragments thereof (including multispecific antigen-binding molecules and multidomain therapeutic proteins comprising anti-CD63 antibodies or antigen-binding fragments thereof) of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be tested for one or more desired properties such as, improved binding specificity, weak or reduced binding affinity, improved or enhanced pharmacokinetic properties, reduced immunogenicity, etc. Antibodies and antigen-binding fragments obtained in this general manner given the guidance of the present disclosure are encompassed within the present invention.

The present invention also includes anti-CD63 antibodies and antigen-binding fragments thereof (including multispecific antigen-binding molecules and multidomain therapeutic proteins comprising anti-CD63 antibodies or antigen-binding fragments thereof) comprising variants of any of the HCVR, LCVR, and/or CDR amino acid sequences disclosed herein having one or more conservative substitutions. For example, the present invention includes anti-CD63 antibodies and antigen-binding fragments thereof (including multispecific antigen-binding molecules and multidomain therapeutic proteins comprising anti-CD63 antibodies or antigen-binding fragments thereof) having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences set forth in Table 1 herein. The antibodies, antigen-binding fragments thereof, multispecific antigen-binding molecules, and multidomain therapeutic proteins of the present invention may comprise one or more amino acid substitutions, insertions and/or deletions in the framework and/or CDR regions of the heavy and light chain variable domains as compared to the corresponding germline sequences from which the individual antigen-binding domains were derived, while maintaining or improving the desired weak-to-no detectable binding to, e.g., CD63. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein, i.e. the amino acid substitution maintains or improves the desired weak to no detectable binding affinity in the case of anti-CD63 binding molecules. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445. A “moderately conservative” replacement is any change having a nonnegative value in the PAM250 log-likelihood matrix.

The present invention also includes anti-CD63 antibodies and antigen-binding fragments thereof (including multispecific antigen-binding molecules and multidomain therapeutic proteins comprising anti-CD63 antibodies or antigen-binding fragments thereof) comprising an antigen-binding domain with an HCVR and/or CDR amino acid sequence that is substantially identical to any of the HCVR and/or CDR amino acid sequences disclosed herein, while maintaining or improving the desired weak affinity to CD63 antigen. The term “substantial identity” or “substantially identical,” when referring to an amino acid sequence means that two amino acid sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402.

Once obtained, antigen-binding domains that contain one or more germline mutations were tested for decreased binding affinity utilizing one or more in vitro assays. Although antibodies that recognize a particular antigen are typically screened for their purpose by testing for high (i.e. strong) binding affinity to the antigen, the antibodies of the present invention exhibit weak binding or no detectable binding. Bispecific antigen-binding molecules comprising one or more antigen-binding domains obtained in this general manner are also encompassed within the present invention and were found to be advantageous as avidity-driven tumor therapies.

Unexpected benefits, for example, improved pharmacokinetic properties and low toxicity to the patient may be realized from further modifying the antibodies of the invention by the methods described herein.

### Binding Properties of the Antibodies

The term “binding” in the context of the binding of an antibody, immunoglobulin, antibody-binding fragment, or Fc-containing protein to either, e.g., a predetermined antigen, such as a cell surface protein or fragment thereof, typically refers to an interaction or association between a minimum of two entities or molecular structures, such as an antibody-antigen interaction.

For instance, binding affinity typically corresponds to a K_{D} value of about 10⁻⁷ M or less, such as about 10⁻⁸ M or less, such as about 10⁻⁹ M or less when determined by, for instance, surface plasmon resonance (SPR) technology in a BIAcore 3000 instrument using the antigen as the ligand and the antibody, Ig, antibody-binding fragment, or Fc-containing protein as the analyte (or antiligand). Cell-based binding strategies, such as fluorescent-activated cell sorting (FACS) binding assays, are also routinely used and provide binding characterization data with respect to cell-surface expressed proteins. FACS data correlates well with other methods such as radioligand competition binding and SPR (Benedict, CA, J Immunol Methods. 1997, 201(2):223-31; Geuijen, C A, et al. J Immunol Methods. 2005, 302(1-2):68-77).

Accordingly, the anti-CD63 antibodies and antigen-binding fragments thereof (including multispecific antigen-binding molecules and multidomain therapeutic proteins comprising anti-CD63 antibodies or antigen-binding fragments thereof) of the invention binds to the predetermined antigen or cell surface molecule (receptor) having an affinity corresponding to a K_{D} value that is at least ten-fold lower than its affinity for binding to a non-specific antigen (e.g., BSA, casein). According to the present invention, the affinity of an antibody corresponding to a K_{D} value that is equal to or less than ten-fold lower than a non-specific antigen may be considered non-detectable binding, however such an antibody may be paired with a second antigen binding arm for the production of a bispecific antibody of the invention.

The term “K_{D}” or “KD” in molar (M) refers to the dissociation equilibrium constant of a particular antibody-antigen interaction, or the dissociation equilibrium constant of an antibody or antibody-binding fragment binding to an antigen. There is an inverse relationship between K_{D} and binding affinity, therefore the smaller the K_{D} value, the higher, i.e. stronger, the affinity. Thus, the terms “higher affinity” or “stronger affinity” relate to a higher ability to form an interaction and therefore a smaller K_{D} value, and conversely the terms “lower affinity” or “weaker affinity” relate to a lower ability to form an interaction and therefore a larger K_{D} value. In some circumstances, a higher binding affinity (or K_{D}) of a particular molecule (e.g. antibody) to its interactive partner molecule (e.g. antigen X) compared to the binding affinity of the molecule (e.g. antibody) to another interactive partner molecule (e.g. antigen Y) may be expressed as a binding ratio determined by dividing the larger K_{D} value (lower, or weaker, affinity) by the smaller K_{D} (higher, or stronger, affinity), for example expressed as 5-fold or 10-fold greater binding affinity, as the case may be.

In some embodiments, a bispecific antigen-binding molecule of the invention, or conjugate thereof, binds to a target molecule with a binding affinity (K_{D} value) greater than 10-fold its binding affinity to CD63. As such, the bispecific molecule has a much stronger binding affinity to the target molecule than its binding affinity to CD63. In some cases, the binding affinity is measured by a surface plasmon resonance assay at 37° C., or equivalent assay.

The term “k_{d}” (see −1 or 1/s) refers to the dissociation rate constant of a particular antibody-antigen interaction, or the dissociation rate constant of an antibody or antibody-binding fragment. Said value is also referred to as the k_{off} value.

The term “kₐ” (M-1 x sec-1 or 1/M) refers to the association rate constant of a particular antibody-antigen interaction, or the association rate constant of an antibody or antibody-binding fragment.

The term “KA” (M-1 or 1/M) refers to the association equilibrium constant of a particular antibody-antigen interaction, or the association equilibrium constant of an antibody or antibody-binding fragment. The association equilibrium constant is obtained by dividing the kₐ by the k_{d}.

The term “EC50” or “EC₅₀” refers to the half maximal effective concentration, which includes the concentration of an antibody which induces a response halfway between the baseline and maximum after a specified exposure time. The EC₅₀ essentially represents the concentration of an antibody where 50% of its maximal effect is observed. In certain embodiments, the EC₅₀ value equals the concentration of an antibody of the invention that gives half-maximal binding to cells expressing CD63 or tumor-associated antigen, as determined by e.g. a FACS binding assay. Thus, reduced or weaker binding is observed with an increased EC₅₀, or half maximal effective concentration value.

In one embodiment, decreased binding can be defined as an increased EC₅₀ antibody concentration which enables binding to the half-maximal amount of target cells.

### Sequence Variants

The anti-CD63 antibodies and antigen-binding fragments thereof (including multispecific antigen-binding molecules and multidomain therapeutic proteins comprising anti-CD63 antibodies or antigen-binding fragments thereof) of the present invention may comprise one or more amino acid substitutions, insertions and/or deletions in the framework and/or CDR regions of the heavy and light chain variable domains as compared to the corresponding germline sequences from which the individual antigen-binding domains were derived. Such mutations can be readily ascertained by comparing the amino acid sequences disclosed herein to germline sequences available from, for example, public antibody sequence databases. The antigen-binding molecules of the present invention may comprise antigen-binding domains which are derived from any of the exemplary amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antigen-binding domain was originally derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antigen-binding domain was originally derived). Furthermore, the antigen-binding domains may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antigen-binding domains that contain one or more germline mutations can be easily tested for one or more desired property such as, improved binding specificity, increased binding affinity, improved or enhanced antagonistic or agonistic biological properties (as the case may be), reduced immunogenicity, etc. Bispecific antigen-binding molecules comprising one or more antigen-binding domains obtained in this general manner are encompassed within the present invention.

The present invention also includes antigen-binding molecules wherein one or both antigen-binding domains comprise variants of any of the HCVR, LCVR, and/or CDR amino acid sequences disclosed herein having one or more conservative substitutions. For example, the present invention includes antigen-binding molecules comprising an antigen-binding domain having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445, herein incorporated by reference. A “moderately conservative” replacement is any change having a nonnegative value in the PAM250 log-likelihood matrix.

The present invention also includes antigen-binding molecules comprising an antigen-binding domain with an HCVR, LCVR, and/or CDR amino acid sequence that is substantially identical to any of the HCVR, LCVR, and/or CDR amino acid sequences disclosed herein. The term “substantial identity” or “substantially identical,” when referring to an amino acid sequence means that two amino acid sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402, each herein incorporated by reference.

pH-Dependent Binding

The present invention includes anti-CD63 antibodies and antigen-binding fragments thereof (including multispecific antigen-binding molecules and multidomain therapeutic proteins comprising anti-CD63 antibodies or antigen-binding fragments thereof) with pH-dependent binding characteristics. For example, an anti-CD63 antibody of the present invention may exhibit reduced binding to CD63 at acidic pH as compared to neutral pH. Alternatively, anti-CD63 antibodies of the invention may exhibit enhanced binding to CD63 at acidic pH as compared to neutral pH. The expression “acidic pH” includes pH values less than about 6.2, e.g., about 6.0, 5.95, 5,9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. The expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding . . . at acidic pH as compared to neutral pH” is expressed in terms of a ratio of the K_{D} value of the antibody binding to its antigen at acidic pH to the K_{D} value of the antibody binding to its antigen at neutral pH (or vice versa). For example, an antibody or antigen-binding fragment thereof may be regarded as exhibiting “reduced binding to CD63 at acidic pH as compared to neutral pH” for purposes of the present invention if the antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment of the present invention can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0. 25.0, 30.0, 40.0, 50.0, 60.0, 70.0, 100.0 or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained.

### Antibodies Comprising Fc Variants

According to certain embodiments of the present invention, anti-CD63 antibodies and antigen-binding fragments thereof (including multispecific antigen-binding molecules and multidomain therapeutic proteins comprising anti-CD63 antibodies or antigen-binding fragments thereof) are provided comprising an Fc domain comprising one or more mutations which enhance or diminish antibody binding to the FcRn receptor, e.g., at acidic pH as compared to neutral pH. For example, the present invention includes antibodies comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P).

For example, the present invention includes anti-CD63 antibodies and antigen-binding fragments thereof (including multispecific antigen-binding molecules and multidomain therapeutic proteins comprising anti-CD63 antibodies or antigen-binding fragments thereof) comprising an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); and 433K and 434F (e.g., H433K and N434F). All possible combinations of the foregoing Fc domain mutations, and other mutations within the antibody variable domains disclosed herein, are contemplated within the scope of the present invention.

### Biological Characteristics of the Antibodies and Bispecific Antigen-Binding Molecules

The present invention includes antibodies and antigen-binding fragments thereof that bind human CD63 with high, medium or low affinity, depending on the therapeutic context and particular targeting properties that are desired. For example, in the context of a bispecific antigen-binding molecule, wherein one arm binds CD63 and another arm binds a target antigen (e.g., a tumor associated antigen), it may be desirable for the target antigen-binding arm to bind the target antigen with high affinity while the anti-CD63 arm binds CD63 with only moderate or low affinity. In this manner, preferential targeting of the antigen-binding molecule to cells expressing the target antigen may be achieved while avoiding general/untargeted CD63 binding and the consequent adverse side effects associated therewith.

The present invention includes antibodies, antigen-binding fragments, and bispecific antibodies thereof that bind human CD63 with weak (i.e. low) or even no detectable affinity. According to certain embodiments, the present invention includes antibodies and antigen-binding fragments of antibodies that bind human CD63 (e.g., at 37° C.) with a K_{D} of greater than about 100 nM as measured by surface plasmon resonance. In certain embodiments, the antibodies or antigen-binding fragments of the present invention bind CD63 with a K_{D} of greater than about greater than about 110 nM, at least 120 nM, greater than about 130 nM, greater than about 140 nM, greater than about 150 nM, at least 160 nM, greater than about 170 nM, greater than about 180 nM, greater than about 190 nM, greater than about 200 nM, greater than about 250 nM, greater than about 300 nM, greater than about 400 nM, greater than about 500 nM, greater than about 600 nM, greater than about 700 nM, greater than about 800 nM, greater than about 900 nM, or greater than about 1 μM, or with no detectable affinity, as measured by surface plasmon resonance (e.g., mAb-capture or antigen-capture format), or a substantially similar assay.

The present invention includes antibodies, antigen-binding fragments, and bispecific antibodies thereof that bind monkey (i.e. cynomolgus) CD63 with weak (i.e. low) or even no detectable affinity.

### Epitope Mapping and Related Technologies

The epitope on CD63 to which the anti-CD63 antibodies and antigen-binding fragments thereof (including multispecific antigen-binding molecules and multidomain therapeutic proteins comprising anti-CD63 antibodies or antigen-binding fragments thereof) of the present invention bind may consist of a single contiguous sequence of 3 or more (e.g., 3, 4, 5, 6, 7, 8, 9, 10, 11, 12, 13, 14, 15, 16, 17, 18, 19, 20 or more) amino acids of a CD63 protein. Alternatively, the epitope may consist of a plurality of non-contiguous amino acids (or amino acid sequences) of CD63. The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstances, an epitope may include moieties of saccharides, phosphoryl groups, or sulfonyl groups on the antigen.

Various techniques known to persons of ordinary skill in the art can be used to determine whether an antigen-binding domain of an antibody “interacts with one or more amino acids” within a polypeptide or protein. Exemplary techniques include, e.g., routine cross-blocking assay such as that described Antibodies, Harlow and Lane (Cold Spring Harbor Press, Cold Spring Harb., NY), alanine scanning mutational analysis, peptide blots analysis (Reineke, 2004, Methods Mol Biol 248:443-463), and peptide cleavage analysis. In addition, methods such as epitope excision, epitope extraction and chemical modification of antigens can be employed (Tomer, 2000, Protein Science 9:487-496). Another method that can be used to identify the amino acids within a polypeptide with which an antigen-binding domain of an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water to allow hydrogen-deuterium exchange to occur at all residues except for the residues protected by the antibody (which remain deuterium-labeled). After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267(2):252-259; Engen and Smith (2001) Anal. Chem. 73:256A-265A. X-ray crystallography of the antigen/antibody complex may also be used for epitope mapping purposes.

The present invention further includes anti-CD63 antibodies that bind to the same epitope as any of the specific exemplary antibodies described herein (e.g. antibodies comprising any of the amino acid sequences as set forth in Table 1 herein). Likewise, the present invention also includes anti-CD63 antibodies that compete for binding to CD63 with any of the specific exemplary antibodies described herein (e.g. antibodies comprising any of the amino acid sequences as set forth in Table 1 herein).

One can easily determine whether a particular antigen-binding molecule (e.g., antibody) or antigen-binding domain thereof binds to the same epitope as, or competes for binding with, a reference antigen-binding molecule of the present invention by using routine methods known in the art. For example, to determine if a test antibody binds to the same epitope on CD63 as a reference bispecific antigen-binding molecule of the present invention, the reference bispecific molecule is first allowed to bind to a CD63 protein. Next, the ability of a test antibody to bind to the CD63 molecule is assessed. If the test antibody is able to bind to CD63 following saturation binding with the reference bispecific antigen-binding molecule, it can be concluded that the test antibody binds to a different epitope of CD63 than the reference bispecific antigen-binding molecule. On the other hand, if the test antibody is not able to bind to the CD63 molecule following saturation binding with the reference bispecific antigen-binding molecule, then the test antibody may bind to the same epitope of CD63 as the epitope bound by the reference bispecific antigen-binding molecule of the invention. Additional routine experimentation (e.g., peptide mutation and binding analyses) can then be carried out to confirm whether the observed lack of binding of the test antibody is in fact due to binding to the same epitope as the reference bispecific antigen-binding molecule or if steric blocking (or another phenomenon) is responsible for the lack of observed binding. Experiments of this sort can be performed using ELISA, RIA, Biacore, flow cytometry or any other quantitative or qualitative antibody-binding assay available in the art. In accordance with certain embodiments of the present invention, two antigen-binding proteins bind to the same (or overlapping) epitope if, e.g., a 1-, 5-, 10-, 20- or 100-fold excess of one antigen-binding protein inhibits binding of the other by at least 50% but preferably 75%, 90% or even 99% as measured in a competitive binding assay (see, e.g., Junghans et al., Cancer Res. 1990:50:1495-1502). Alternatively, two antigen-binding proteins are deemed to bind to the same epitope if essentially all amino acid mutations in the antigen that reduce or eliminate binding of one antigen-binding protein reduce or eliminate binding of the other. Two antigen-binding proteins are deemed to have “overlapping epitopes” if only a subset of the amino acid mutations that reduce or eliminate binding of one antigen-binding protein reduce or eliminate binding of the other.

To determine if an antibody or antigen-binding domain thereof competes for binding with a reference antigen-binding molecule, the above-described binding methodology is performed in two orientations: In a first orientation, the reference antigen-binding molecule is allowed to bind to a CD63 protein under saturating conditions followed by assessment of binding of the test antibody to the CD63 molecule. In a second orientation, the test antibody is allowed to bind to a CD63 molecule under saturating conditions followed by assessment of binding of the reference antigen-binding molecule to the CD63 molecule. If, in both orientations, only the first (saturating) antigen-binding molecule is capable of binding to the CD63 molecule, then it is concluded that the test antibody and the reference antigen-binding molecule compete for binding to CD63. As will be appreciated by a person of ordinary skill in the art, an antibody that competes for binding with a reference antigen-binding molecule may not necessarily bind to the same epitope as the reference antibody, but may sterically block binding of the reference antibody by binding an overlapping or adjacent epitope.

### Preparation of Antigen-Binding Domains and Construction of Bispecific Molecules

Antigen-binding domains specific for particular antigens can be prepared by any antibody generating technology known in the art. Once obtained, two different antigen-binding domains, specific for two different antigens (e.g., CD63 and a target antigen), can be appropriately arranged relative to one another to produce a bispecific antigen-binding molecule of the present invention using routine methods. (A discussion of exemplary bispecific antibody formats that can be used to construct the bispecific antigen-binding molecules of the present invention is provided elsewhere herein). In certain embodiments, one or more of the individual components (e.g., heavy and light chains) of the multispecific antigen-binding molecules of the invention are derived from chimeric, humanized or fully human antibodies. Methods for making such antibodies are well known in the art. For example, one or more of the heavy and/or light chains of the bispecific antigen-binding molecules of the present invention can be prepared using VELOCIMMNE™ technology. Using VELOCIMMNE™ technology (or any other human antibody generating technology), high affinity chimeric antibodies to a particular antigen (e.g., CD63) are initially isolated having a human variable region and a mouse constant region. The antibodies are characterized and selected for desirable characteristics, including affinity, selectivity, epitope, etc. The mouse constant regions are replaced with a desired human constant region to generate fully human heavy and/or light chains that can be incorporated into the bispecific antigen-binding molecules of the present invention.

Genetically engineered animals may be used to make human bispecific antigen-binding molecules. For example, a genetically modified mouse can be used which is incapable of rearranging and expressing an endogenous mouse immunoglobulin light chain variable sequence, wherein the mouse expresses only one or two human light chain variable domains encoded by human immunoglobulin sequences operably linked to the mouse kappa constant gene at the endogenous mouse kappa locus. Such genetically modified mice can be used to isolate heavy chain and light chain variable regions to produce fully human bispecific antigen-binding molecules. As such, the fully human bispecific antigen-binding molecules comprise two different heavy chains that associate with the same light chain. (See, e.g., US 2011/0195454). Fully human refers to an antibody, or antigen-binding fragment or immunoglobulin domain thereof, comprising an amino acid sequence encoded by a DNA derived from a human sequence over the entire length of each polypeptide of the antibody or antigen-binding fragment or immunoglobulin domain thereof. In some instances, the fully human sequence is derived from a protein endogenous to a human. In other instances, the fully human protein or protein sequence comprises a chimeric sequence wherein each component sequence is derived from human sequence. While not being bound by any one theory, chimeric proteins or chimeric sequences are generally designed to minimize the creation of immunogenic epitopes in the junctions of component sequences, e.g. compared to any wild-type human immunoglobulin regions or domains.

Bispecific antigen-binding molecules may be constructed with one heavy chain having a modified Fc domain that abrogates its binding to Protein A, thus enabling a purification method that yields a heterodimeric protein. See, for example, U.S. Pat. No. 8,586,713. As such, the bispecific antigen-binding molecules comprise a first C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation/modification that reduces or abolishes Protein A binding such as an H95R modification (by IMGT exon numbering; H435R by EU numbering). The second C_{H}3 may further comprise a Y96F modification (by IMGT; Y436F by EU).

### Bioequivalents

The present invention encompasses antigen-binding molecules having amino acid sequences that vary from those of the exemplary molecules disclosed herein but that retain the ability to bind CD63. Such variant molecules may comprise one or more additions, deletions, or substitutions of amino acids when compared to parent sequence, but exhibit biological activity that is essentially equivalent to that of the described bispecific antigen-binding molecules.

The present invention includes antigen-binding molecules that are bioequivalent to any of the exemplary antigen-binding molecules set forth herein. Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antigen-binding proteins will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antigen-binding protein.

Bioequivalent variants of the exemplary bispecific antigen-binding molecules set forth herein may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antigen-binding proteins may include variants of the exemplary bispecific antigen-binding molecules set forth herein comprising amino acid changes which modify the glycosylation characteristics of the molecules, e.g., mutations which eliminate or remove glycosylation.

### Species Selectivity and Species Cross-Reactivity

According to certain embodiments of the invention, antigen-binding molecules are provided which bind to human CD63 but not to CD63 from other species. The present invention also includes antigen-binding molecules that bind to human CD63 and to CD63 from one or more non-human species.

According to certain exemplary embodiments of the invention, antigen-binding molecules are provided which bind to human CD63 may bind or not bind, as the case may be, to one or more of mouse, rat, guinea pig, hamster, gerbil, pig, cat, dog, rabbit, goat, sheep, cow, horse, camel, cynomolgus, marmoset, rhesus or chimpanzee CD63.

### Antibody-Drug Conjugates (ADCs)

The present invention provides antibody-drug conjugates (ADCs) comprising an anti-CD63 antibodies and antigen-binding fragments thereof (including multispecific antigen-binding molecules and multidomain therapeutic proteins comprising anti-CD63 antibodies or antigen-binding fragments thereof) thereof conjugated to a therapeutic moiety such as a cytotoxic agent, a chemotherapeutic drug, immunosuppressant or a radioisotope. Anti-CD63 antibodies, or antigen-binding fragment thereof, conjugated to a therapeutic moiety are also provided. In general terms, the ADCs comprise: A−[L−P]_{y}, in which A is an antigen-binding molecule, e.g. an anti-CD63 antibody, or a fragment thereof (e.g., a fragment comprising at least a HCDR3 selected from any of the HCDR3 amino acid sequences listed in Table 1), L is a linker, P is the payload or therapeutic moiety (e.g., cytotoxic agent), and y is an integer from 1 to 30.

In various embodiments, the ADC comprises an anti-CD63 antibody or antigen-binding fragment thereof (e.g., an anti-target (TAA)xanti-CD63 antibody) that comprises the CDRs of a HCVR or a LCVR having the amino acid sequences of the SEQ ID NOs (e.g., SEQ ID NOs: 2, 18, 34, 50, 66, 82, 98, 114, 130, 146, 162, 178, 194, 210, 226, 242, 258, 274, 290, 298, 306, 314, 322, 330, 10, 26, 42, 58, 74, 90, 106, 122, 138, 154, 170, 186, 202, 218, 234, 250, 266, and 282) set forth in Table 1, or specific HCVR/LCVR pairs (e.g., SEQ ID NOs: 2/10, 18/26, 34/42, 50/58, 66/74, 82/90, 98/106, 114/122, 130/138, 146/154, 162/170, 178/186, 194/202, 210/218, 226/234, 242/250, 258/266, 274/282, 290/282, 298/282, 306/282, 314/282, 322/282, and 330/282). In some cases, the anti-CD63 antibody or fragment comprises CDRs with the amino acid sequences of the SEQ ID NOs (e.g., SEQ ID NOs: 4-6-8-12-14-16, 20-22-24-26-28-30-32, 36-38-40-44-46-48, 52-54-56-60-62-64; 68-70-72-76-78-80 84-86-88-92-94-96; 100-102-104-108-110-112, 116-118-120-124-126-128; 132-134-136-140-142-144; 148-150-152-156-158-160; 164-166-168-172-174-176; 180-182-184-188-190-192; 196-198-200-204-206-208;212-214-216-220-222-224; 228-230-232-236-238-240; 244-246-248-252-254-256; 260-262-264-268-270-272; 276-278-280-284-286-288;292-294-296-284-286-288;300-302-304-284-286-288; 308-310-312-284-286-288; 316-318-320-284-286-288; 324-326-328-284-286-288, and 332-334-336-284-286-288) set forth in Table 1. In some cases, the anti-CD63 antibody or fragment comprises a HCVR and a LCVR having the amino acid sequences of the SEQ ID NOs (e.g., SEQ ID NOs: 2, 18, 34, 50, 66, 82, 98, 114, 130, 146, 162, 178, 194, 210, 226, 242, 258, 274, 290, 298, 306, 314, 322, 330, 10, 26, 42, 58, 74, 90, 106, 122, 138, 154, 170, 186, 202, 218, 234, 250, 266, and 282) set forth in Table 1, or specific amino acid sequence pairs (e.g., SEQ ID NOs: 2/10, 18/26, 34/42, 50/58, 66/74, 82/90, 98/106, 114/122, 130/138, 146/154, 162/170, 178/186, 194/202, 210/218, 226/234, 242/250, 258/266, 274/282, 290/282, 298/282, 306/282, 314/282, 322/282, and 330/282).

Cytotoxic agents include any agent that is detrimental to the growth, viability or propagation of cells, including, but not limited to, tubulin-interacting agents and DNA-damaging agents. Examples of suitable cytotoxic agents and chemotherapeutic agents that can be conjugated to anti-CD63 antibodies in accordance with this aspect of the disclosure include, e.g., 1-(2chloroethyl)-1,2-dimethanesulfonyl hydrazide, 1,8-dihydroxy-bicyclo[7.3.1]trideca-4,9-diene-2,6-diyne-13-one, 1-dehydrotestosterone, 5-fluorouracil, 6-mercaptopurine, 6-thioguanine, 9-amino camptothecin, actinomycin D, amanitins, aminopterin, anguidine, anthracycline, anthramycin (AMC), auristatins, bleomycin, busulfan, butyric acid, calicheamicins (e.g., calicheamicin γ1), camptothecin, carminomycins, carmustine, cemadotins, cisplatin, colchicin, combretastatins, cyclophosphamide, cytarabine, cytochalasin B, dactinomycin, daunorubicin, decarbazine, diacetoxypentyldoxorubicin, dibromomannitol, dihydroxy anthracin dione, disorazoles, dolastatin (e.g., dolastatin 10), doxorubicin, duocarmycin, echinomycins, eleutherobins, emetine, epothilones, esperamicin, estramustines, ethidium bromide, etoposide, fluorouracils, geldanamycins, gramicidin D, glucocorticoids, irinotecans, kinesin spindle protein (KSP) inhibitors, leptomycins, leurosines, lidocaine, lomustine (CCNU), maytansinoids, mechlorethamine, melphalan, mercatopurines, methopterins, methotrexate, mithramycin, mitomycin, mitoxantrone, N8-acetyl spermidine, podophyllotoxins, procaine, propranolol, pteridines, puromycin, pyrrolobenzodiazepines (PBDs), rhizoxins, streptozotocin, tallysomycins, taxol, tenoposide, tetracaine, thioepa chlorambucil, tomaymycins, topotecans, tubulysin, vinblastine, vincristine, vindesine, vinorelbines, and derivatives of any of the foregoing.

According to certain embodiments, the cytotoxic agent that is conjugated to an anti-CD63 antibody is a maytansinoid such as DM1 or DM4, a tomaymycin derivative, or a dolastatin derivative. According to certain embodiments, the cytotoxic agent that is conjugated to an anti-CD63 antibody is an auristatin such as MMAE, MMAF, or derivatives thereof. Other cytotoxic agents known in the art are contemplated within the scope of the present disclosure, including, e.g., protein toxins such ricin, C. difficile toxin, pseudomonas exotoxin, ricin, diphtheria toxin, botulinum toxin, bryodin, saporin, pokeweed toxins (i.e., phytolaccatoxin and phytolaccigenin), and others such as those set forth in Sapra et al., Pharmacol. & Therapeutics, 2013, 138:452-469.

In certain embodiments, the cytotoxic agent is a maytansinoid, e.g., derivative of maytansine. Suitable maytansinoids include DM1, DM4, or derivatives, stereoisomers, or isotopologues thereof. Suitable maytansinoids also include, but are not limited to, those disclosed in WO 2014/145090A1, WO 2015/031396A1, US 2016/0375147A1, and US 2017/0209591A1, incorporated herein by reference in their entireties.

In some embodiments, the maytansinoid has the following structure:

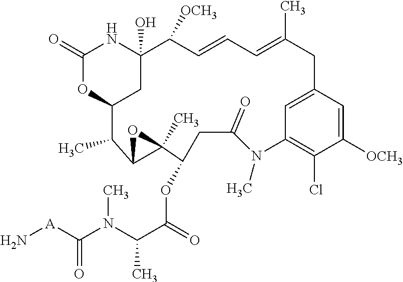

wherein A is an optionally substituted arylene or heteroarylene.

In some embodiments, the maytansinoid has the following structure:

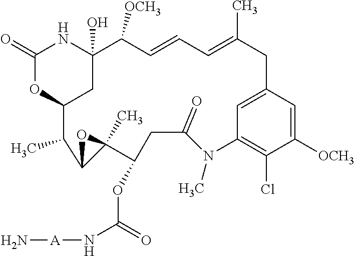

wherein A is an optionally substituted arylene or heteroarylene.

In some embodiments, the maytansinoid has the following structure:

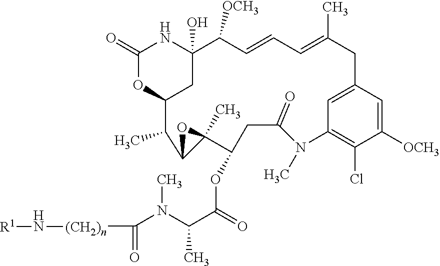

wherein n is an integer from 1-12 and R1 is alkyl.

In some embodiments, the maytansinoid is:

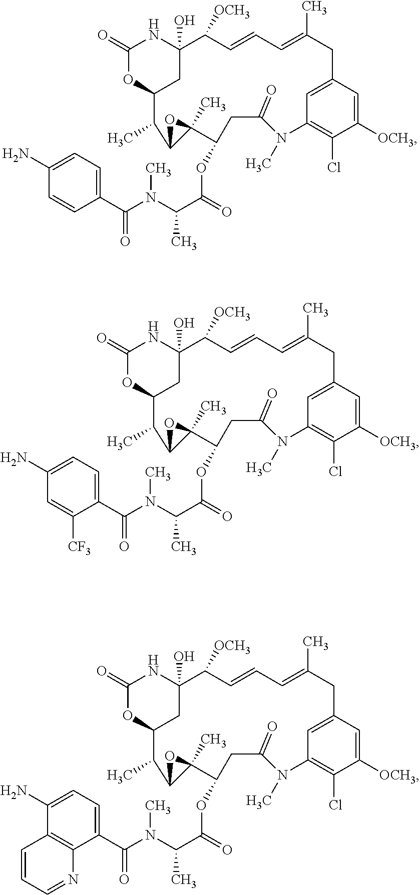 [SMILES: COc1cc2cc(c1Cl)N(C)C(=O)C[C@H](OC(=O)[C@H](C)N(C)C(=O)c1ccc(N)cc1)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)c1ccc(N)cc1C(F)(F)F)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)c1ccc(N)c3cccnc13)[C@]1(C)O[C@H]1[C@H](C)[C@@H]1C[C@@](O)(NC(=O)O1)[C@H](OC)/C=C/C=C(\C)C2]

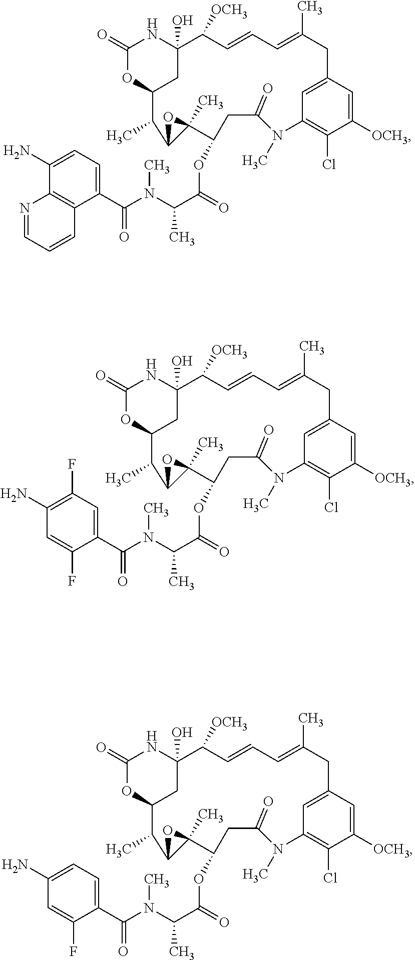 [SMILES: COc1cc2cc(c1Cl)N(C)C(=O)C[C@H](OC(=O)[C@H](C)N(C)C(=O)c1ccc(N)c3ncccc13)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)c1cc(F)c(N)cc1F)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)c1ccc(N)cc1F)[C@]1(C)O[C@H]1[C@H](C)[C@@H]1C[C@@](O)(NC(=O)O1)[C@H](OC)/C=C/C=C(\C)C2]

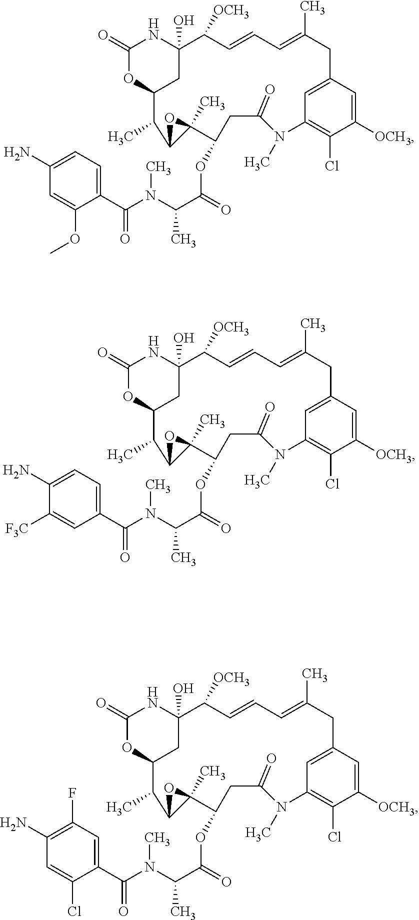 [SMILES: COc1cc(N)ccc1C(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C ; COc1cc2cc(c1Cl)N(C)C(=O)C[C@H](OC(=O)[C@H](C)N(C)C(=O)c1ccc(N)c(C(F)(F)F)c1)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)c1cc(F)c(N)cc1Cl)[C@]1(C)O[C@H]1[C@H](C)[C@@H]1C[C@@](O)(NC(=O)O1)[C@H](OC)/C=C/C=C(\C)C2]

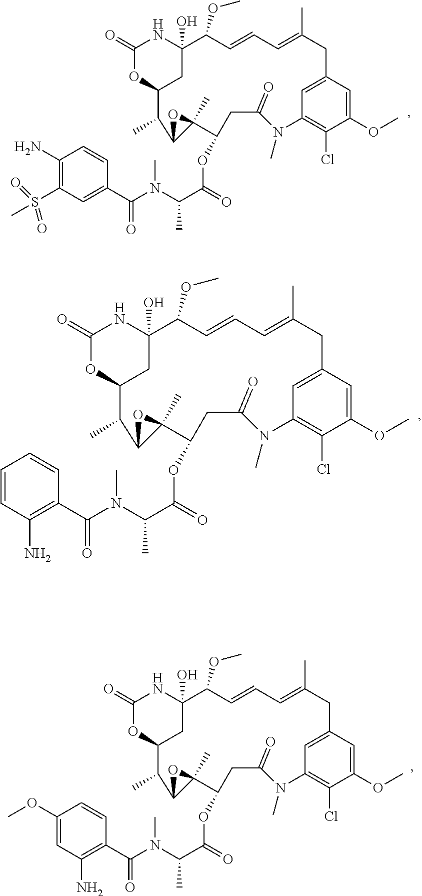

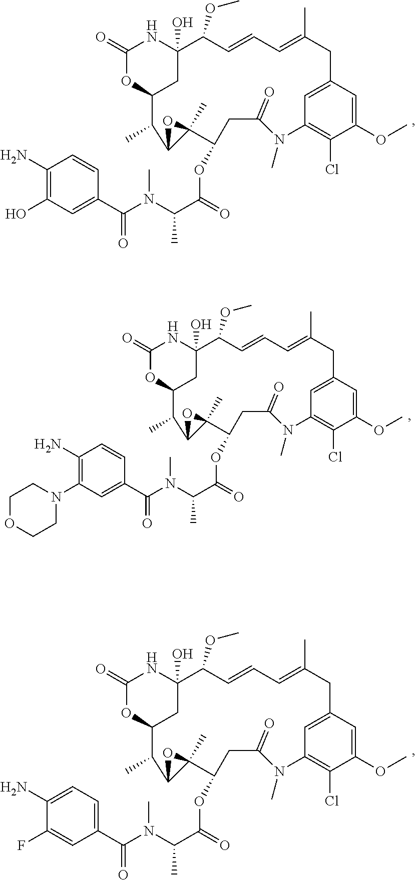 [SMILES: COc1cc2cc(c1Cl)N(C)C(=O)C[C@H](OC(=O)[C@H](C)N(C)C(=O)c1ccc(N)c(O)c1)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)c1ccc(N)c(N3CCOCC3)c1)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)c1ccc(N)c(F)c1)[C@]1(C)O[C@H]1[C@H](C)[C@@H]1C[C@@](O)(NC(=O)O1)[C@H](OC)/C=C/C=C(\C)C2]

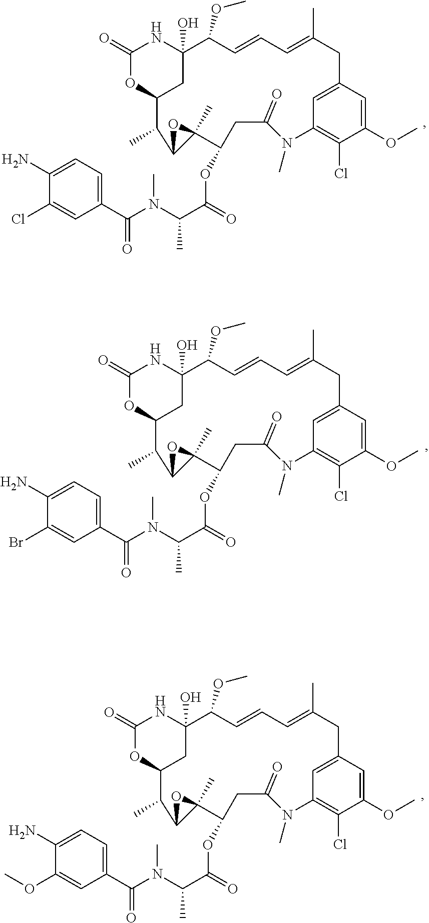

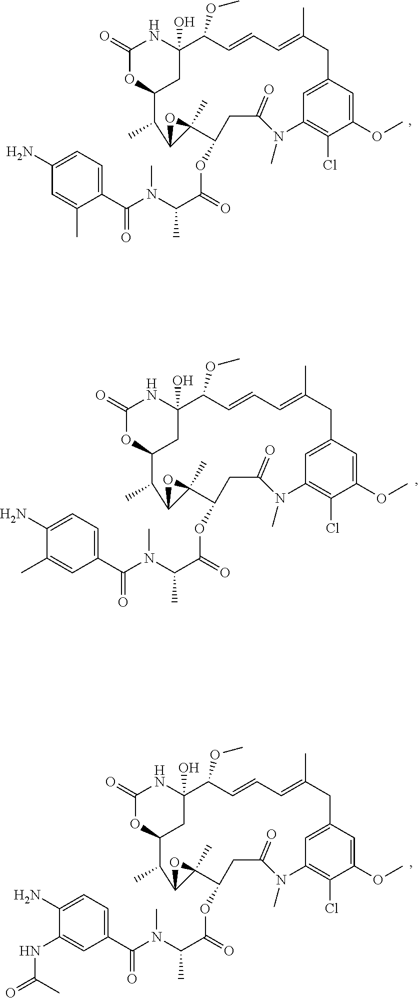 [SMILES: COc1cc2cc(c1Cl)N(C)C(=O)C[C@H](OC(=O)[C@H](C)N(C)C(=O)c1ccc(N)cc1C)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)c1ccc(N)c(C)c1)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)c1ccc(N)c(NC(C)=O)c1)[C@]1(C)O[C@H]1[C@H](C)[C@@H]1C[C@@](O)(NC(=O)O1)[C@H](OC)/C=C/C=C(\C)C2]

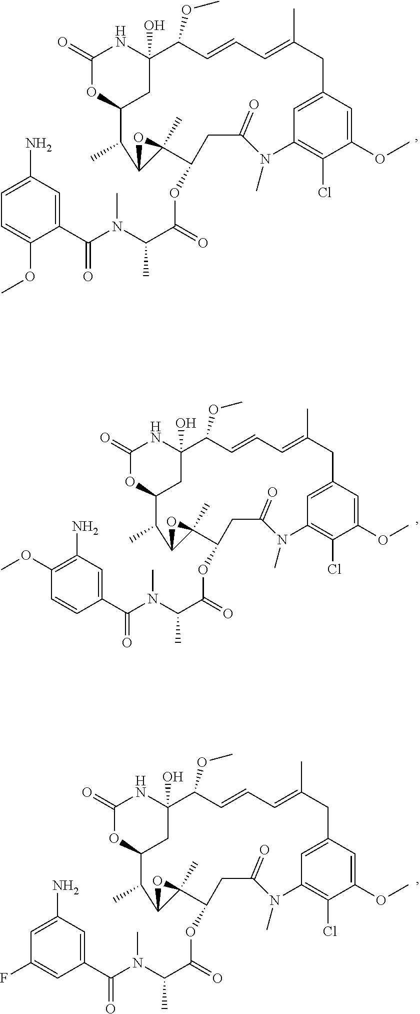

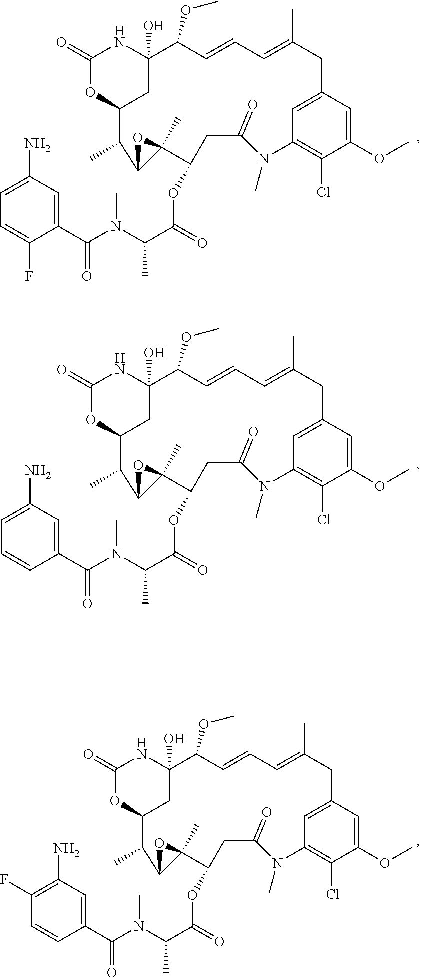 [SMILES: COc1cc2cc(c1Cl)N(C)C(=O)C[C@H](OC(=O)[C@H](C)N(C)C(=O)c1cc(N)ccc1F)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)c1cccc(N)c1)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)c1ccc(F)c(N)c1)[C@]1(C)O[C@H]1[C@H](C)[C@@H]1C[C@@](O)(NC(=O)O1)[C@H](OC)/C=C/C=C(\C)C2]

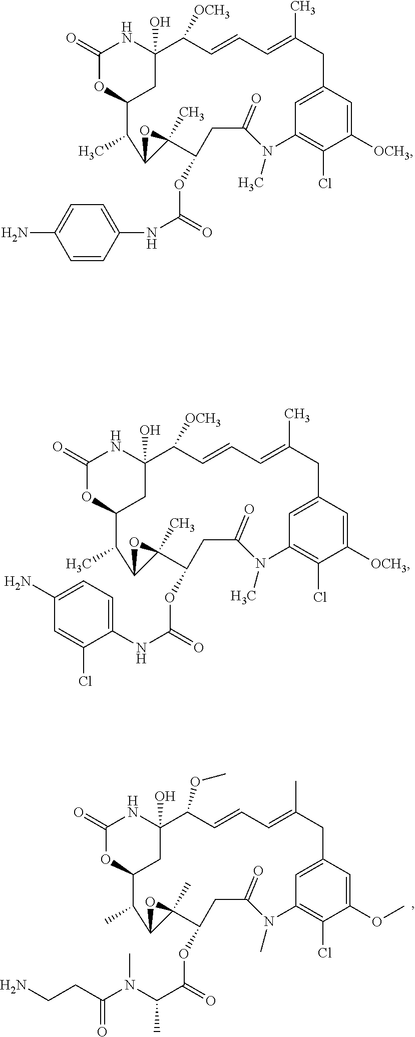 [SMILES: COc1cc2cc(c1Cl)N(C)C(=O)C[C@H](OC(=O)Nc1ccc(N)cc1)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)Nc1ccc(N)cc1Cl)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)CCN)[C@]1(C)O[C@H]1[C@H](C)[C@@H]1C[C@@](O)(NC(=O)O1)[C@H](OC)/C=C/C=C(\C)C2]

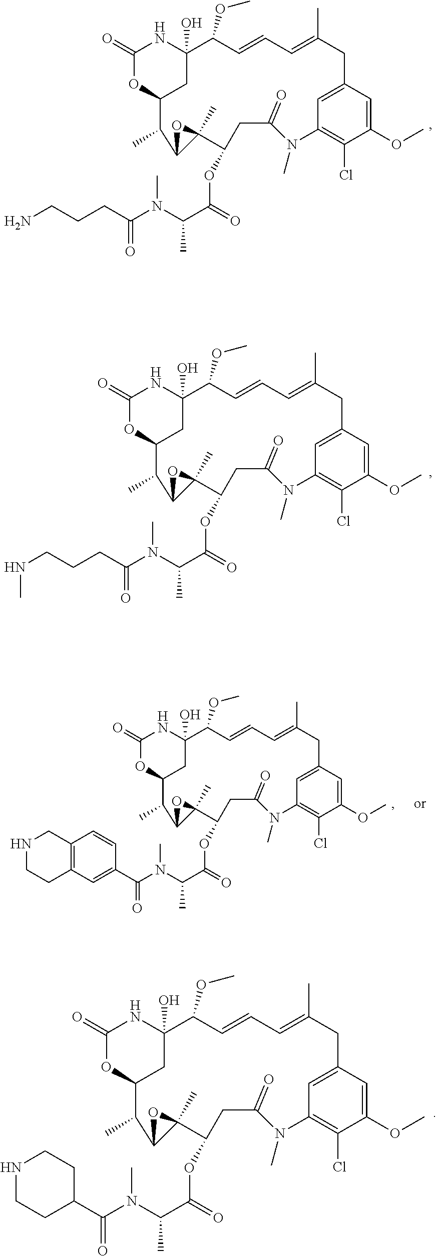

In some embodiments, the maytansinoid is:

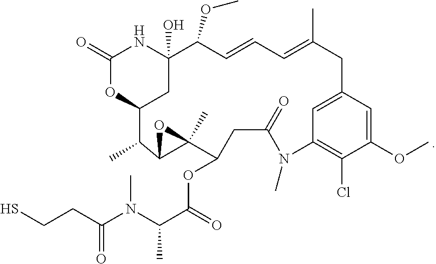 [SMILES: COc1cc2cc(c1Cl)N(C)C(=O)CC(OC(=O)[C@H](C)N(C)C(=O)CCS)[C@]1(C)O[C@H]1[C@H](C)[C@@H]1C[C@@](O)(NC(=O)O1)[C@H](OC)/C=C/C=C(\C)C2]

In some embodiments, the maytansinoid is:

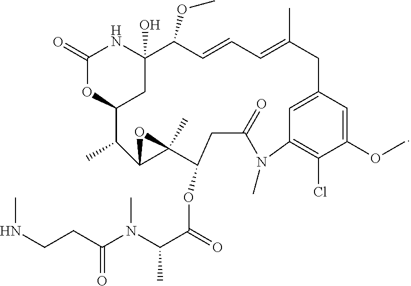 [SMILES: CNCCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

Also provided herein are antibody-radionuclide conjugates (ARCs) comprising anti-CD63 antibodies conjugated to one or more radionuclides. Exemplary radionuclides that can be used in the context of this aspect of the disclosure include, but are not limited to, e.g., ²²Ac, ²¹²Bi, ²¹³Bi, ¹³¹I, ¹⁸⁶Re, ²²⁷Th, ²²²Rn, ²²³Ra, ²²⁴Ra, and ⁹⁰Y.

In certain embodiments provided herein, ADCs are provided comprising, e.g., an anti-TAA x anti-CD63 bispecific antigen-binding protein conjugated to a cytotoxic agent (e.g., any of the cytotoxic agents disclosed above) via a linker molecule. Linkers are any group or moiety that links, connects, or bonds the antibody or antigen-binding proteins described herein with a therapeutic moiety, e.g. cytotoxic agent. Suitable linkers may be found, for example, in Antibody-Drug Conjugates and Immunotoxins; Phillips, G. L., Ed.; Springer Verlag: New York, 2013; Antibody-Drug Conjugates; Ducry, L., Ed.; Humana Press, 2013; Antibody-Drug Conjugates; Wang, J., Shen, W.-C., and Zaro, J. L., Eds.; Springer International Publishing, 2015, the contents of each incorporated herein in their entirety by reference. Generally, suitable binding agent linkers for the antibody conjugates described herein are those that are sufficiently stable to exploit the circulating half-life of the antibody and, at the same time, capable of releasing its payload after antigen-mediated internalization of the conjugate. Linkers can be cleavable or non-cleavable. Cleavable linkers include linkers that are cleaved by intracellular metabolism following internalization, e.g., cleavage via hydrolysis, reduction, or enzymatic reaction. Non-cleavable linkers include linkers that release an attached payload via lysosomal degradation of the antibody following internalization. Suitable linkers include, but are not limited to, acid-labile linkers, hydrolysis-labile linkers, enzymatically cleavable linkers, reduction labile linkers, self-immolative linkers, and non-cleavable linkers. Suitable linkers also include, but are not limited to, those that are or comprise peptides, glucuronides, succinimide-thioethers, polyethylene glycol (PEG) units, hydrazones, mal-caproyl units, dipeptide units, valine-citruline units, and para-aminobenzyl (PAB) units.

Any linker molecule or linker technology known in the art can be used to create or construct an ADC of the present disclosure. In certain embodiments, the linker is a cleavable linker. According to other embodiments, the linker is a non-cleavable linker. Exemplary linkers that can be used in the context of the present disclosure include, linkers that comprise or consist of e.g., MC (6-maleimidocaproyl), MP (maleimidopropanoyl), val-cit (valine-citrulline), val-ala (valine-alanine), val-gly (valine-glycine), dipeptide site in protease-cleavable linker, ala-phe (alanine-phenylalanine), dipeptide site in protease-cleavable linker, PAB (p-aminobenzyloxycarbonyl), SPP (N-Succinimidyl 4-(2-pyridylthio) pentanoate), SMCC (N-Succinimidyl 4-(N-maleimidomethyl)cyclohexane-1 carboxylate), SIAB (N-Succinimidyl (4-iodo-acetyl)aminobenzoate), and variants and combinations thereof. Additional examples of linkers that can be used in the context of the present disclosure are provided, e.g., in U.S. Pat. No. 7,754,681 and in Ducry, Bioconjugate Chem., 2010, 21:5-13, and the references cited therein, the contents of which are incorporated by reference herein in their entireties.

In certain embodiments, the linkers are stable in physiological conditions. In certain embodiments, the linkers are cleavable, for instance, able to release at least the payload portion in the presence of an enzyme or at a particular pH range or value. In some embodiments, a linker comprises an enzyme-cleavable moiety. Illustrative enzyme-cleavable moieties include, but are not limited to, peptide bonds, ester linkages, hydrazones, and disulfide linkages. In some embodiments, the linker comprises a cathepsin-cleavable linker.

In some embodiments, the linker comprises a non-cleavable moiety.

Suitable linkers also include, but are not limited to, those that are chemically bonded to two cysteine residues of a single binding agent, e.g., antibody. Such linkers can serve to mimic the antibody's disulfide bonds that are disrupted as a result of the conjugation process.

In some embodiments, the linker comprises one or more amino acids. Suitable amino acids include natural, non-natural, standard, non-standard, proteinogenic, non-proteinogenic, and L- or D-α-amino acids. In some embodiments, the linker comprises alanine, valine, glycine, leucine, isoleucine, methionine, tryptophan, phenylalanine, proline, serine, threonine, cysteine, tyrosine, asparagine, glutamine, aspartic acid, glutamic acid, lysine, arginine, histidine, or citrulline, a derivative thereof, or combination thereof. In certain embodiments, one or more side chains of the amino acids is linked to a side chain group, described below. In some embodiments, the linker comprises valine and citrulline. In some embodiments, the linker comprises lysine, valine, and citrulline. In some embodiments, the linker comprises lysine, valine, and alanine. In some embodiments, the linker comprises valine and alanine.

In some embodiments, the linker comprises a self-immolative group. The self-immolative group can be any such group known to those of skill. In particular embodiments, the self-immolative group is p-aminobenzyl (PAB), or a derivative thereof. Useful derivatives include p-aminobenzyloxycarbonyl (PABC). Those of skill will recognize that a self-immolative group is capable of carrying out a chemical reaction which releases the remaining atoms of a linker from a payload.

In some embodiments, the linker is:

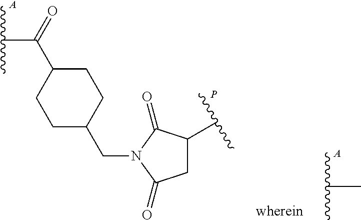

is a bond to the antibody or antigen-binding protein (e.g., via lysine residue) and

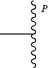

is a bond to the cytotoxic agent (e.g., DM1). In some embodiments, the linker is:

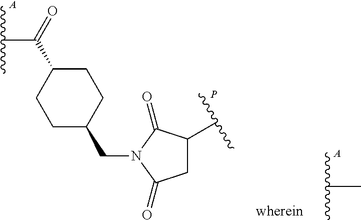

is a bond to the antibody or antigen-binding protein (e.g., via lysine residue) and

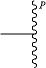

is a bond to the cytotoxic agent (e.g., DM1). In certain embodiments, the linker is:

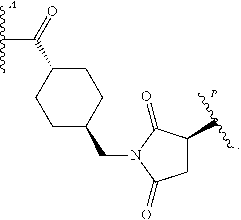

In certain embodiments, the linker is:

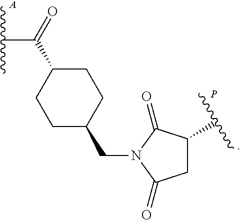

In some embodiments, the linker is derived from maleimidylmethyl-4-trans-cyclohexanecarboxysuccinate:

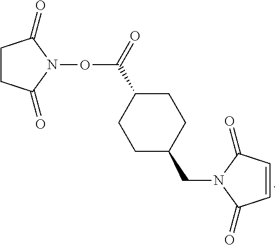 [SMILES: O=C1C=CC(=O)N1C[C@H]1CC[C@H](C(=O)ON2C(=O)CCC2=O)CC1]

In some embodiments, the linker is:

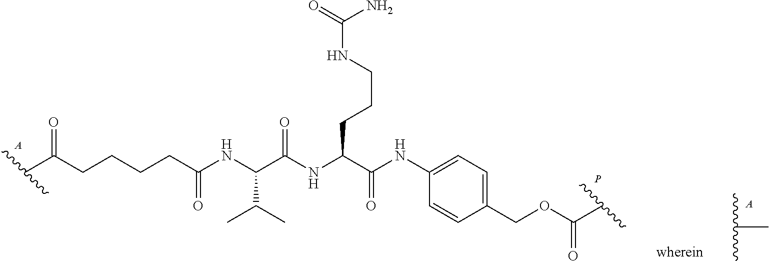

is a bond to the antibody or antigen-binding protein (e.g., via lysine residue) and

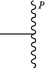

is a bond to the cytotoxic agent (e.g., a compound having the following formula:

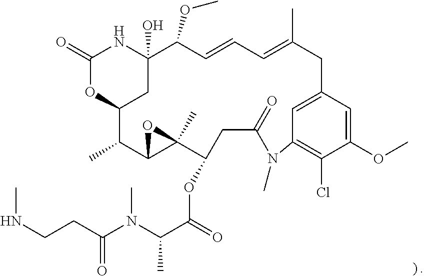 [SMILES: CNCCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

The present disclosure comprises ADCs in which a linker connects an anti-TAA x anti-CD63 bispecific antigen-binding protein to a drug or cytotoxin through an attachment at a particular amino acid within the antibody or antigen-binding molecule. Exemplary amino acid attachments that can be used in the context of this aspect, e.g., lysine (see, e.g., U.S. Pat. No. 5,208,020; US 2010/0129314; Hollander et al., Bioconjugate Chem., 2008, 19:358-361; WO 2005/089808; U.S. Pat. No. 5,714,586; and US 2013/0101546), cysteine (see, e.g., US 2007/0258987; WO 2013/055993; WO 2013/055990; WO 2013/053873; WO 2013/053872; WO 2011/130598; US 2013/0101546; and U.S. Pat. No. 7,750,116), selenocysteine (see, e.g., WO 2008/122039; and Hofer et al., Proc. Natl. Acad. Sci., USA, 2008, 105:12451-12456), formyl glycine (see, e.g., Carrico et al., Nat. Chem. Biol., 2007, 3:321-322; Agarwal et al., Proc. Natl. Acad. Sci., USA, 2013, 110:46-51, and Rabuka et al., Nat. Protocols, 2012, 10:1052-1067), non-natural amino acids (see, e.g., WO 2013/068874, and WO 2012/166559), and acidic amino acids (see, e.g., WO 2012/05982). Linkers can also be conjugated to an antigen-binding protein via attachment to carbohydrates (see, e.g., US 2008/0305497, WO 2014/065661, and Ryan et al., Food & Agriculture Immunol., 2001, 13:127-130) and disulfide linkers (see, e.g., WO 2013/085925, WO 2010/010324, WO 2011/018611, and Shaunak et al., Nat. Chem. Biol., 2006, 2:312-313). Site specific conjugation techniques can also be employed to direct conjugation to particular residues of the antibody or antigen binding protein (see, e.g., Schumacher et al. J Clin Immunol (2016) 36(Suppl 1): 100). Site specific conjugation techniques, include, but are not limited to glutamine conjugation via transglutaminase (see e.g., Schibli, Angew Chemie Inter Ed. 2010, 49, 9995).

According to certain embodiments, the present disclosure provides ADCs, wherein an anti-TAA x anti-CD63 bispecific antigen-binding protein as described herein is conjugated to a linker-drug composition as set forth in International Patent Publication WO2014/145090, (e.g., compound “7,” also referred to herein as “M0026” and depicted below), the disclosure of which is hereby incorporated by reference herein in its entirety:

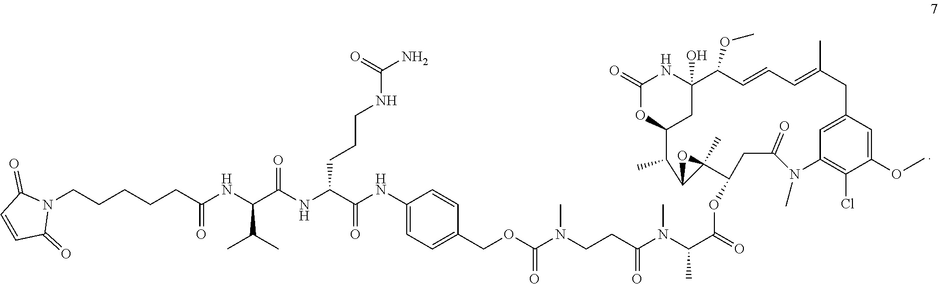 [SMILES: COc1cc2cc(c1Cl)N(C)C(=O)C[C@H](OC(=O)[C@H](C)N(C)C(=O)CCN(C)C(=O)OCc1ccc(NC(=O)[C@@H](CCCNC(N)=O)NC(=O)[C@H](NC(=O)CCCCCN3C(=O)C=CC3=O)C(C)C)cc1)[C@]1(C)O[C@H]1[C@H](C)[C@@H]1C[C@@](O)(NC(=O)O1)[C@H](OC)/C=C/C=C(\C)C2]

Provided herein are also antibody-drug conjugates comprising the anti-TAA x anti-CD63 bispecific antigen-binding proteins, where said anti-TAA x anti-CD63 bispecific antigen-binding protein is conjugated to a cytotoxic agent. In certain embodiments, the cytotoxic agent is a maytansinoid. In certain embodiments, the maytansinoid is a compound having the following formula:

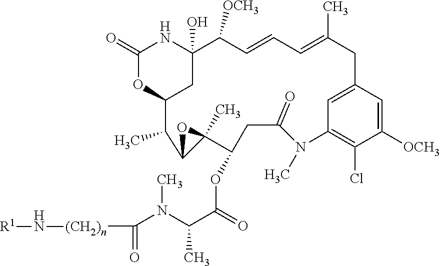

wherein n is an integer from 1-12 and R¹ is alkyl. In certain embodiments, the maytansinoid is

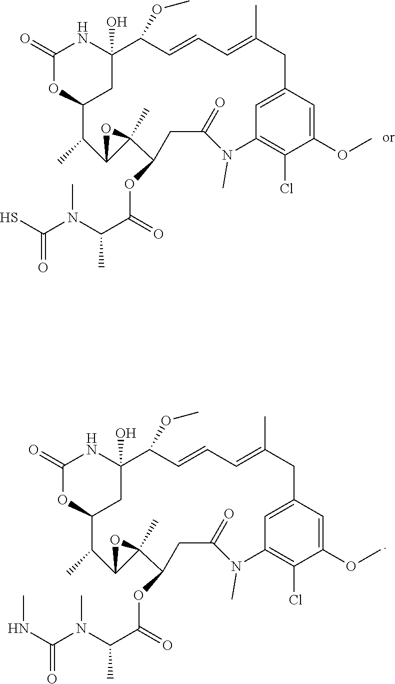 [SMILES: COc1cc2cc(c1Cl)N(C)C(=O)C[C@@H](OC(=O)[C@H](C)N(C)C(=O)S)[C@]1(C)O[C@H]1[C@H](C)[C@@H]1C[C@@](O)(NC(=O)O1)[C@H](OC)/C=C/C=C(\C)C2 ; CNC(=O)N(C)[C@@H](C)C(=O)O[C@@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

In certain embodiments, the cytotoxic agent is a maytansinoid, and the maytansinoid is covalently attached to the antibody via non-cleavable linker. In certain embodiments, the cytotoxic agent is a maytansinoid, and the maytansinoid is covalently attached to the antibody via cleavable linker.

In one embodiment, the antibody is conjugated to:

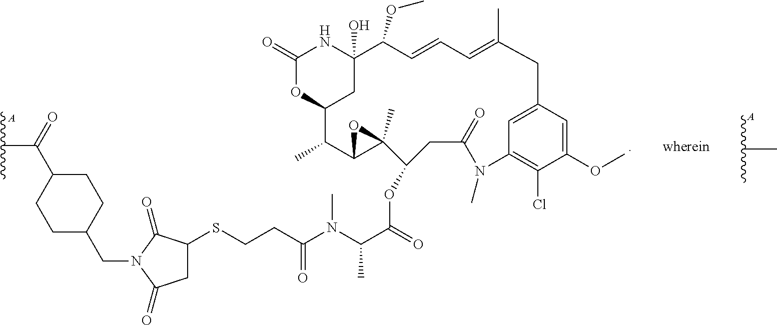

is a bond to the antibody.

In one embodiment, the antibody is conjugated to:

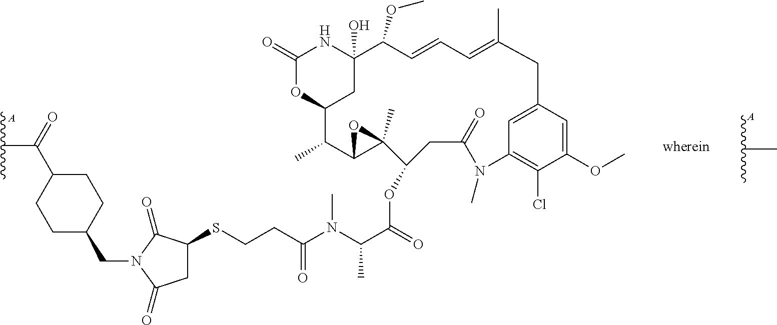

is a bond to the antibody.

In one embodiment, the antibody is conjugated to:

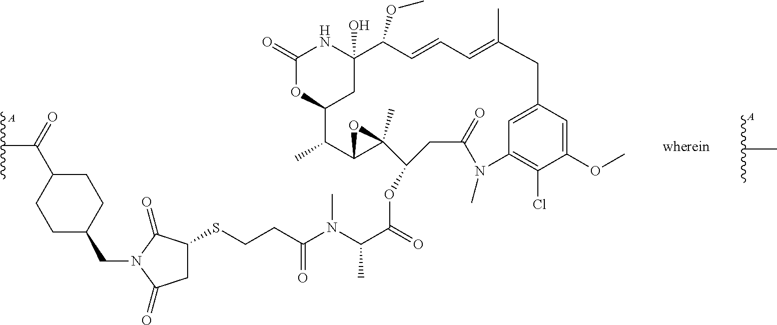

is a bond to the antibody.

In one embodiment, the antibody is conjugated to:

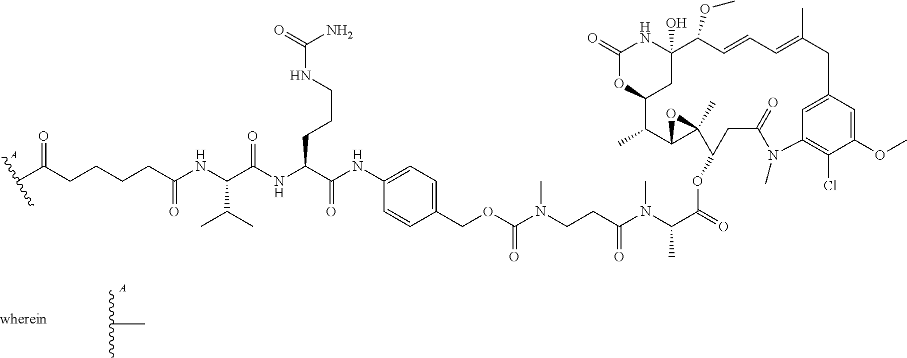

is a bond to the antibody.

In some embodiments, the conjugates have the following structure:

Ab-[L-Pay]ₙ

wherein:
Ab is an anti-TAA x anti-CD63 bispecific antigen-binding protein as described herein;
L is a linker;
Pay is a cytotoxic agent; and
n is an integer from 1-10.

In some embodiments, Ab is any of the antibodies or antigen-binding proteins described in Table 1.

In some embodiments, Payload is a maytansinoid.

In some embodiments, Pay is:

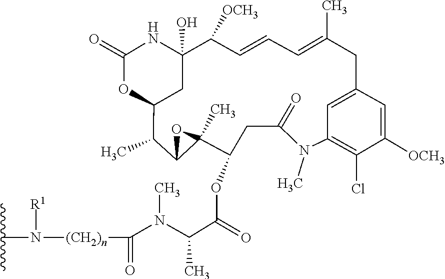

wherein R¹ is alkyl.

In some embodiments, Pay is:

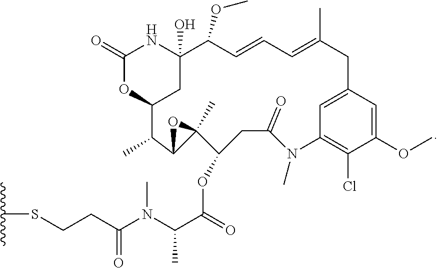 [SMILES: *SCCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

In some embodiments, Pay is:

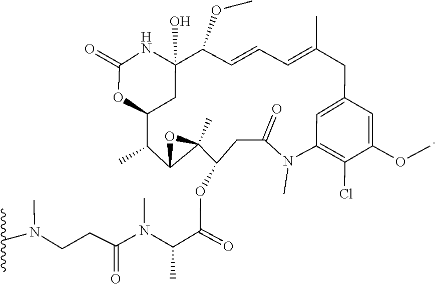 [SMILES: *N(C)CCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

In some embodiments, n is an integer from 2 to 5.

In some embodiments, -L-Pay is:

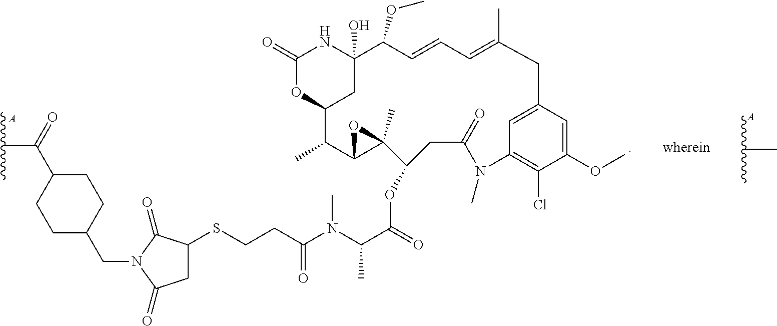

is a bond to the antibody.

In some embodiments, -L-Pay is:

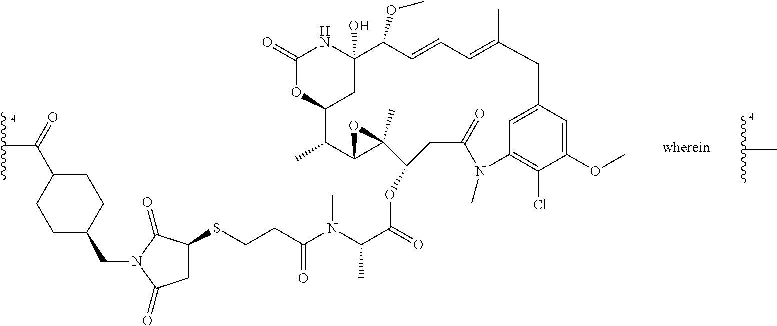

is a bond to the antibody.

In some embodiments, -L-Pay is

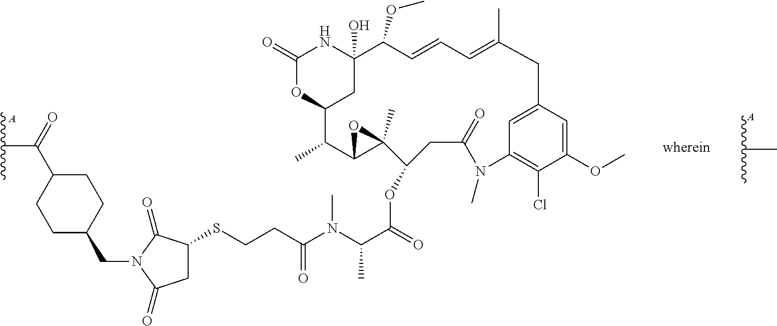

is a bond to the antibody.

In some embodiments, -L-Pay is:

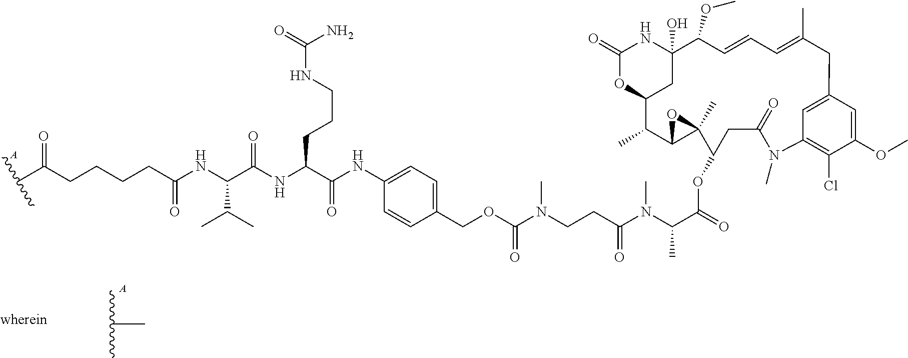

is a bond to the antibody.

In some embodiments, -L-Pay is:

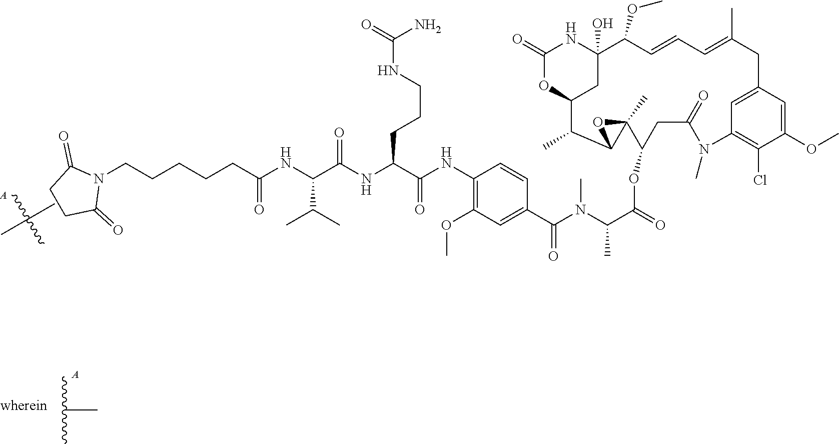

is a bond to the antibody.

The antibody drug conjugates described herein can be prepared using conjugation conditions known to those of ordinary skill in the art, (see, e.g., Doronina et al. Nature Biotechnology 2003, 21, 7, 778, which is incorporated herein by reference in its entirety). In some embodiments an anti-TAA x anti-CD63 bispecific antigen-binding protein drug conjugate is prepared by contacting an anti-TAA x anti-CD63 bispecific antigen-binding protein described herein with a compound comprising the desired linker and cytotoxic agent, wherein said linker possesses a moiety that is reactive with the antibody or antigen-binding protein, e.g., at the desired residue of the antibody or antigen-binding protein.

In some embodiments, provided herein are processes for preparing an antibody-drug conjugate comprising contacting an anti-TAA x anti-CD63 bispecific antigen-binding protein described herein with a compound having the following formula A¹:

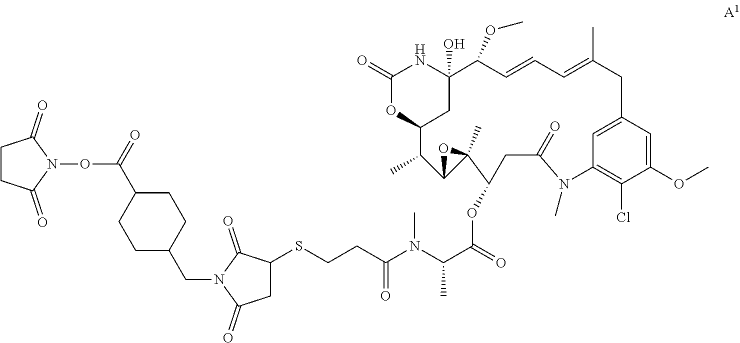 [SMILES: COc1cc2cc(c1Cl)N(C)C(=O)C[C@H](OC(=O)[C@H](C)N(C)C(=O)CCSC1CC(=O)N(CC3CCC(C(=O)ON4C(=O)CCC4=O)CC3)C1=O)[C@]1(C)O[C@H]1[C@H](C)[C@@H]1C[C@@](O)(NC(=O)O1)[C@H](OC)/C=C/C=C(\C)C2]

and aqueous diluent.

In some embodiments, the compound of formula A¹ is present in stoichiometric excess. In some embodiments, the compound of formula A¹ is present in 5-6 fold stoichiometric excess. In some embodiments, the aqueous diluent comprises HEPES. In some embodiments, the aqueous diluent comprises DMA.

In some embodiments, the compound of formula A¹ is a compound of formula A² or A³:

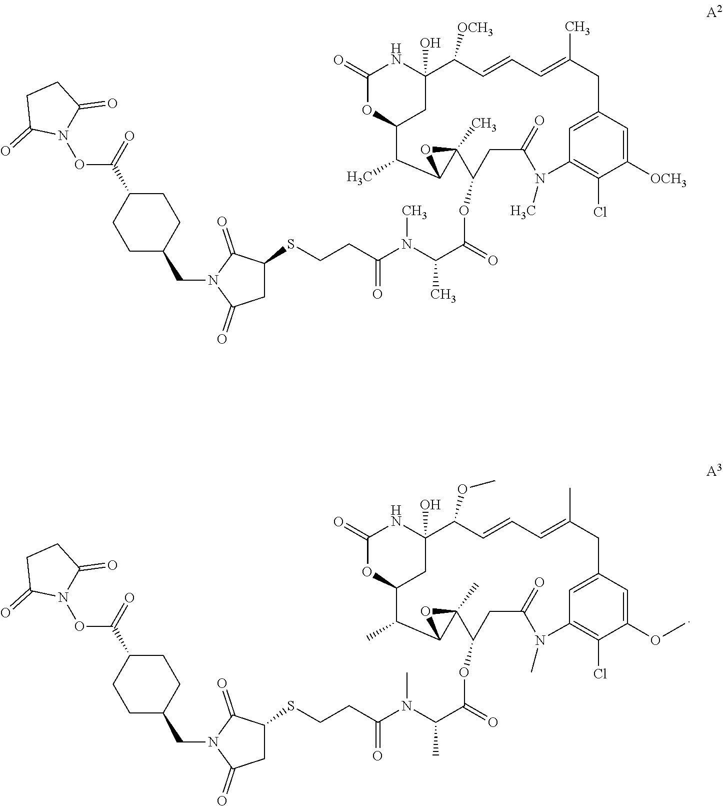 [SMILES: COc1cc2cc(c1Cl)N(C)C(=O)C[C@H](OC(=O)[C@H](C)N(C)C(=O)CCS[C@H]1CC(=O)N(C[C@H]3CC[C@H](C(=O)ON4C(=O)CCC4=O)CC3)C1=O)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)CCS[C@@H]1CC(=O)N(C[C@H]3CC[C@H](C(=O)ON4C(=O)CCC4=O)CC3)C1=O)[C@]1(C)O[C@H]1[C@H](C)[C@@H]1C[C@@](O)(NC(=O)O1)[C@H](OC)/C=C/C=C(\C)C2]

In some embodiments, the compound of formula A² is A³ stereomerically pure. In some embodiments, the compound of formula A¹ comprises a compound of formula A¹ or A², wherein the compound of A¹ or A² is present in a diastereomeric excess of more than 50%. In certain embodiments, the diastereomeric excess is more than 70%. In certain embodiments, the diastereomeric excess is more than 90%. In certain embodiments, the diastereomeric excess is more than 95%. Structures A¹, A² and A³ individually or collectively are known as SMCC-DM1.

The term “diastereomeric excess” refers to the difference between the mole fraction of the desired single diastereomer as compared to the remaining diastereomers in a composition. Diastereomeric excess is calculated as follows: (amount of single diastereomer)−(amount of other diastereomers)/1. For example, a composition that contains 90% of 1 and 10% of 2, 3, 4, or a mixture thereof has a diastereomeric excess of 80% [(90-10)/1]. A composition that contains 95% of 1 and 5% of 2, 3, 4, or a mixture thereof has a diastereomeric excess of 90% [(95-5)/1]. A composition that contains 99% of 1 and 1% of 2, 3, 4, or a mixture thereof has a diastereomeric excess of 98% [(99-1)/1]. The diastereomeric excess can similarly be calculated for any one of 1, 2, 3, or 4.

In some embodiments, the compound of formula A¹ is prepared by contacting a compound of formula (a):

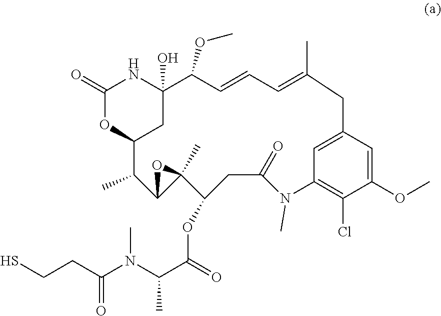 [SMILES: COc1cc2cc(c1Cl)N(C)C(=O)C[C@H](OC(=O)[C@H](C)N(C)C(=O)CCS)[C@]1(C)O[C@H]1[C@H](C)[C@@H]1C[C@@](O)(NC(=O)O1)[C@H](OC)/C=C/C=C(\C)C2]

with a compound of formula (b)

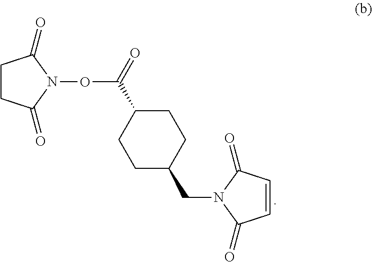 [SMILES: O=C1C=CC(=O)N1C[C@H]1CC[C@H](C(=O)ON2C(=O)CCC2=O)CC1]

in the presence of silica gel and diluent. In some embodiments, the diluent comprises an organic solvent and water.

Provided herein is also the product prepared by the process of:
(i) contacting a compound of formula (a):

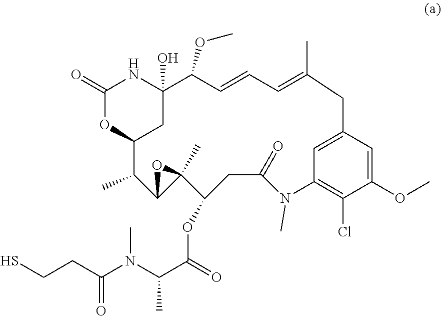 [SMILES: COc1cc2cc(c1Cl)N(C)C(=O)C[C@H](OC(=O)[C@H](C)N(C)C(=O)CCS)[C@]1(C)O[C@H]1[C@H](C)[C@@H]1C[C@@](O)(NC(=O)O1)[C@H](OC)/C=C/C=C(\C)C2]

with a compound of formula (b):

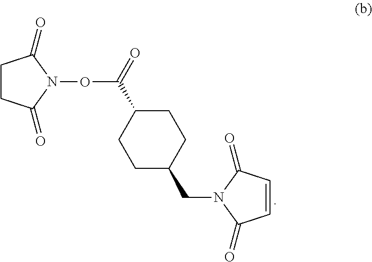 [SMILES: O=C1C=CC(=O)N1C[C@H]1CC[C@H](C(=O)ON2C(=O)CCC2=O)CC1]

in the presence of silica gel and diluent to synthesize an intermediate; and
(ii) contacting an anti-TAA x anti-CD63 bispecific antigen-binding protein with the intermediate and aqueous diluent.

In some embodiments, provided herein are processes for preparing an antibody-drug conjugate comprising contacting an anti-TAA x anti-CD63 bispecific antigen-binding protein described herein with a compound having the following formula B:

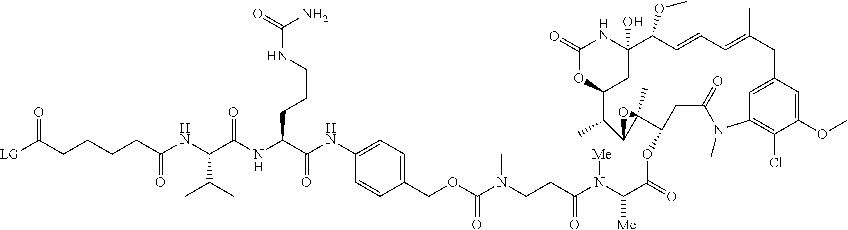

wherein LG is a leaving group, and aqueous diluent.

In some embodiments, the compound of formula B is present in stoichiometric excess. In some embodiments, the compound of formula B is present in 5-6 fold stoichiometric excess. In some embodiments, the aqueous diluent comprises HEPES. In some embodiments, the aqueous diluent comprises DMA. In some embodiments, the —C(O)-LG is an ester, e.g., NHS or pentafluorophenyl ester.

In some embodiments, the compound of formula B is a compound of formula B1 known as Compound I:

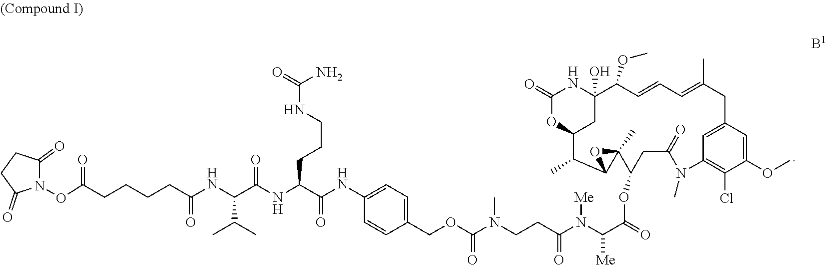 [SMILES: COc1cc2cc(c1Cl)N(C)C(=O)C[C@H](OC(=O)[C@H](C)N(C)C(=O)CCN(C)C(=O)OCc1ccc(NC(=O)[C@H](CCCNC(N)=O)NC(=O)[C@@H](NC(=O)CCCCC(=O)ON3C(=O)CCC3=O)C(C)C)cc1)[C@]1(C)O[C@H]1[C@H](C)[C@@H]1C[C@@](O)(NC(=O)O1)[C@H](OC)/C=C/C=C(\C)C2]

In some embodiments, the compound of formula C is known as Compound II:

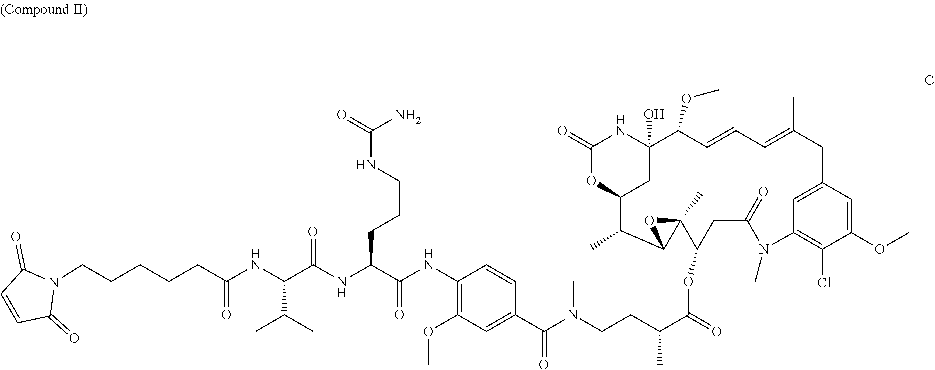 [SMILES: COc1cc(C(=O)N(C)CC[C@@H](C)C(=O)O[C@H]2CC(=O)N(C)c3cc(cc(OC)c3Cl)C/C(C)=C/C=C/[C@@H](OC)[C@@]3(O)C[C@H](OC(=O)N3)[C@@H](C)[C@@H]3O[C@@]23C)ccc1NC(=O)[C@H](CCCNC(N)=O)NC(=O)[C@@H](NC(=O)CCCCCN1C(=O)C=CC1=O)C(C)C]

Drug-to-antibody ratio (DAR) is the average number of drugs conjugated to the antibody or antigen-binding fragment, which has an important effect on the efficacy, potency and pharmacokinetics of the ADC. In various embodiments, the DAR is from 1, 2, 3, 4, 5, 6, 7, or 8 drug molecules per antibody. In some embodiments, the DAR is from 1 to 4. In certain embodiments, the DAR is from 2 to 4. In some cases, the DAR is from 2 to 3. In certain cases, the DAR is from 3 to 4. In some embodiments, the DAR is from 1 to 10, 1 to 20 or 1 to 30 (i.e., from 1 to 30 drug molecules per antibody or antigen-binding fragment thereof).

### Therapeutic Formulation and Administration

The present invention provides pharmaceutical compositions comprising the antigen-binding molecules of the present invention. The pharmaceutical compositions of the invention are formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of antigen-binding molecule administered to a patient may vary depending upon the age and the size of the patient, target disease, conditions, route of administration, and the like. The preferred dose is typically calculated according to body weight or body surface area. When a bispecific antigen-binding molecule of the present invention is used for therapeutic purposes in an adult patient, it may be advantageous to intravenously administer the bispecific antigen-binding molecule of the present invention normally at a single dose of about 0.01 to about 20 mg/kg body weight, more preferably about 0.02 to about 7, about 0.03 to about 5, or about 0.05 to about 3 mg/kg body weight. Depending on the severity of the condition, the frequency and the duration of the treatment can be adjusted. Effective dosages and schedules for administering a bispecific antigen-binding molecule may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present invention include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.), and the HUMIRA™ Pen (Abbott Labs, Abbott Park IL), to name only a few.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, it is preferred that the aforesaid antibody is contained in about 5 to about 100 mg and in about 10 to about 250 mg for the other dosage forms.

Therapeutic and Diagnostic Uses thereof

Disclosed herein are also methods comprising administering to a subject in need thereof a therapeutic composition comprising an anti-CD63 antibody or antigen-binding fragment thereof (including a multispecific binding molecule or multidomain therapeutic protein comprising same) or an antibody-drug conjugate comprising an anti-CD63 antibody (e.g., an anti-CD63 antibody or ADC comprising any of the HCVR/LCVR or CDR sequences as set forth in Table 1 herein). The therapeutic composition can comprise any of the anti-CD63 antibodies, antigen-binding fragments thereof, or ADCs disclosed herein, and a pharmaceutically acceptable carrier or diluent.

The antibodies, antigen-binding fragment thereof (including a multispecific binding molecule or multidomain therapeutic protein comprising same) or an antibody-drug conjugate comprising an anti-CD63 antibody of the invention may be useful, inter alia, for the treatment, prevention and/or amelioration of any disease or disorder associated with or mediated by CD63. For example, the antibodies and ADCs of the present invention are useful for the treatment of tumors that express CD63, and in some embodiments a TAA which may be targeted by a bispecific antibody of the invention. The antibodies, antigen-binding fragment thereof (including a multispecific binding molecule or multidomain therapeutic protein comprising same) or an antibody-drug conjugate comprising an anti-CD63 antibody of the invention may also be useful in treating mast cell (MC-) dependent diseases, rheumatoid arthritis, IgE-dependent allergic reactions and Fc-ER1-mediated allergic reaction, asthma, cancer and/or metastases, etc. See, e.g., Kraft et al. (2005) JEM 201:385; Valadi (2007) Nat. Cell Biol. 9:654

The anti-CD63 antibodies of the invention have various utilities. In one embodiment, the anti-CD63 antibodies are useful for the affinity purification of exosomes, e.g., in which the anti-CD63 antibodies disclosed herein can be immobilized on a suitable support, such as a Sephadex resin or filter paper, using methods well known in the art, and then contacted with a sample containing the desired exosomes containing CD63 to be purified, and thereafter the support is washed with a suitable solvent that will remove substantially all the material in the sample except the exosomes which are bound to the immobilized antibody. Finally, the support can be washed with another suitable solvent that will release the exosomes from the antibody. In another embodiment, anti-CD63 antibodies may be used in diagnostic assays for CD63, e.g., detecting its expression in specific cells, tissues, or serum, e.g., as a reagent to identify/label exosomes. See, e.g., Valadi et al. (2007) Nature Cell. Biol: 9(6):654-9. Various diagnostic and prognostic assay techniques known in the art may be used, such as competitive binding assays, direct or indirect sandwich assays and immunoprecipitation assays conducted in either heterogeneous or homogeneous phases (Zola (1987) Monoclonal Antibodies: A Manual of Techniques, CRC Press, Inc. pp. 147-1581). The antibodies used in the assays can be labeled with a detectable moiety. The detectable moiety should be capable of producing, either directly or indirectly, a detectable signal. Any method known in the art for conjugating the antibody to the detectable moiety may be employed.

In another embodiment, provided is a method of treatment of a disease, such as cancer. The method of the invention preferably includes the step of providing an antibody or CD63 antigen-binding fragment thereof, as described above, to a subject requiring said treatment.

Methods of immunotargeting cancer cells using antibodies or antibody fragments are well known in the art. U.S. Pat. No. 6,306,393, for instance, describes the use of anti-CD22 antibodies in the immunotherapy of B-cell malignancies, and U.S. Pat. No. 6,329,503 describes immunotargeting of cells that express serpentine transmembrane antigens. Antibodies described herein (including humanized or human monoclonal antibodies or fragments or other modifications thereof, optionally conjugated to cytotoxic or other agents) can be introduced into a patient such that the antibody binds to cancer cells and mediates the destruction of the cells and the tumor and/or inhibits the growth of the cells or the tumor.

Without intending to limit the disclosure, mechanisms by which such antibodies can exert a therapeutic effect may include, for example, complement-mediated cytolysis, antibody-dependent cellular cytotoxicity (ADCC)1 modulating the physiologic function of the tumor antigen, inhibiting binding or signal transduction pathways, modulating tumor cell differentiation, altering tumor angiogenesis factor profiles, modulating the secretion of immune stimulating or tumor suppressing cytokines and growth factors, modulating cellular adhesion, and/or by inducing apoptosis.

### Example 1: Exemplary CD63 Antibodies

### Generation of Anti-Human CD63 Antibodies

Anti-human CD63 antibodies were obtained by immunizing a mouse (e.g., an engineered mouse comprising DNA encoding human immunoglobulin heavy and human kappa light chain variable regions), with human CD63.

Following immunization, splenocytes were harvested from each mouse and either (1) fused with mouse myeloma cells to preserve their viability and form hybridoma cells and screened for human CD63 specificity, or (2) B-cell sorted (as described in US 2007/0280945A1) using a either a human CD63 fragment as the sorting reagent that binds and identifies reactive antibodies (antigen-positive B cells).

Chimeric antibodies to human CD63 were initially isolated having a human variable region and a mouse constant region using, e.g., VELOCIMMUNE technology as described in U.S. Pat. Nos. 7,105,348; 8,642,835; and 9,622,459, each of which is incorporated herein by reference.

In some antibodies, for testing purposes, mouse constant regions were replaced with a desired human constant region, for example wild-type human C_{H} or modified human C_{H} (e.g. IgG1, IgG2 or IgG4 isotypes), and light chain constant region (C_{L}), to generate a fully human anti-hCD63, including a fully human bispecific antibody comprising an anti-hCD63antibody or antigen binding portion thereof. While the constant region selected may vary according to specific use, high affinity antigen-binding and target specificity characteristics reside in the variable region.

Certain biological properties of the exemplary bispecific antibodies comprising an anti-human CD63 binding arm generated in accordance with the methods of this Example are described in detail in the Examples set forth below.

### Heavy and Light Chain Variable Region Amino Acid and Nucleic Acid Sequences of Anti-CD63 Antibodies

Table 1 sets forth sequence identifiers of a nucleic acid (NA) sequence encoding, and in parentheses an amino acid (AA) sequence of, a heavy or light chain variable region (HCVR or LCVR, respectively), or a heavy or light chain CDR (HCDR and LCDR, respectively) of selected anti-CD63 antibodies used to generate the multidomain therapeutic anti-CD63 proteins disclosed herein.

TABLE 1
anti-CD63 Sequence Identifiers

| | SEQ ID NOs: | | | | | | | |
|---|---|---|---|---|---|---|---|---|
| | HCVR | HCDR1 | HCDR2 | HCDR3 | LCVR | LCDR1 | LCDR2 | LCDR3 |
| Antibody | NA | NA | NA | NA | NA | NA | NA | NA |
| Designation | (AA) | (AA) | (AA) | (AA) | (AA) | (AA) | (AA) | (AA) |
| H1M12451N | 1 | 3 | 5 | 7 | 9 | 11 | 13 | 15 |
| | (2) | (4) | (6) | (8) | (10) | (12) | (14) | (16) |
| H2M12395N | 17 | 19 | 21 | 23 | 25 | 27 | 29 | 31 |
| | (18) | (20) | (22) | (24) | (26) | (28) | (30) | (32) |
| H4H12450N | 33 | 35 | 37 | 39 | 41 | 43 | 45 | 47 |
| | (34) | (36) | (38) | (40) | (42) | (44) | (46) | (48) |
| H2M12450N | 49 | 51 | 53 | 55 | 57 | 59 | 61 | 63 |
| | (50) | (52) | (54) | (56) | (58) | (60) | (62) | (64) |
| H1M12362N | 65 | 67 | 69 | 71 | 73 | 75 | 77 | 79 |
| | (66) | (68) | (70) | (72) | (74) | (76) | (78) | (80) |
| H1M12366N | 81 | 83 | 85 | 87 | 89 | 91 | 93 | 95 |
| | (82) | (84) | (86) | (88) | (90) | (92) | (94) | (96) |
| H1M12386N | 97 | 99 | 101 | 103 | 105 | 107 | 109 | 111 |
| | (98) | (100) | (102) | (104) | (106) | (108) | (110) | (112) |
| H1M12388N | 113 | 115 | 117 | 119 | 121 | 123 | 125 | 127 |
| | (114) | (116) | (118) | (120) | (122) | (124) | (126) | (128) |
| H1M12390N | 129 | 131 | 133 | 135 | 137 | 139 | 141 | 143 |
| | (130) | (132) | (134) | (136) | (138) | (140) | (142) | (144) |
| H2M12385N | 145 | 147 | 149 | 151 | 153 | 155 | 157 | 159 |
| | (146) | (148) | (150) | (152) | (154) | (156) | (158) | (160) |
| H2M12387N | 161 | 163 | 165 | 167 | 169 | 171 | 173 | 175 |
| | (162) | (164) | (166) | (168) | (170) | (172) | (174) | (176) |
| H2M12392N | 177 | 179 | 181 | 183 | 185 | 187 | 189 | 191 |
| | (178) | (180) | (182) | (184) | (186) | (188) | (190) | (192) |
| H2M12394N | 193 | 195 | 197 | 199 | 201 | 203 | 205 | 207 |
| | (194) | (196) | (198) | (200) | (202) | (204) | (206) | (208) |
| H2M12452N | 209 | 211 | 213 | 215 | 217 | 219 | 221 | 223 |
| | (210) | (212) | (214) | (216) | (218) | (220) | (222) | (224) |
| H2M12454N | 225 | 227 | 229 | 231 | 233 | 235 | 237 | 239 |
| | (226) | (228) | (230) | (232) | (234) | (236) | (238) | (240) |
| H2M13022N | 241 | 243 | 245 | 247 | 249 | 251 | 253 | 255 |
| | (242) | (244) | (246) | (248) | (250) | (252) | (254) | (256) |
| H3M12361N | 257 | 259 | 261 | 263 | 265 | 267 | 269 | 271 |
| | (258) | (260) | (262) | (264) | (266) | (268) | (270) | (272) |
| H4H11990P2 | 273 | 275 | 277 | 279 | 281 | 283 | 285 | 287 |
| | (274) | (276) | (278) | (280) | (282) | (284) | (286) | (288) |
| H4H11992P2 | 289 | 291 | 293 | 295 | 281 | 283 | 285 | 287 |
| | (290) | (292) | (294) | (296) | (282) | (284) | (286) | (288) |
| H4H11993P2 | 297 | 299 | 301 | 303 | 281 | 283 | 285 | 287 |
| | (298) | (300) | (302) | (304) | (282) | (284) | (286) | (288) |
| H4H11995P2 | 305 | 307 | 309 | 311 | 281 | 283 | 285 | 287 |
| | (306) | (308) | (310) | (312) | (282) | (284) | (286) | (288) |
| H4H11996P2 | 313 | 315 | 317 | 319 | 281 | 283 | 285 | 287 |
| | (314) | (316) | (318) | (320) | (282) | (284) | (286) | (288) |
| H4H11997P2 | 321 | 323 | 325 | 327 | 281 | 283 | 285 | 287 |
| | (322) | (324) | (326) | (328) | (282) | (284) | (286) | (288) |
| H4H11998P2 | 329 | 331 | 333 | 335 | 281 | 283 | 285 | 287 |
| | (330) | (332) | (334) | (336) | (282) | (284) | (286) | (288) |

SEQ ID NO: 13:
GGTGTATTC
SEQ ID NO: 14:
GVF
SEQ ID NO: 29:
GGTGGATCC
SEQ ID NO: 30:
GGS
SEQ ID NO: 45:
GGTGCATCC
SEQ ID NO: 46:
GAS
SEQ ID NO: 61:
GGTGCATCC
SEQ ID NO: 62:
GAS
SEQ ID NO: 77:
GCTGCATCC
SEQ ID NO: 78:
AAS
SEQ ID NO: 93:
GGTGCATCC
SEQ ID NO: 94:
GAS
SEQ ID NO: 109:
GTAGCATCC
SEQ ID NO: 110:
VAS
SEQ ID NO: 125:
GGTGCATCC
SEQ ID NO: 126:
GAS
SEQ ID NO: 141:
GCTGCATCC
SEQ ID NO: 142:
AAS
SEQ ID NO: 157:
GCTGCATCC
SEQ ID NO: 158:
AAS
SEQ ID NO: 173:
GGTGCATCC
SEQ ID NO: 174:
GAS
SEQ ID NO: 189:
GGTGCATCC
SEQ ID NO: 190:
GAS
SEQ ID NO: 205:
GGTGCATCC
SEQ ID NO: 206:
GAS
SEQ ID NO: 221:
GGTCCATCC
SEQ ID NO: 222:
GPS
SEQ ID NO: 237:
GGTGCATCC
SEQ ID NO: 238:
GAS
SEQ ID NO: 253:
GCTGCATCC
SEQ ID NO: 254:
AAS
SEQ ID NO: 269:
GCTGCATCC
SEQ ID NO: 270:
AAS
SEQ ID NO: 285:
GCTGCATCC
SEQ ID NO: 286:
AAS

### Binding by Parental Anti-Human CD63 Antibodies

Relative cell surface binding of the anti-CD63 antibodies to human CD63 expressing cells was accessed via flow cytometry using CD63 positive HEK293 cells (ATCC, Cat #CRL-1573), which endogenously express human CD63, and CD63 negative HEK293/CD63 knock out cells. For the assay, cells were plated in PBS without calcium and magnesium (VWR, Cat #45000-446), containing 2% FBS (Saradigm Cat #1500-500) (Staining Buffer) in 96 well V-bottom plates (Axygen Scientific, Cat #P-96-450-V-C-S). Cells were then incubated with anti-CD63 antibodies or isotype control antibodies at concentrations ranging from 100 nM to 1.7 μM for 30 minutes on ice. Wells containing no antibody were used as controls. HEK293/CD63KO cells were stained with only the highest concentration (100 nM) of the antibodies. The cells were then washed once with staining buffer and were incubated with a PE conjugated anti-mouse Fc secondary antibody (Jackson ImmunoResearch, Cat #115-115-164) at 100 nM for 30 minutes at 4° C. Cells were then washed and fixed using a 50% solution of Cytofix (BD Biosciences, Cat #554655) diluted in PBS. Samples were run on the Intellicyte Hypercyt flow cytometer and results were analyzed in ForeCyt software (Intellicyte) to calculate the mean fluorescent intensity (MFI). Measured values were analyzed using a four parameter logistic equation over a 12-point response curve using GraphPad Prism and the resulting EC₅₀ values are reported (Table 12). The signal to noise ratio (S/N) was determined by calculating the ratio of the anti-CD63 antibodies or the control antibodies MFI to the wells containing no antibodies (Table 12).

As shown in Table 2, three of the anti-CD63 antibodies of the invention demonstrated binding to HEK293 cells with S/N values ranging from 21.0 to 31.6 and EC₅₀ values ranging from 0.5 nM to 1.9 nM. The non-binding controls did not demonstrate binding to HEK293 cells (S/N<1.5). Both the anti-CD63 antibodies and the isotype control antibodies demonstrated weak to little binding to the HEK293/CD63KO cells (S/N<4.4).

TABLE 2
Binding of anti-CD63 antibodies to HEK293 and HEK293/CD63
KO cells as measured by flow cytometry

| | HEK293 EC₅₀ | HEK293 | HEK293/CD63 KO |
|---|---|---|---|
| Antibody | (nM) | (S/N) | (S/N) |
| H2M12450N | 0.5 | 26.7 | 2.9 |
| H1M12451N | 1.8 | 31.6 | 4.1 |
| H2M12395N | 1.9 | 21.0 | 2.9 |
| Isotype Control 1 | ND | 1.4 | 4.4 |
| Isotype Control 2 | ND | 1.5 | 1.6 |

ND = Not determined

The ability of the anti-CD63 monoclonal antibodies of the invention to bind to human CD63 expressing cells was also determined using an electrochemiluminescence (ECL) based detection assay.

To generate overexpressing cells, mouse embryonic fibroblast NIH3T3 cells (ATCC, Cat #CRL-1658) were transfected to form a cell line “NIH3T3/hCD63” that stably expresses human CD63 (hCD63; amino acids M1-M238 of accession number NP_001771; SEQ ID NO: 337). Expression levels of human CD63 in endogenously expressing cells, a human androgen-sensitive prostate adenocarcinoma cell line, LNCAP (ATCC, Cat #CRL-1740), and human primary glioblastoma cell line, U87MG (ATCC, Cat #HTB-14) were analyzed with a Quantum™ Alexa Fluor© 647 MESF (Bangs Laboratories, Cat #647B) and a Simply Cellular® anti-Mouse IgG (Bangs Laboratories Inc, Cat #815) following the manufacturer's instructions. LNCAP cells were determined to have a lower human CD63 copy number than U87MG cells. Non-transfected NIH3T3 cells, which have no detectable expression of human CD63 by fluorescence activated cell sorting (FACS), were included as a negative control.

Briefly, cell lines were rinsed once in PBS buffer without Ca²⁺/Mg²⁺ and incubated for 10 minutes at 37° C. with Enzyme Free Cell Dissociation Solution (Millipore, Cat #S-004-C) to detach the cells. Cells were then washed once with PBS with Ca²⁺/Mg²⁺ and counted with a Cellometer™ Auto T4 cell counter (Nexcelom Bioscience, LLC). Approximately 2.0×10⁴ NIH3T3/hCD63, LNCAP, U87MG or NIH3T3 cells were seeded separately onto 96-well carbon electrode plates (Meso Scale Discovery, Cat #L15XB-6) and were then incubated for one hour at 37° C. Nonspecific binding sites were blocked with 2% BSA (w/v) in PBS with Ca²⁺/Mg²⁺ for one hour at room temperature (RT). Solutions containing anti-CD63 antibodies or isotype control antibodies at a range of concentrations (1.7 μM to 100 nM) in 0.5% BSA (w/v) in PBS with Ca²⁺/Mg²⁺, as well as control buffer alone, were then added in duplicate to the plate-bound NIH3T3/hCD63, LNCAP, U87MG or NIH3T3 cells and incubated for one hour at RT. Plates were subsequently washed to remove unbound antibodies using an AquaMax2000 plate washer with a cell washing head (MDS Analytical Technologies). The plate-bound antibodies were detected with 1p g/mL of either a SULFO-TAG™-conjugated goat polyclonal anti-human IgG antibody specific for Fcγ fragment (Jackson Immunoresearch, Cat #109-005-098) or a SULFO-TAG™-conjugated goat polyclonal anti-mouse IgG antibody specific for Fcγ fragment (Jackson Immunoresearch, Cat #115-005-164) for one hour at RT. Plates were washed and then incubated with Read Buffer (MSD, Cat #R92TD-2) according to the manufacturer's instructions. Luminescent signals were measured using a SECTOR Imager (MSD). Luminescence intensity, measured in relative light units (RLU), was recorded to indicate the binding intensity of each antibody at the range of concentrations tested. The ratio of signal detected with 3.7 nM antibody binding to human CD63 expressing cells compared to the same concentration of antibody binding to negative cells was reported as an indication of specificity of CD63 binding. Antibodies with the binding ratio of greater than 3 were classified as specific binders and antibodies with the binding ratio less than or equal to 3 were classified as non-binders and marked as NB in Table 13. In addition, the direct binding signals (in RLU) were analyzed as a function of the antibody concentration and the data were fitted with a sigmoidal (four-parameter logistic) dose-response model using GraphPad Prism™ software. The EC₅₀ value for binding to human CD63 expressing cells, defined as the concentration of antibody at which 50% of the maximal binding signal is detected, was determined to indicate potency of each antibody and reported in Table 3 only for specific binders. The data for two separate experiments testing different antibodies are reported in Tables 3A and 3B.

As shown in Table 3A, the four anti-CD63 antibodies generated as described in Exathis Example and a Comparator Ab (Comparator 1) bound specifically to human CD63 expressed on engineered NIH3T3/hCD63 cells as well as that endogenously expressed on LNCAP and U87MG cell lines. The four anti-CD63 antibodies described in this Example bound to NIH3T3/hCD63 cells with EC₅₀ values ranging from 280 μM to 970 μM and binding ratios over the negative cell line ranging from 91 to 281-fold. The four anti-CD63 antibodies of the invention bound to U87MG cells with EC₅₀ values ranging from 500 μM to 1.4 nM and binding ratios over the negative cell line ranging from 52 to 272-fold. The four anti-CD63 antibodies generated as described in this Example bound to LNCAP cells with EC₅₀ values ranging from 210 μM to 1.7 nM and binding ratios over the negative cell line ranging from 7 to 20-fold. The lower binding ratios on LNCAP cells are in agreement with lower CD63 copy number on these cells compared to U87MG cells. The isotype control antibodies were non-binders, as expected, with cell binding ratios less than or equal to 3.

TABLE 3A
Anti-CD63 Antibodies Binding to Human CD63 expressing cells
as measured by Electrochemiluminescence based detection

| | | | | Ratio at 3.7 nM Ab concentration of | | |
|---|---|---|---|---|---|---|
| | | | | Cell Binding Signal (RLU) to | | |
| | | | | human CD63 cells relative to | | |
| | Cell Binding Potency, EC₅₀ (M) | | | negative NIH3T3 | | |
| Antibody | NIH3T3/hCD63 | U87MG | LNCAP | NIH3T3/hCD63 | U87MG | LNCAP |
| H1M12451N | 2.9E−10 | 7.9E−10 | 3.5E−10 | 256 | 171 | 18 |
| H2M12395N | 5.7E−10 | 9.5E−10 | 1.7E−09 | 91 | 52 | 7 |
| H4H12450N | 2.8E−10 | 5.0E−10 | 2.1E−10 | 230 | 175 | 20 |
| H2M12450N | 9.7E−10 | 1.4E−09 | 1.4E−09 | 281 | 272 | 20 |

CONTROLS

| | | | | | | |
|---|---|---|---|---|---|---|
| Comparator | 5.7E−10 | 6.7E−10 | 2.0E−09 | 253 | 271 | 16 |
| Ab 1 | | | | | | |
| Human IgG4 | NB | NB | NB | 1 | 1 | 1 |
| isotype control | | | | | | |
| Mouse IgG2 | NB | NB | NB | 3 | 2 | 2 |
| isotype control | | | | | | |

NB-non-binder;
antibodies with a binding ratio of less than or equal to 3 were classified as non-binders.

As shown in Table 3B, nineteen of the twenty anti-CD63 antibodies tested, as well as a Comparator Ab (Comparator 1), bound specifically to human CD63 expressed on engineered NIH3T3/hCD63 cells and on endogenously expressing U87MG and LNCAP cell lines. One anti-CD63 antibody, H4H11998P2, specifically bound to NIH3T3/hCD63 and U87MG, but did not bind to LNCAP. The twenty anti-CD63 antibodies tested bound to NIH3T3/hCD63 cells with EC50 values ranging from 0.320 nM to 7.4 nM with binding ratios over the negative cell line ranging from 14 to 608-fold and bound to U87MG cells with EC50 values ranging from 0.29 nM to 17 nM with binding ratios ranging from 5 to 405-fold. Nineteen anti-CD63 antibodies bound to LNCAP cells with EC50 values ranging from 0.15 μM to 55 nM with binding ratios over the negative cell line ranging from 4 to 51-fold. The one anti-CD63 antibody, H4H11998P2, was non-binder to LNCAP cells with a cell binding ratio of 1. The lower binding ratios on LNCAP cells are in agreement with lower CD63 copy number on these cells compared to U87MG cells. The isotype control antibodies were non-binders, as expected, with cell binding ratios less than or equal to 3 or binding signal less than 100 RLU on the human CD63 cell lines tested.

TABLE 3B
Anti-CD63 Antibodies Binding to Human CD63 expressing cells
as measured by Electrochemiluminescence based detection

| | | | | Ratio at 3.7 nM Ab concentration of | | |
|---|---|---|---|---|---|---|
| | | | | Cell Binding Signal (RLU) to | | |
| | | | | human CD63 cells relative to | | |
| | Cell Binding Potency, EC₅₀ (M) | | | negative NIH3T3 | | |
| Antibody | NIH3T3/hCD63 | U87MG | LNCAP | NIH3T3/hCD63 | U87MG | LNCAP |
| H2bM12454N | 3.2E−10 | 4.7E−10 | 1.8E−10 | 175 | 132 | 14 |
| H2aM12394N | 3.8E−10 | 1.4E−09 | 7.5E−10 | 61 | 50 | 4 |
| H2aM12387N | 3.9E−10 | 2.9E−10 | 1.5E−10 | 228 | 172 | 18 |
| H2aM12392N | 4.0E−10 | 5.3E−10 | 2.3E−10 | 608 | 405 | 51 |
| H2bM12385N | 4.5E−10 | 6.4E−10 | 2.9E−10 | 71 | 47 | 5 |
| H2bM13022N | 4.5E−10 | 8.5E−10 | 3.2E−10 | 95 | 71 | 8 |
| H1M12390N | 4.8E−10 | 3.6E−10 | 3.0E−10 | 559 | 339 | 31 |
| H1M12388N | 5.2E−10 | 3.0E−10 | 2.5E−10 | 434 | 284 | 31 |
| H1M12386N | 5.2E−10 | 3.1E−10 | 2.9E−10 | 220 | 129 | 16 |
| H2aM12452N | 6.1E−10 | 2.0E−09 | 7.4E−10 | 593 | 260 | 37 |
| H1M12362N | 6.3E−10 | 5.6E−10 | 5.7E−10 | 283 | 174 | 16 |
| H3M12361N | 8.1E−10 | 1.3E−09 | 1.1E−09 | 243 | 111 | 19 |
| H3M12366N | 8.3E−10 | 2.0E−09 | 1.8E−09 | 310 | 107 | 7 |
| H4H11990P2 | 9.3E−10 | 4.8E−09 | 5.1E−09 | 168 | 66 | 6 |
| H4H11997P2 | 9.4E−10 | 1.7E−09 | 1.9E−09 | 266 | 99 | 11 |
| H4H11996P2 | 9.8E−10 | 8.1E−09 | 4.4E−09 | 188 | 57 | 7 |
| H4H11992P2 | 1.5E−09 | 5.5E−09 | 3.6E−09 | 87 | 33 | 4 |
| H4H11993P2 | 2.5E−09 | 1.7E−08 | 6.7E−09 | 227 | 62 | 8 |
| H4H11995P2 | 5.0E−09 | 1.3E−08 | 5.5E−08 | 212 | 61 | 5 |
| H4H11998P2 | 7.4E−09 | IC | NB | 14 | 5 | 1 |

CONTROLS

| | | | | | | |
|---|---|---|---|---|---|---|
| Comparator | 9.6E−10 | 1.1E−09 | 5.3E−10 | 589 | 243 | 25 |
| Ab 1 | | | | | | |
| Human IgG4 | NB | NB | NB | 1 | 4(*) | 1 |
| isotype control | | | | | | |
| Mouse IgG2 | NB | NB | NB | 3 | 2 | 2 |
| isotype control | | | | | | |

NB-non-binder;
antibodies with a binding ratio of less than or equal to 3 or binding signal of less than 100 RLU.
(*)-binding signal on U87MG was less than 100 RLU and isotype control was classified as non-binder.
IC-inconclusive; no sigmoidal fit observed to calculate EC₅₀ value.

### Internalization/Cytotoxicity Mediated by Parental Anti-Human CD63 Antibodies

The ability of the anti-CD63 antibodies disclosed herein to bind and internalize on CD63 expressing cells was assessed. For the assay, T47D cells (ATCC, Cat #HTB-133) were seeded into 96 well Collagen coated plates (Greiner, Cat #655956) in RPMI (Irvine Scientific, Cat #9160) containing 10% FBS (ATCC, Cat #30-2020), penicillin/streptomycin/L-glutamine (Gibco, Cat #10378-016), 100 μM sodium pyruvate (Millipore, Cat #TM5-005-C), 1 mM HEPES (ThermoFisher, Cat #15630080), 10 μg/mL Insulin bovine (Gemini BioProducts, Cat #700-912P) (growth media) and allowed to incubate overnight at 37° C. in 5% CO₂. To stain, quadruplicate plates of cells were incubated with 10 μg/mL of anti-CD63 antibodies that was diluted in 2% FBS in PBS, without Calcium and Magnesium (Irving, Cat #9240) (staining buffer) for 30 minutes at 4° C. Cells were washed twice with staining buffer, then incubated with an Alexa-Flour 488 conjugated secondary Ab (Jackson Immunoresearch, Cat #115-547-003 or Jackson Immunoresearch, Cat #109-547-003) at 10 μg/mL for 30 minutes at 4° C., and subsequently washed twice more with staining buffer. Two plates were immediately fixed and stained with 4% paraformaldehyde (PFA; ThermoFisher, Cat #28908)+5 μM DRAQ5 (ThermoFisher, Cat #62251) in PBS for 20 minutes (non-internalization plates). The remaining two plates were incubated with growth media at 37° C. for 2 hours followed by fixation and staining for 20 minutes using a solution of 4% PFA+5 μM DRAQ5 diluted in PBS (internalization plates). After fixation, all plates were washed once with PBS. One non-internalization plate and one internalization plate were incubated with an anti-Alexa Fluor 488 antibody (Regeneron) at 50 μg/mL in PBS overnight at 4° C. to quench surface Alexa Fluor 488 fluorescence. The remaining plates were incubated with PBS only. Confocal images were acquired on the Opera Phenix (Perkin Elmer) at 40× magnification. Harmony analysis software (Perkin Elmer) was utilized to identify DRAQ5-labeled cells and the total Alexa-Fluor 488 relative fluorescent units (RFU) per cell was determined. The total binding at 4° C. (RFU values of 4° C. unquenched wells), total binding at 37° C. (RFU values of 37° C. unquenched wells), the total internalized RFU, and the % Internalization were determined for each antibody as shown in Table 1. For all calculations, background fluorescence from 2^{nd} Ab only control wells were subtracted from every well. Total internalized RFU was calculated as follows: Total RFU of 37° C. unquenched samples—Surface RFU at 37° C. Surface RFU is defined as unquenched RFU at 37° C.-quenched RFU at 37° C.)/QE. QE (quenching efficiency) is defined as: 1-(Total RFU of 4° C. quenched sample/Total RFU of 4° C. unquenched sample). The % Internalization was determined from the following formula: (Total internalized RFU at 37° C./Total RFU at 37° C.)*100.

As shown in Table 4, all 4 anti-CD63 antibodies of the invention demonstrated internalization into T47D cells ranging from 60.9% to 73.7% internalization. The isotype controls did not demonstrate any measurable internalization.

TABLE 4
Internalization and Surface Binding
of anti-CD63 Antibodies in T47D Cells

| | | | Total | % |
|---|---|---|---|---|
| | Total RFU | Total RFU at | Internalized | Internal- |
| Antibody | at 4° C. | 37° C. (2 h) | RFU | ization |
| H4H12450N | 2098644 | 2083920 | 1419840 | 68.1 |
| hIgG4 Isotype | 137648 | −80525 | −75133 | ND* |
| control | | | | |
| H2aM12450N | 2359553 | 2938050 | 1790248 | 60.9 |
| H1M12451N | 1902759 | 2321763 | 1710839 | 73.7 |
| H2M12395N | 1499634 | 1693000 | 1238752 | 73.2 |
| mIgG1 Isotype | −93885 | 57417 | 66563 | ND* |
| control | | | | |
| mIgG2a Isotype | −73427 | 65365 | 73850 | ND* |
| control | | | | |

ND*: % internalization could not be determined due to weak binding and/or inability to determine quenching efficiency

In order to assess the ability of an anti-CD63 antibody described herein to internalize in CD63 expressing cells, an in vitro indirect cytotoxicity assay was performed. Human CD63 positive T47D cells (ATCC, Cat #HTB-133) and human CD63 negative NIH3T3 cells (ATCC, Cat #CRL-1658) were respectively seeded in PDL-coated 96-well plates (BD Biocoat, Cat #356461) at either 6,000 cells per well in RPMI (Irvine Scientific, Cat #9160) containing 10% FBS (ATCC, Cat #30-2020), penicillin/streptomycin/L-glutamine (Gibco, Cat #10378-016), 50 uM Beta-Mercaptoethanol (Sigma, Cat #M7522) (growth media), Sodium Pyruvate 100 mM (Millipore, Cat #TMS-005-C), HEPES 1M (Irvine Scientific, Cat #9319), and Insulin bovine 10 μg/mL (Gemini BioProducts, Cat #700-912P) or 2,000 cells per well in DME high glucose (Irvine Scientific, Cat #9033), 10% Bovine calf serum (Hyclone, Cat #SH30072.03), plus penicillin/streptomycin/L-glutamine (Gibco, Cat #10378-016) and grown overnight at 37° C. in 5% CO₂. For cell viability curves, cells were incubated for 5 minutes at 37° C. with a serially diluted anti-CD63 antibody (H2M12450N) or a non-binding isotype control antibody at concentrations ranging from 3.0 μM to 2.2 nM. A Fab anti-mFc secondary antibody conjugated to the cytotoxic payload MMAF (Moradec, Cat #AM-201AF-50) was then added at 20 nM to each well. Media alone served as a negative control, and 33 μM of digitonin (Promega, Cat #G9441) was used to determine the maximum cytotoxicity. Following a 72 hour incubation, cell viability was measured using Cell Counting Kit-8 (Dojindo, Cat #CK04) as per manufacturer's protocols with an incubation time range of 1-3 hours. The absorbance at 450 nm (OD₄₅₀) was measured on an Envision plate reader (PerkinElmer). Background OD₄₅₀ levels from digitonin treated cells were subtracted from all wells and viability was expressed as a percentage of the untreated controls (% viability). IC₅₀ values were determined from a four-parameter logistic equation over an 8-point response curve (GraphPad Prism). All IC₅₀ values are expressed in nM concentration and the minimum % viable cells remaining after treatment is reported.

As summarized in Table 5, the anti-CD63 antibody, H2M12450N, reduced T47D viability to 21% with an IC₅₀ value of 0.24 nM, whereas the isotype control reduced viability to only 64%. The antibodies had little to no impact on the viability of the NIH3T3 cell line.

TABLE 5
Anti-CD63 antibody internalization measured by an indirect
cytotoxicity assay in T47D and NIH3T3 cells

| | T47D | | NIH3T3 | |
|---|---|---|---|---|
| | (nM) | T47D % | (nM) | NIH3T3% |
| Antibody | IC₅₀ | Viability | IC₅₀ | Viability |
| H2M12450N | 0.24 | 21 | ND | 83 |
| Isotype Control | ND | 64 | ND | 91 |

ND = Not determined

### Biacore Binding Kinetics of Anti-CD63 Monoclonal Antibodies Binding to CD63 (EC2) Loop Reagents Measured at 25° C. And 37° C.

Equilibrium dissociation constants (K_{D}) for different CD63 (EC2) loop reagents binding to purified anti-CD63 monoclonal antibodies were determined using a real-time surface plasmon resonance based Biacore T200 biosensor or Biacore 2000 biosensor. All binding studies were performed in 10 mM HEPES, 150 mM NaCl, 3 mM EDTA, and 0.05% v/v Surfactant Tween-20, pH 7.4 (HBS-ET) running buffer at 25° C. and 37° C. The Biacore CM5 sensor chip surface was first derivatized by amine coupling with either a rabbit anti-mouse Fc specific polyclonal antibody (GE Healthcare, Cat #BR100838) or an anti-human Fab kit (GE Healthcare, Cat #28958325) to capture anti-CD63 monoclonal antibodies. Binding studies were performed on either recombinant human CD63 extracellular loop 2 expressed with a C-terminal Myc-Myc-hexahistidine (hCD63 EC loop 2-MMH; SEQ ID NO: 338) or recombinant human CD63 extracellular loop 2 expressed with a C-terminal human Fe tag (hCD63 EC loop 2-hFc; SEQ ID NO: 339). Different concentrations of hCD63 EC loop 2-MMH or hCD63 EC loop 2-hFc (either tested at 50 nM 12.5 nM in a 4-fold dilution or at 90 nM-0.37 nM in 3-fold serial dilutions) were first prepared in HBS-ET running buffer and were injected over the captured anti-CD63 monoclonal antibody surface for 4 minutes at a flow rate of 35 μL/minute or 50 μL/minute, while the dissociation of monoclonal antibody bound CD63 reagent was monitored for 8 or 10 minutes in HBS-ET running buffer. The association rate (kₐ) and dissociation rate (k_{d}) were determined by fitting the real-time binding sensorgrams to a 1:1 binding model with mass transport limitation using Scrubber 2.0c curve-fitting software. Binding dissociation equilibrium constant (K_{D}) and dissociative half-life (t½) were calculated from the kinetic rates as:

$${K}_{D} ( M ) = \frac{kd}{ka} , and t \frac{1}{2} ( min ) = \frac{ln ( 2 )}{60 * kd}$$

Binding kinetics parameters for human CD63 EC loop 2 protein binding to different anti-CD63 monoclonal antibodies of the invention at 25° C. and 37° C. are shown in Tables 6 through 9.

At 25° C., 20 of 23 of the anti-CD63 monoclonal antibodies of the invention bound to human CD63 EC loop 2-MMH with K_{D} values ranging from 676 μM to 11.7 uM, as shown in Table 6. At 37° C., 21 of 23 of the anti-CD63 monoclonal antibodies of the invention bound to human CD63 EC loop 2-MMH with K_{D} values ranging from 1.15 nM to 12.3 μM, as shown in Table 7. At 25° C., 22 of 23 of the anti-CD63 monoclonal antibodies of the invention bound to human CD63 EC loop 2-Fc with K_{D} values ranging from 129 μM to 10.1 nM, as shown in Table 8. At 37° C., 22 of 23 of the anti-CD63 monoclonal antibodies of the invention bound to human CD63 EC loop 2-Fc with K_{D} values ranging from 45.0 pM to 14.5 nM, as shown in Table 9.

TABLE 6
Binding kinetics parameters of human CD63 EC loop 2-MMH
binding to anti-CD63 monoclonal antibodies at 25° C.

| | mAb | | | | | |
|---|---|---|---|---|---|---|
| | Capture | 100 nM Ag | | | | |
| | Level | Bound | kₐ | k_{d} | K_{D} | t½ |
| Antibody | (RU) | (RU) | (1/Ms) | (1/s) | (M) | (min) |
| H2aM12450N | 592 | 65 | 2.27E+05 | 2.79E−04 | 1.23E−09 | 41 |
| H2aM12395N | 472 | 42 | 1.66E+05 | 1.82E−03 | 1.10E−08 | 6 |
| H1M12451N | 525 | 52 | 8.59E+04 | 4.30E−04 | 5.00E−09 | 27 |
| H1M12386N | 405 | 65 | 5.40E+05 | 3.65E−04 | 6.76E−10 | 32 |
| H1M12388N | 408 | 59 | 8.15E+05 | 5.87E−04 | 7.20E−10 | 20 |
| H2aM12387N | 532 | 74 | 5.43E+05 | 5.40E−04 | 9.94E−10 | 21 |
| H3M12361N | 367 | 43 | 2.58E+05 | 8.05E−04 | 3.12E−09 | 14 |
| H2aM12452N | 348 | 35 | 8.13E+04 | 5.96E−04 | 7.33E−09 | 19 |
| H2aM12392N | 595 | 79 | 2.98E+05 | 9.22E−04 | 3.10E−09 | 13 |
| H2bM12454N | 485 | 61 | 3.84E+05 | 1.88E−03 | 4.89E−09 | 6 |
| H1M12362N | 308 | 51 | 1.13E+05 | 1.44E−03 | 1.27E−08 | 8 |
| H2bM13022N | 350 | 48 | 1.02E+05 | 1.32E−03 | 1.30E−08 | 9 |
| H1M12390N | 418 | 54 | 3.23E+05 | 3.36E−03 | 1.04E−08 | 3 |
| H2bM12385 | 536 | 48 | 3.22E+05 | 3.31E−03 | 1.03E−08 | 3 |
| H2aM12394N | 670 | 27 | 5.91E+04 | 5.53E−04 | 9.36E−09 | 21 |
| H3M12366N | 239 | 21 | IC | IC | IC | IC |
| H4H11990P2 | 707 | 94 | 1.93E+05 | 5.91E−03 | 3.06E−08 | 2 |
| H4H11997P2 | 819 | 106 | 1.86E+05 | 3.71E−03 | 1.99E−08 | 3.1 |
| H4H11998P2 | 633 | 11 | 2.64E+03 | 3.09E−02 | 1.17E−05 | 0.4 |
| H4H11996P2 | 628 | 61 | 1.65E+05 | 1.40E−02 | 8.49E−08 | 0.8 |
| H4H11995P2 | 643 | 8 | IC | IC | IC | IC |
| H4H11992P2 | 743 | 53 | 1.25E+05 | 2.67E−02 | 2.13E−07 | 0.4 |
| H4H11993P2 | 601 | 10 | IC | IC | IC | IC |

IC = inconclusive binding

TABLE 7
Binding kinetics parameters of human CD63 EC loop2-MMH
binding to anti-CD63 monoclonal antibodies at 37° C.

| | mAb | | | | | |
|---|---|---|---|---|---|---|
| | Capture | 100 nM Ag | | | | |
| | Level | Bound | kₐ | k_{d} | K_{D} | t½ |
| Antibody | (RU) | (RU) | (1/Ms) | (1/s) | (M) | (min) |
| H2aM12450N | 617 | 58 | 1.75E+05 | 2.01E−04 | 1.15E−09 | 57 |
| H2aM12395N | 535 | 31 | 1.65E+05 | 9.32E−03 | 5.64E−08 | 1 |
| H1M12451N | 597 | 44 | 8.89E+04 | 1.90E−03 | 2.14E−08 | 6 |
| H1M12386N | 455 | 65 | 6.50E+05 | 2.35E−03 | 3.61E−09 | 5 |
| H1M12388N | 462 | 60 | 6.51E+05 | 3.27E−03 | 5.02E−09 | 4 |
| H2aM12387N | 590 | 77 | 6.79E+05 | 3.54E−03 | 5.21E−09 | 3 |
| H3M12361N | 425 | 39 | 4.95E+05 | 2.89E−03 | 5.85E−09 | 4 |
| H2aM12452N | 421 | 34 | 3.85E+05 | 2.69E−03 | 6.98E−09 | 4 |
| H2aM12392N | 626 | 68 | 4.47E+05 | 3.24E−03 | 7.25E−09 | 4 |
| H2bM12454N | 586 | 56 | 5.59E+05 | 5.91E−03 | 1.06E−08 | 2 |
| H1M12362N | 369 | 32 | 3.69E+05 | 7.20E−03 | 1.95E−08 | 2 |
| H2bM13022N | 395 | 31 | 3.11E+05 | 6.71E−03 | 2.15E−08 | 2 |
| H1M12390N | 440 | 38 | 4.19E+05 | 1.49E−02 | 3.56E−08 | 1 |
| H2bM12385 | 642 | 44 | 2.71E+05 | 1.09E−02 | 4.00E−08 | 1 |
| H2aM12394N | 753 | 19 | 3.62E+04 | 2.77E−03 | 7.67E−08 | 4 |
| H3M12366N | 292 | 0 | NB | NB | NB | NB |
| H4H11990P2 | 840 | 81 | 1.60E+05 | 1.98E−02 | 1.24E−07 | 0.6 |
| H4H11997P2 | 899 | 74 | 1.32E+05 | 1.92E−02 | 1.45E−07 | 0.6 |
| H4H11998P2 | 639 | 3 | 5.28E+05 | 9.17E−02 | 1.74E−07 | 0.1 |
| H4H11996P2 | 704 | 46 | 1.08E+05 | 3.89E−02 | 3.61E−07 | 0.3 |
| H4H11995P2 | 674 | 3 | 7.94E+04 | 1.28E−01 | 1.62E−06 | 0.1 |
| H4H11992P2 | 889 | 30 | 6.60E+03 | 8.14E−02 | 1.23E−05 | 0.1 |
| H4H11993P2 | 723 | 3 | IC | IC | IC | IC |

IC = inconclusive binding
NB = no binding

TABLE 8
Binding kinetics parameters of human CD63 EC loop2-hFC
binding to anti-CD63 monoclonal antibodies at 25° C.

| | mAb | | | | | |
|---|---|---|---|---|---|---|
| | Capture | 100 nM Ag | | | | |
| | Level | Bound | kₐ | k_{d} | K_{D} | t½ |
| Antibody | (RU) | (RU) | (1/Ms) | (1/s) | (M) | (min) |
| H2aM12450N | 592 | 134 | 3.20E+05 | 4.66E−05 | 1.50E−10 | 248 |
| H2aM12395N | 472 | 117 | 6.77E+05 | 5.10E−04 | 7.53E−10 | 23 |
| H1M12451N | 525 | 107 | 1.19E+05 | 8.40E−05 | 7.04E−10 | 138 |
| H1M12386N | 405 | 160 | 6.54E+05 | 1.13E−04 | 1.73E−10 | 102 |
| H1M12388N | 408 | 131 | 7.28E+05 | 1.73E−04 | 2.38E−10 | 67 |
| H2aM12387N | 532 | 164 | 8.54E+05 | 1.10E−04 | 1.29E−10 | 105 |
| H3M12361N | 367 | 83 | 4.41E+05 | 1.32E−04 | 2.99E−10 | 87 |
| H2aM12452N | 348 | 91 | 1.22E+05 | 2.28E−04 | 1.87E−09 | 51 |
| H2aM12392N | 595 | 169 | 5.10E+05 | 1.99E−04 | 3.91E−10 | 58 |
| H2bM12454N | 485 | 142 | 6.88E+05 | 3.60E−04 | 5.23E−10 | 32 |
| H1M12362N | 308 | 125 | 4.77E+05 | 3.99E−04 | 8.40E−10 | 29 |
| H2bM13022N | 350 | 117 | 4.60E+05 | 2.80E−04 | 6.10E−10 | 41 |
| H1M12390N | 418 | 150 | 5.23E+05 | 9.91E−04 | 1.89E−09 | 12 |
| H2bM12385 | 536 | 121 | 5.92E+05 | 9.84E−04 | 1.66E−09 | 12 |
| H2aM12394N | 670 | 59 | 8.18E+04 | 1.44E−04 | 1.76E−09 | 80 |
| H3M12366N | 239 | 51 | IC | IC | IC | IC |
| H4H11990P2 | 707 | 275 | 4.30E+05 | 8.99E−05 | 2.09E−10 | 128 |
| H4H11997P2 | 819 | 291 | 4.17E+05 | 6.20E−05 | 1.49E−10 | 186 |
| H4H11998P2 | 633 | 136 | 1.75E+05 | 4.53E−04 | 2.58E−09 | 26 |
| H4H11996P2 | 628 | 257 | 4.61E+05 | 1.35E−04 | 2.92E−10 | 86 |
| H4H11995P2 | 643 | 198 | 5.52E+05 | 5.55E−03 | 1.01E−08 | 2 |
| H4H11992P2 | 743 | 304 | 6.41E+05 | 2.57E−04 | 4.01E−10 | 45 |
| H4H11993P2 | 601 | 194 | 3.64E+05 | 7.28E−04 | 2.00E−09 | 16 |

IC = inconclusive binding

TABLE 9
Binding kinetics parameters of human CD63 EC loop2-hFC
binding to anti-CD63 monoclonal antibodies at 37° C.

| | mAb | | | | | |
|---|---|---|---|---|---|---|
| | Capture | 100 nM Ag | | | | |
| | Level | Bound | kₐ | k_{d} | K_{D} | t½ |
| Antibody | (RU) | (RU) | (1/Ms) | (1/s) | (M) | (min) |
| H2aM12450N | 617 | 138 | 4.11E+05 | 4.89E−05 | 1.19E−10 | 236 |
| H2aM12395N | 535 | 123 | 5.29E+05 | 1.79E−03 | 3.38E−09 | 6 |
| H1M12451N | 597 | 118 | 6.51E+05 | 3.31E−04 | 5.09E−10 | 35 |
| H1M12386N | 455 | 170 | 9.21E+05 | 6.31E−04 | 6.85E−10 | 18 |
| H1M12388N | 462 | 158 | 1.03E+06 | 8.87E−04 | 8.63E−10 | 13 |
| H2aM12387N | 590 | 196 | 1.10E+06 | 6.57E−04 | 5.96E−10 | 18 |
| H3M12361N | 425 | 97 | 6.51E+05 | 3.70E−04 | 5.68E−10 | 31 |
| H2aM12452N | 421 | 104 | 7.79E+05 | 8.57E−04 | 1.10E−09 | 13 |
| H2aM12392N | 626 | 177 | 7.65E+05 | 4.45E−04 | 5.82E−10 | 26 |
| H2bM12454N | 586 | 160 | 8.68E+05 | 7.77E−04 | 8.95E−10 | 15 |
| H1M12362N | 369 | 118 | 6.85E+05 | 6.69E−04 | 9.77E−10 | 17 |
| H2bM13022N | 395 | 107 | 5.57E+05 | 5.62E−04 | 1.01E−09 | 21 |
| H1M12390N | 440 | 134 | 9.07E+05 | 4.95E−04 | 5.46E−10 | 23 |
| H2bM12385 | 642 | 133 | 8.27E+05 | 1.70E−03 | 2.05E−09 | 7 |
| H2aM12394N | 753 | 67 | 3.27E+05 | 5.04E−04 | 1.54E−09 | 23 |
| H3M12366N | 292 | 0 | NB | NB | NB | NB |
| H4H11990P2 | 840 | 338 | 4.82E+05 | 4.96E−05 | 1.04E−10 | 233 |
| H4H11997P2 | 899 | 342 | 4.25E+05 | 1.91E−05 | 4.50E−11 | 603 |
| H4H11998P2 | 639 | 111 | 3.18E+05 | 2.07E−03 | 6.50E−09 | 6 |
| H4H11996P2 | 704 | 294 | 5.38E+05 | 1.42E−04 | 2.64E−10 | 81 |
| H4H11995P2 | 674 | 116 | 1.29E+06 | 1.88E−02 | 1.45E−08 | 1 |
| H4H11992P2 | 889 | 358 | 6.47E+05 | 6.82E−04 | 1.05E−09 | 17 |
| H4H11993P2 | 723 | 156 | 5.77E+05 | 4.57E−03 | 7.92E−09 | 3 |

IC = inconclusive binding

### Octet Cross-Competition Between Different Anti-CD63 Monoclonal Antibodies

Binding competition between a panel of anti-CD63 monoclonal antibodies was determined using a real time, label-free bio-layer interferometry assay on the Octet HTX biosensor platform (Pall ForteBijo Corp.). The entire experiment was performed at 25° C. in 10 mM HEPES, 150 mM NaCl, 3 mM EDTA, and 0.05% v/v Surfactant Tween-20, 1 mg/mL BSA, pH7.4 (HBS-EBT) buffer with the plate shaking at the speed of 1000 rpm. To assess whether 2 antibodies are able to compete with one another for binding to their respective epitopes on the recombinant human CD63 EC loop 2 expressed with a C-terminal myc-myc-hexahistidine tag (hCD63 EC Loop 2-MMH; SEQ ID: xx), around ˜0.61 nm of hCD63 EC Loop 2-MMH was first captured onto anti-Penta-His antibody coated Octet biosensor tips (Fortebio Inc, #18-5122) by submerging the biosensor tips for 3 minutes in wells containing 20 μg/mL solution of hCD63 EC Loop 2-MMH. The antigen captured biosensor tips were then saturated with first anti-CD63 monoclonal antibody (subsequently referred to as mAb-1) by dipping into wells containing 50 μg/mL solution of mAb-1 for 210 seconds. The biosensor tips were then subsequently dipped into wells containing 50 μg/mL solution of a second anti-CD63 monoclonal antibody (subsequently referred to as mAb-2) for 120 seconds. The biosensor tips were washed in HBS-EBT buffer in between every step of the experiment. The real-time binding response was monitored during the entire course of the experiment and the binding response at the end of every step was recorded. The response of mAb-2 binding to hCD63 EC Loop 2-MMH pre-complexed with mAb-1 was compared and competitive/non-competitive behavior of different anti-CD63 monoclonal antibodies was determined as shown in Table 10.

TABLE 10
Cross-competition between anti-CD63 monoclonal antibodies

| | mAb-2 Competing |
|---|---|
| mAb-1 | with mAb-1 |
| H1M12451N | H2aM12394N |
| | H2aM12395N |
| | H2aM12450N |
| | H2aM12452N |
| | H3M12366N |
| | H2bM12454N |
| | H2bM13022N |
| | H4H11992P2 |
| | H4H11993P2 |
| | H4H11995P2 |
| | H4H11996P2 |
| | H4H11997P2 |
| | H4H11998P2 |
| | H4H11990P2 |
| | H1M12386N |
| | H1M12390N |
| | H2aM12387N |
| | H2aM12392N |
| | H3M12361N |
| H2aM12394N | H1M12451N |
| | H2aM12395N |
| | H2aM12450N |
| | H2aM12452N |
| | H3M12366N |
| | H2bM12454N |
| | H2bM13022N |
| | H4H11992P2 |
| | H4H11993P2 |
| | H4H11995P2 |
| | H4H11996P2 |
| | H4H11997P2 |
| | H4H11998P2 |
| | H4H11990P2 |
| | H1M12386N |
| | H1M12390N |
| | H2aM12387N |
| | H2aM12392N |
| | H3M12361N |
| H2aM12395N | H1M12451N |
| | H2aM12394N |
| | H2aM12450N |
| | H2aM12452N |
| | H3M12366N |
| | H2bM12454N |
| | H2bM13022N |
| | H4H11992P2 |
| | H4H11993P2 |
| | H4H11995P2 |
| | H4H11996P2 |
| | H4H11997P2 |
| | H4H11998P2 |
| | H4H11990P2 |
| | H1M12386N |
| | H1M12390N |
| | H2aM12387N |
| | H2aM12392N |
| | H3M12361N |
| H2aM12450N | H1M12451N |
| | H2aM12394N |
| | H2aM12395N |
| | H2aM12452N |
| | H3M12366N |
| | H2bM12454N |
| | H2bM13022N |
| | H4H11992P2 |
| | H4H11993P2 |
| | H4H11995P2 |
| | H4H11996P2 |
| | H4H11997P2 |
| | H4H11998P2 |
| | H4H11990P2 |
| | H1M12386N |
| | H1M12390N |
| | H2aM12387N |
| | H2aM12392N |
| | H3M12361N |
| H2aM12452N | H1M12451N |
| | H2aM12394N |
| | H2aM12395N |
| | H2aM12450N |
| | H3M12366N |
| | H2bM12454N |
| | H2bM13022N |
| | H4H11992P2 |
| | H4H11993P2 |
| | H4H11995P2 |
| | H4H11996P2 |
| | H4H11997P2 |
| | H4H11998P2 |
| | H4H11990P2 |
| | H1M12386N |
| | H1M12390N |
| | H2aM12387N |
| | H2aM12392N |
| | H3M12361N |
| H3M12366N | H1M12451N |
| | H2aM12394N |
| | H2aM12395N |
| | H2aM12450N |
| | H2aM12452N |
| | H2bM12454N |
| | H2bM13022N |
| | H4H11992P2 |
| | H4H11993P2 |
| | H4H11995P2 |
| | H4H11996P2 |
| | H4H11997P2 |
| | H4H11998P2 |
| | H4H11990P2 |
| | H1M12386N |
| | H1M12390N |
| | H2aM12387N |
| | H2aM12392N |
| | H3M12361N |
| H2bM12454N | H1M12451N |
| | H2aM12394N |
| | H2aM12395N |
| | H2aM12450N |
| | H2aM12452N |
| | H3M12366N |
| | H2bM13022N |
| | H4H11992P2 |
| | H4H11993P2 |
| | H4H11995P2 |
| | H4H11996P2 |
| | H4H11997P2 |
| | H4H11998P2 |
| | H4H11990P2 |
| | H1M12386N |
| | H1M12390N |
| | H2aM12387N |
| | H2aM12392N |
| H2bM13022N | H1M12451N |
| | H2aM12394N |
| | H2aM12395N |
| | H2aM12450N |
| | H2aM12452N |
| | H3M12366N |
| | H2bM12454N |
| | H4H11992P2 |
| | H4H11993P2 |
| | H4H11995P2 |
| | H4H11996P2 |
| | H4H11997P2 |
| | H4H11998P2 |
| | H4H11990P2 |
| | H1M12386N |
| | H1M12390N |
| | H2aM12387N |
| | H2aM12392N |
| H4H11992P2 | H1M12451N |
| | H2aM12394N |
| | H2aM12395N |
| | H2aM12450N |
| | H2aM12452N |
| | H3M12366N |
| | H2bM12454N |
| | H2bM13022N |
| | H4H11993P2 |
| | H4H11995P2 |
| | H4H11996P2 |
| | H4H11997P2 |
| | H4H11998P2 |
| | H4H11990P2 |
| | H1M12386N |
| | H1M12390N |
| | H2aM12387N |
| | H2aM12392N |
| H4H11993P2 | H1M12451N |
| | H2aM12394N |
| | H2aM12395N |
| | H2aM12450N |
| | H2aM12452N |
| | H3M12366N |
| | H2bM12454N |
| | H2bM13022N |
| | H4H11992P2 |
| | H4H11995P2 |
| | H4H11996P2 |
| | H4H11997P2 |
| | H4H11998P2 |
| | H4H11990P2 |
| | H1M12386N |
| | H1M12390N |
| | H2aM12387N |
| | H2aM12392N |
| H4H11995P2 | H1M12451N |
| | H2aM12394N |
| | H2aM12395N |
| | H2aM12450N |
| | H2aM12452N |
| | H3M12366N |
| | H2bM12454N |
| | H2bM13022N |
| | H4H11992P2 |
| | H4H11993P2 |
| | H4H11996P2 |
| | H4H11997P2 |
| | H4H11998P2 |
| | H4H11990P2 |
| | H1M12386N |
| | H1M12390N |
| | H2aM12387N |
| | H2aM12392N |
| H4H11996P2 | H1M12451N |
| | H2aM12394N |
| | H2aM12395N |
| | H2aM12450N |
| | H2aM12452N |
| | H3M12366N |
| | H2bM12454N |
| | H2bM13022N |
| | H4H11992P2 |
| | H4H11993P2 |
| | H4H11995P2 |
| | H4H11997P2 |
| | H4H11998P2 |
| | H4H11990P2 |
| | H1M12386N |
| | H1M12390N |
| | H2aM12387N |
| | H2aM12392N |
| H4H11997P2 | H1M12451N |
| | H2aM12394N |
| | H2aM12395N |
| | H2aM12450N |
| | H2aM12452N |
| | H3M12366N |
| | H2bM12454N |
| | H2bM13022N |
| | H4H11992P2 |
| | H4H11993P2 |
| | H4H11995P2 |
| | H4H11996P2 |
| | H4H11998P2 |
| | H4H11990P2 |
| | H1M12386N |
| | H1M12390N |
| | H2aM12387N |
| | H2aM12392N |
| H4H11998P2 | H1M12451N |
| | H2aM12394N |
| | H2aM12395N |
| | H2aM12450N |
| | H2aM12452N |
| | H3M12366N |
| | H2bM12454N |
| | H2bM13022N |
| | H4H11992P2 |
| | H4H11993P2 |
| | H4H11995P2 |
| | H4H11996P2 |
| | H4H11997P2 |
| | H4H11990P2 |
| | H1M12386N |
| | H1M12390N |
| | H2aM12387N |
| | H2aM12392N |
| H4H11990P2 | H1M12451N |
| | H2aM12394N |
| | H2aM12395N |
| | H2aM12450N |
| | H2aM12452N |
| | H3M12366N |
| | H2bM12454N |
| | H2bM13022N |
| | H4H11992P2 |
| | H4H11993P2 |
| | H4H11995P2 |
| | H4H11996P2 |
| | H4H11997P2 |
| | H4H11998P2 |
| | H1M12386N |
| | H1M12390N |
| | H2aM12387N |
| | H2aM12392N |
| H1M12386N | H1M12451N |
| | H2aM12394N |
| | H2aM12395N |
| | H2aM12450N |
| | H2aM12452N |
| | H3M12366N |
| | H2bM12454N |
| | H2bM13022N |
| | H4H11992P2 |
| | H4H11993P2 |
| | H4H11995P2 |
| | H4H11996P2 |
| | H4H11997P2 |
| | H4H11998P2 |
| | H4H11990P2 |
| | H1M12390N |
| | H2aM12387N |
| | H2aM12392N |
| H1M12390N | H1M12451N |
| | H2aM12394N |
| | H2aM12395N |
| | H2aM12450N |
| | H2aM12452N |
| | H3M12366N |
| | H2bM12454N |
| | H2bM13022N |
| | H4H11992P2 |
| | H4H11993P2 |
| | H4H11995P2 |
| | H4H11996P2 |
| | H4H11997P2 |
| | H4H11998P2 |
| | H4H11990P2 |
| | H1M12386N |
| | H2aM12387N |
| | H2aM12392N |
| H2aM12387N | H1M12451N |
| | H2aM12394N |
| | H2aM12395N |
| | H2aM12450N |
| | H2aM12452N |
| | H3M12366N |
| | H2bM12454N |
| | H2bM13022N |
| | H4H11992P2 |
| | H4H11993P2 |
| | H4H11995P2 |
| | H4H11996P2 |
| | H4H11997P2 |
| | H4H11998P2 |
| | H4H11990P2 |
| | H1M12386N |
| | H1M12390N |
| | H2aM12392N |
| H2aM12392N | H1M12451N |
| | H2aM12394N |
| | H2aM12395N |
| | H2aM12450N |
| | H2aM12452N |
| | H3M12366N |
| | H2bM12454N |
| | H2bM13022N |
| | H4H11992P2 |
| | H4H11993P2 |
| | H4H11995P2 |
| | H4H11996P2 |
| | H4H11997P2 |
| | H4H11998P2 |
| | H4H11990P2 |
| | H1M12386N |
| | H1M12390N |
| | H2aM12387N |
| H3M12361N | H1M12451N |
| | H2aM12394N |
| | H2aM12395N |
| | H2aM12450N |
| | H2aM12452N |
| | H3M12366N |

### Generation of Bispecific Antibodies to Determine Internalization of Bispecific Complex Having an Anti-CD63 Binding Arm

To assess the ability of anti-CD63 antibodies generated as described in this example to internalize as part of a bispecific antigen-binding molecule, the antibodies were reconstructed into bispecific formats where one binding arm was the anti-CD63 antibody VH/VL pair (see Table 1 parental antibodies) and the other was an irrelevant binding arm. Standard methods of making bispecific antibodies were used, and exemplary methods are described in, e.g., US Application Publication No. 2010/0331527, and U.S. Pat. No. 5,731,168, each of which is incorporated herein by reference. The bispecific antibodies were tested for their ability to internalize using human CD63 expressing cells. For the assay, HEK293 cells, which endogenously express human CD63, were plated at a density of 10,000 cells/well in DMEM containing 10% FBS and penicillin-streptomycin/L-glutamine (Gibco, Cat #10378016) in clear bottom black Poly-D-Lysine coated 96-well plates (Greiner, Cat #655946). Two days later, the media was replaced with fresh media containing anti-CD63 bispecific antibodies and a negative control antibody in a 2-fold dilution series beginning at 10 μg/mL to 0.157 μg/mL, along with a media only control. Cells were then incubated at 37° C. for 3 hours to allow for antibody internalization. Following the incubation, cells were washed with PBS, fixed in 4% paraformaldehyde (Thermo Scientific, Cat #28908) for 20 minutes at room temperature, and subsequently permeabilized with 0.2% Triton X-100 (Spectrum Chemical, Cat #TR135) in 5% normal goat serum (NGS) (Gibco, Cat #PCN5000) for 20 minutes at room temperature. Cells were then incubated with either 2 μg/mL of donkey anti-mouse IgG Alexa Fluor-647 Fab (Manufacture, Cat #115-606-006) or 2 μg/mL of goat anti-human IgG Alexa Fluor-647 Fab (Jackson ImmunoResearch, Cat #115-606-006) in 5% NGS for 1 hour at room temperature. The secondary antibody solution was removed, then cells were washed with PBS, and subsequently fresh PBS containing 2 drops/mL of NucBlue (Invitrogen, CAT #R37605) was added to stain live cell nuclei. Antibody internalization and nuclei were imaged at 40x on the ImageXpress High-Content Imaging System (Molecular Devices) and antibody internalization was quantified using the MetaXpress Software Transfluor Application Module (Molecular Devices). Antibody internalization is reported as pit integrated intensity per cell ±standard deviation (SD).

As shown in Table 11, all of the bispecific antibodies incorporating a single arm binding to human CD63 (derived from either H1M12451, H2M12450, or H2M12395) and an irrelevant non-binding arm demonstrated efficient internalization into HEK293 cells. The bispecific antibody incorporating one binding arm of mAb12450 demonstrated a higher amount of internalization than the other bispecific antibodies tested.

TABLE 11
Internalization of anti-CD63 bispecific antibodies by HEK293 cells

| Concentration | Internalization of antibody (pit integrated intensity) ± SD | | | |
|---|---|---|---|---|
| of antibody | H2M12395N | H2M12450N | H1M12451N | Negative |
| (μg/mL) | bispecific | bispecific | bispecific | control Ab |
| 10 | 1.05E+06 ± | 4.34E+06 ± | 8.26E+05 ± | 4.58E+03 ± |
| | 4.56E+05 | 8.77E+05 | 2.67E+05 | 6.50E+03 |
| ~5 | 1.07E+06 ± | 4.31E+06 ± | 5.45E+05 ± | 1.23E+03 ± |
| | 4.06E+05 | 5.48E+05 | 5.20E+04 | 8.85E+02 |
| 2.5 | 2.73E+05 ± | 3.92E+06 ± | 3.27E+05 ± | 7.70E+02 ± |
| | 6.01E+04 | 5.80E+05 | 1.06E+05 | 6.09E+02 |
| 1.25 | 1.89E+05 ± | 2.72E+06 ± | 1.20E+05 ± | 1.43E+03 ± |
| | 6.61E+04 | 2.63E+05 | 3.91E+04 | 1.19E+03 |
| 0.625 | 2.03E+05 ± | 1.75E+06 ± | 7.87E+04 ± | 3.37E+03 ± |
| | 9.37E+04 | 1.39E+05 | 1.07E+04 | 5.22E+03 |
| 0.3125 | 3.95E+04 ± | 8.57E+05 ± | 3.77E+04 ± | 1.99E+03 ± |
| | 8.23E+03 | 1.60E+05 | 1.22E+04 | 2.19E+03 |
| 0.15625 | 2.81E+04 ± | 2.42E+05 ± | 7.23E+03 ± | 1.87E+03 ± |
| | 1.30E+04 | 2.54E+04 | 6.09E+03 | 1.16E+03 |
| 0 | 3.72E+03 ± | 8.21E+03 ± | 1.66E+04 ± | 1.10E+03 ± |
| | 1.66E+03 | 3.47E+03 | 1.80E+04 | 1.56E+03 |

### Example 2: Construction of Anti-hCD63 ScFv::GAA Polynucleotide and Gene Therapy Vector

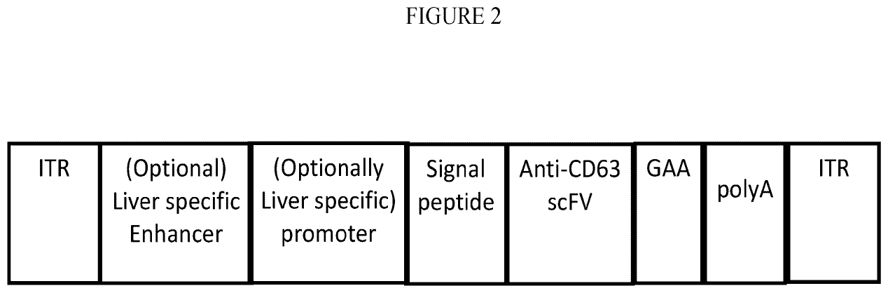

AAV2/8 viruses encoding for the expression of human GAA (hGAA; SEQ ID NO: 369) or an anti-human CD63 single chain variable fragment (ScFv) fused on its C-terminus to human GAA (anti-hCD63 ScFv-hGAA) are generated using a standard triple transfection protocol (Gray et al. 2011; see also “Production of recombinant adeno-associated viral vectors and use in vitro and in vivo administration”, Current Protocols in Neuroscience, John Wiley & Sons, New York (1999), pp. 4.17.1-4.17.25, Vol 1). For the production, 1×10⁷ HEK293 cells are plated onto 15 cm plates. The following day the cells are transfected with (A) either 8 g of a control pAAV vector comprising a liver specific serpina 1 enhancer (SEQ ID NO: 367) and encoding TTR promoter (SEQ ID NO: 368) driven human GAA or test pAAV comprising a liver specific serpina 1 enhancer (SEQ ID NO: 367) and encoding a TTR promoter (SEQ ID NO: 368) driven hCD63 ScFv-hGAA (see, e.g., FIG. 2) and (B) pAAV RC2/8-derived vector (Gao, 2002) and 16 g of pHelper (Agilent, Cat #240074) using PEIpro (Polyplus transfection, New York, NY catalog #115-100)-mediated transfection at ratio of 1:1 (1 ul PEIpro: 1 μg DNA). Seventy-two hours after transfection, the cells are collected and lysed in a buffer comprised of 20 mM Tris-HCl, 1 mM MgCl2, 2.5 mM KCl, 100 mM NaCl using a standard freeze-thaw method. Next, benzonase (Sigma, Cat #E1014-25KU) is added to the samples at a final concentration of 0.5 U/L, and then incubated at 37° C. for 60 minutes. Viruses are purified using iodixanol gradient ultracentrifugation as described in (Zolotukhin et al., 1999, Gene Ther 1999; 6:973-985) and subsequently titrated by qPCR.

AAV samples are treated with DNaseI (Thermofisher Scientific, Cat #EN0525) at 37° C. for one hour and lysed using DNA extract All Reagents (Thermofisher Scientific Cat #4403319). Encapsidated viral genomes are quantified using an QuantStudio 3 Real-Time PCR System (Thermofisher Scientific) using primers directed to the AAV2 ITRs. The sequences of the AAV2 ITRs primers are 5′-GGAACCCCTAGTGATGGAGTT-3′ (fwd ITR; SEQ ID NO: 370) and 5′-CGGCCTCAGTGAGCGA-3′ (rev ITR; SEQ ID NO: 371) (Aurnhammer et al., 2012), derived the left internal inverted repeat (ITR) sequence from of the AAV (SEQ ID NO: 365) and the right internal inverted repeat (ITR) sequence from of the AAV (SEQ ID NO: 366), respectively. The sequence of the AAV2 ITRs probe is 5′-6-FAM-CACTCCCTCTCTGCGCGCTCG-TAMRA-3′ (SEQ ID NO: 372) (Aurnhammer C., Haase M., Muether N., et al., 2012, Hum. Gene Ther. Methods 23, 18-28). After a 95° C. activation step for 10 min, a two-step PCR cycle is performed at 95° C. for 15 seconds and 60° C. for 30 seconds for 40 cycles. The TaqMan Universal PCR Master Mix (Thermofisher Scientific, Cat #4304437) is used in the qPCR. DNA plasmid (Agilent, Cat #240074) is used as standard to determine absolute titers.

Anti-human CD63 antibodies and their fusions use the anti-CD63 variable domains set forth in Table 1. ScFv versions of the antibodies are cloned with variable domains in heavy-light order with a glycine-serine linker in between (5′-VH-Gly-Ser-VL-3′).

AAV encoding an anti-hCD63 scFv::GAA multidomain therapeutic having an amino acid sequence set forth as SEQ ID NO: 364 (FIG. 2) were generated and tested for efficacy in providing enzyme replacement treatment

### Example 3: Glycogen Content in Murine Pompe Model Post-AAV

To determine the effect of AAV delivered anti-hCD63 ScFv-GAA fusion (SEQ ID NO: 364) versus AAV delivered GAA, in a relevant glycogen storage in vivo model, both therapies are delivered to a Pompe disease mouse model where mice are homozygous for the deletion of the mouse GAA gene and are homozygous for the expression of human CD63 in place of mouse CD63 with a strain background of 75% C57BL/6; 25% 129SvJ. These mice are herein referred to as CD63 HumIn GAA KO mice or alternatively as CD63hu/hu; GAA^{-/-} mice.

For the experiment, 2-month-old CD63 HumIn GAA KO are administered via tail vein injection with either AAV2/8 virus containing a genome with either the TTR liver specific promoter driving human GAA (AAV-hGAA; described in Example 2) or the TTR liver specific promoter driving anti-human CD63 ScFv fused at its C-terminus with human GAA (AAV-anti-hCD63 ScFv-hGAA; described in Example 2). Both AAV2/8 viruses are delivered at either one of two doses, 1e10 vg/mouse or 1e11 vg/mouse. As controls, untreated CD63 HumIn GAA KO mice and untreated CD63 HumIn with the mouse GAA gene intact are included in the assay. Mice are housed for 3 months after treatment and bled incrementally (monthly) during this period for serum measurements of GAA levels and anti-GAA antibodies. After 3 months, all mice are sacrificed and individual tissues are harvested for glycogen measurements, PAS-H staining, quantification of central nuclei, measurement of lysosomal proliferation, and measurement of LC3b expression. Experimental dosing and treatment protocol for groups of mice are shown in Table 12.

TABLE 12
Experimental dosing and treatment protocol for groups of mice

| | | Number | | |
|---|---|---|---|---|
| Group | Mice | of Mice | Treatment | Dosage |
| 1 | CD63 Humin GAA KO | 4 | None | N/A |
| 2 | CD63 Humin GAA KO | 4 | AAV-hGAA | 1e10 |
| | | | | vg/mouse |
| 3 | CD63 Humin GAA KO | 4 | AAV-hGAA | 1e11 |
| | | | | vg/mouse |
| 4 | CD63 Humin GAA KO | 5 | AAV-anti- | 1e10 |
| | | | hCD63 | vg/mouse |
| | | | ScFv-hGAA | |
| 5 | CD63 Humin GAA KO | 4 | AAV-anti- | 1e11 |
| | | | hCD63 | vg/mouse |
| | | | ScFv-hGAA | |
| 6 | CD63 Humin GAA WT | 2 | None | N/A |

### Example 4: Immunological Response to GAA

To measure anti-human GAA antibody serum levels, serum from all the treatment groups is separated from the blood collected during the terminal bleed using serum separator tubes (BD Biosciences, Cat #365967) as per the manufacturer's specifications. Separately, 96-well high protein binding plates (ThermoFisher, Cat #15041) are coated with 20 μg of hGAA (R&D Systems, Cat #8329-GH-025) diluted in PBS overnight. Plates are washed with PBS+0.05% Tween (PBS-T) 3 times. Plates are blocked with 0.5% BSA in PBS-T, and serial dilutions of mouse serum ranging from 1:300 to 1:5.1e7 are added to the plate overnight. Total anti-mouse IgG (subclasses 1+2a+2b+3) is measured using a HRP conjugated goat anti-mouse IgG antibody (Jackson Immuno Research, Cat #115-035-164) and the BD Opt EIA substrate kit. The colormetric reactions are stopped using 1 N phosphoric acid. Absorbance is then read at 450 nm on a Spectramax i3 plate reader (Molecular Devices). Dilution curves are fit to sigmoidal curves, and titers are calculated from the curves.

In similarly performed experiments, higher levels of GAA or anti-hCD63scFv::GAA (SEQ ID NO: 364) after AAV administration was correlated with lower anti-GAA titers (data not shown). The serum of GAA null mice treated with high or low titers of AAV-anti-hCD63scFv::GAA (SEQ ID NO: 364) or AAV-GAA were assessed for anti-GAA antibodies over the course of the three months post-injection. A negative correlation between antibody titer and serum exposure to GAA, and an inverse correlation between doses of construct and titer of anti-GAA antibodies was seen (data not shown).

### Example 5: Serum GAA

To measure human GAA serum levels over the course of the experiment, samples are collected at monthly time points via tail bleed. Serum is separated from the blood using serum separator tubes (BD Biosciences, Cat #365967) as per the manufacturer's specifications. 1p L of isolated serum is then loaded onto a 4-20% Novex wedgewell pre-cast gel, run at 220V for 45 minutes and transferred to nitrocellulose membrane at 200 mA for 1 hour using standard procedures. The nitrocellulose membrane is then probed with an anti-GAA primary antibody (Abcam, #ab137068) used at a dilution of 1:2000 and an anti-GAPDH antibody (Abcam, #AB9484) used at a dilution of 1:1000 in 12 mL and incubated overnight at 4° C. After primary antibody incubation, the membrane is washed three times with 1× TBST for 5 minutes per wash. Anti-rabbit IgG (LiCor, 926-32211) and anti-mouse IgG (LiCor, 925-68070) (LiCor, Lincoln, NE) secondary antibodies at a dilution of 1:15000 in 12 mL are then added to the membrane and incubated for 1 hour at room temperature. After secondary antibody incubation, the membrane is washed two times with 1× TBST for 5 minutes per wash and one time with 1× TBS for 5 minutes. The membrane is then imaged and quantified using a LiCor Odyssey instrument (LI-COR Biotechnology).

In experiments similar to those described above, CD63 HumIn GAA KO mice treated with the high dose (10¹¹ vg/mouse) of AAV-anti-hCD63 ScFv-hGAA (SEQ ID NO: 364) or AAV-hGAA demonstrated sustained levels of GAA in the serum over the course of the experiment (data not shown).

Real-time PCR quantifications of expression in liver, heart, and quadriceps lysates 3 months after injection was performed. Liver expression was detected for all injections of AAV construct, and a comparison of serum GAA level to RNA expression level of GAA was also made. The data (not shown) suggested that the AAV encoding the fusion protein set forth as SEQ ID NO: 364 (and expression is driven by a liver-specific promoter) attains an improved secretion profile for GAA.

### Example 6: Tissue Measurement of Glycogen and Histological Characterization of Muscle Tissue

Tissue measurements of glycogen: To measure the glycogen content in individual tissues, heart, quadriceps, gastrocnemius, diaphragm, soleus, and EDL tissue are dissected from mice from all groups immediately after CO₂ asphyxiation, and then snap frozen in liquid nitrogen, and stored at −80° C. −50 mg of each tissue is lysed on a benchtop homogenizer with stainless steel beads in distilled water at a ratio of 1 mg to 25 μL water for glycogen measurements. Glycogen analysis lysates are heated at 105° for 15 minutes and centrifuged at 21000×g to clear debris. Glycogen measurements are performed using a Glycogen Assay Kit (Sigma-Aldrich, #MAK016) according to manufacturer's instructions for fluorometric assays. The fluorescence of each sample is measured at 535 nm excitation and 587 nm emission on a fluorescence plate reader (Molecular Devices, Spectramax i3). The calculated amount of glycogen is calculated using the following formula provided by the manufacturer.

Quadricep harvest for histopathology and quantification: Quadricep tissue samples from mice from each group besides the low dose (1e10 vg/mouse) treatment group are either snap frozen immediately after dissection in liquid nitrogen and stored at −80° C. for quantification of LC3b expression or are placed onto blocks containing O.C.T medium (Tissue-Tek, #4583).

Tissues samples in O.C.T medium are sent to Histoserv, Inc (Germantown, MD) for sectioning and periodic acid Schiff (PAS) staining to detect polysaccharides. Additional sections are prepared and returned for staining of central nuclei and lysosomal proliferation.

PAS staining: PAS stain sections are imaged using a Leica slide scanner at 20× magnification.

Quantification of central nuclei and lysosomal proliferation: Unstained sections from Histoserv are removed from the freezer and then fixed with 4% paraformaldehyde in PBS for 15 minutes in a staining chamber. The fixed slides are then washed twice for 5 minutes in PBS and subsequently incubated with blocking buffer (eBiosciences, 00-4953-54) for 1 hour at room temperature. Slides are then either stained with either a rat anti-Lamp-1 antibody (Abcam, #AB25245) at a dilution of 1:50 in blocking buffer, a rabbit anti-Laminin antibody (Sigma, #L9393) at a dilution of 1:1000 in blocking buffer, or blocking buffer with no added antibody while in a humidified staining chamber and then transferred to 4° C. for overnight incubation. The following day, slides are then washed twice for 5 minutes in PBS and subsequently stained with either goat anti-rabbit IgG (H+L) superclonal secondary antibody conjugated with Alexa Fluor 647 (Life Tech Thermo, #A27040) or goat anti-rat IgG (H+L) cross-adsorbed secondary antibody conjugated with Alexa Fluor 555 (Life Tech Thermo, #A21434) in a staining chamber then allowed to incubate for 1 hour at room temperature. Stained slides are then washed twice for 5 minutes in PBS before they being mounted with Fluoromount-G with DAPI (Life Tech Thermo, #00-4959-52) and imaged on a Zeiss LSM710 instrument (Carl Zeiss Microscopy GmbH). The number of centralized nuclei is quantified using Halo software (Indica Labs, NM)

Quantification of LC3b expression: For quantification of LC3b expression, snap frozen samples are thawed, homogenized and then lysed in RIPA buffer at a 1 mg tissue to 25 μL RIPA buffer ratio (150 mM NaCl, 1.0% IGEPAL® CA-630, 0.5% sodium deoxycholate, 0.1% SDS, 50 mM Tris, pH 8.0, Sigma Aldrich, R0278) by bead impaction for 45 seconds (MP Biomedical). Lysates are cleared of insoluble material by centrifugation at 21,000×g and then 300 μg of lysate in RIPA buffer is loaded on a 4-20% Novex wedgewell pre-cast gel, transferred to a nitrocellulose membrane and analyzed by western blot using a similar protocol as previously described for the analysis of serum GAA levels, substituting the use of primary antibody that recognizes mouse LC3b-I and LC3b-II (Sigma, #L7543) in place of the primary antibody against GAA. The membrane was then imaged and quantified using a LiCor Odyssey instrument (LI-COR Biotechnology). LC3b-I and LC3b-II levels expressed as (mean+/−standard deviation) in arbitrary units.

1.-25. (canceled)

26. An anti-CD63 antibody or antigen-binding fragment thereof, wherein the antibody or antigen-binding fragment thereof comprises three heavy chain complementarity determining regions (HCDRs) within a heavy chain variable region (HCVR) amino acid sequence and three light chain complementarity determining regions (LCDRs) within a light chain variable region (LCVR) amino acid sequence of the HCVR/LCVR pair set forth in SEQ ID NOs: 34/42.

27. The anti-CD63 antibody or antigen-binding fragment thereof of claim 26, wherein the antibody or antigen-binding fragment thereof comprises the set of six CDRs set forth in SEQ ID NOs: 36-38-40-44-46-48.

28. The anti-CD63 antibody or antigen-binding fragment thereof of claim 26, wherein the antibody or antigen-binding fragment thereof comprises the HCVR/LCVR amino acid sequence pair set forth in SEQ ID NOs: 34/42.

29. A bispecific antigen-binding molecule comprising:
(i) a first antigen-binding domain comprising the anti-CD63 antibody or antigen-binding fragment thereof of claim 26, and
(ii) a second antigen-binding domain that binds a target antigen.

30. The bispecific antigen-binding molecule of claim 29, wherein the target antigen is a tumor associated antigen.

31. The bispecific antigen-binding molecule of claim 29, wherein each of the first antigen-binding domain and the second antigen-binding domain is fully human.

32. The bispecific antigen-binding molecule of claim 29, wherein the bispecific antigen-binding molecule binds both human CD63 and a human target antigen expressed on a cell and induces CD63 internalization and/or degradation of the human target antigen in that cell.

33. The bispecific antigen-binding molecule of claim 29, wherein the bispecific antigen-binding molecule is fully human.

34. A multidomain therapeutic protein comprising the anti-CD63 antibody or antigen-binding fragment thereof of claim 26 and an enzyme domain.

35. The multidomain therapeutic protein of claim 34, wherein the enzyme domain comprises GAA or a biologically active portion thereof.

36. A polynucleotide comprising a sequence encoding the anti-CD63 antibody or antigen-binding fragment thereof of claim 26.

37. A polynucleotide comprising a sequence encoding the multidomain therapeutic protein of claim 34.

38. The polynucleotide of claim 37, wherein the polynucleotide is within an AAV vector.

39. The polynucleotide of claim 38, wherein the polynucleotide further comprises the sequences set forth in SEQ ID NO: 365 and SEQ ID NO: 366.

40. The polynucleotide of claim 39, wherein the polynucleotide further comprises a liver-specific enhancer and/or liver-specific promoter.

41. The polynucleotide of claim 40, wherein the liver-specific enhancer comprises the sequence set forth in SEQ ID NO: 367 and/or the liver-specific promoter comprises the sequence set forth in SEQ ID NO: 368.

42. A pharmaceutical composition comprising the anti-CD63 antibody or antigen-binding fragment thereof of claim 26 and a pharmaceutically acceptable carrier.
